(19) United States
(12) Patent Application Publication
(10) Pub. No.: US 2026/0335278 A1
(43) Pub. Date: Oct. 29, 2026

(54) PHYTOCHEMICAL FORMULATIONS FOR TREATING STRESS AND ANXIETY

(71) Applicant: GBS GLOBAL BIOPHARMA, INC., Ottwa (CA)

(72) Inventors: Andrea SMALL-HOWARD, Norwalk, CA (US); Helen Cathryn TURNER, Honolulu, HI (US)

(21) Appl. No.: 19/109,064

(22) PCT Filed: Sep. 5, 2023
(86) PCT No.: PCT/US2023/032021

§ 371 Date: Mar. 5, 2025

Related U.S. Application Data
(60) Provisional application No. 63/374,584, filed on Sep. 5, 2022.

(51) Int. Cl. A61K 31/366; A61K 31/00; A61K 31/01; A61K 31/015; A61K 31/12; A61K 31/352; A61K 31/4412; A61K 31/4453; A61P 25/22

(52) U.S. Cl. CPC A61K 31/366; A61K 31/01; A61K 31/015; A61K 31/12; A61K 31/352; A61K 31/4412; A61K 31/4453; A61K 31/658; A61P 25/22

(57) ABSTRACT

The embodiments disclose a method of treating stress, anxiety, and anxiety disorders in a human, comprising administering to a human in need of such treatment an effective amount of a pharmaceutical treatment derived from selective Piper methysticum extracts or individual phytochemicals used singly, in pairs, or in triplets, as described. Alternate formulations utilizing analogous compounds from other Piper plant species or other plants used in traditional medicine also showed promise for the treatment of stress, anxiety, and anxiety related disorders.

### CROSS-REFERENCED TO RELATED APPLICATIONS

This Patent Application is a U.S. National Phase Application, filed under 35 U.S.C. § 371 (c), of International Patent Application No. PCT/US2023/032021, filed Sep. 5, 2023, which claims benefit of U.S. Provisional Application Ser. No. 63/374,584 filed Sep. 5, 2022, entitled “FORMULATIONS FOR TREATING ANXIETY”, the entirety of each of which are hereby incorporated herein by reference.

### BACKGROUND

Stress, anxiety, and anxiety disorders are a group of mental health conditions characterized by excessive and often irrational feelings of fear, worry, and apprehension. They can significantly impact a person's daily life, making even routine tasks challenging to undertake. Common anxiety disorders include generalized anxiety disorder (GAD), panic disorder, social anxiety disorder, and specific phobias. These conditions can manifest with a wide range of symptoms, including racing thoughts, restlessness, muscle tension, rapid heartbeat, and even physical symptoms like sweating and trembling. Discovery of effective treatments will help alleviate these symptoms.

Phytochemicals have been used for thousands of years in the treatment of stress and anxiety (in addition to their use for the treatment of many other human disorders). One such plant-based therapeutic is Piper methysticum, or “kava”, which has been used as a beverage made from the root of this plant that is indigenous to the South Pacific areas of the world where it is consumed medicinally, socially, and ritualistically.

### BRIEF SUMMARY OF THE INVENTION

The present invention includes medicinal treatments for stress, anxiety and anxiety related disorders using piperine and phytochemical mixtures including piperine as the Active Pharmaceutical Ingredient(s). Embodiments of the present invention can be used to treat anxiety disorders, which are a group of mental health conditions characterized by excessive and often irrational feelings of fear, worry, and apprehension. Embodiments of the present invention can be used to treat numerous anxiety disorders, such as ones classified in a variety of ways, including generalized anxiety, panic disorder, phobias, social anxiety, obsessive-compulsive disorder and post-traumatic stress disorder (PTSD). Embodiments of the present invention can be used to treat anxiety and anxiety disorders that are triggered by stress. Embodiments of the present invention can be used to treat stress to help control any profound effects on a person's physical and mental well-being when it is a natural response to challenging or threatening situations or when it becomes chronic or overwhelming.

Embodiments of the present invention can be used to treat stress symptoms that manifest in a variety of ways. Embodiments of the present invention can be used to treat stress, which physiologically can cause individuals to experience increased heart rate, elevated blood pressure, muscle tension, and shallow breathing. In these cases, these physical symptoms are often accompanied by emotional and cognitive changes, such as irritability, anxiety, difficulty concentrating, and a sense of being overwhelmed. Embodiments of the present invention can be used to treat stress that can disrupt sleep patterns, leading to insomnia or restless nights. Embodiments of the present invention can be used to treat prolonged exposure to stress that can weaken the immune system, making individuals more susceptible to illnesses, and it may contribute to the development or exacerbation of conditions like hypertension, heart disease, and digestive disorders.

In order to discover and validate active pharmaceutical ingredients for the treatment of stress, anxiety or anxiety disorders, Gb Sciences' AI-enabled drug discovery platform (PhAROS™) was used to perform in silico convergence analysis (ISCA) and a variety of machine learning modules to look for consensus compounds in natural products from: A. Piper methysticum, B. the Piper plant family (>200 Piper spp.), or C. Gb Sciences' PhAROS™ proprietary database containing traditional medicines, species, compounds, and the health (disease) indications from Traditional Medical System data derived from 9 globally distinct TMS (not restricted to Piper spp). The best phytochemical drug candidates were selected from our in silico analysis of: A. Piper methysticum, B. the Piper plant family (>200 Piper spp.), or C. PhAROS™'s proprietary database of traditional medicines. The selected phytochemicals were tested as singles, doubles, and triples in two complementary zebrafish models of anxiety (light/dark startle response testing and thigmotaxis assays). Our PhAROS™ analysis revealed that many plants from the Piper family (which by definition lack the kavalactones that characterize P. methysticum) are used to treat anxiety or depression (the clinical indications we chose). Therefore, it seemed unlikely that the anti-anxiolytic effects would be restricted to the kavalactone compound group.

In one embodiment, a P. methysticum (kava)-developed product of the present invention provides a treatment to reduce and relieve the symptoms of stress and anxiety. The kava-developed treatment of the present invention can be administered to a person in a similar fashion as other typical medicines. This provides the user with readily available treatment relief to the major distressing symptoms of both stress and anxiety that untreated can escalate into life disrupting impacts.

In one embodiment, the kava-developed treatment product provides an anti-anxiety treatment and anti-stress treatment. In another embodiment, the treatment is delivered as an orally ingested aqueous plant extract or as plant materials delivered in a capsule form, as a ‘natural’ remedy for generalized anxiety. The orally-ingested treatment in one embodiment is a capsule the user swallows. In another embodiment, the anti-anxiety treatment product is a sublingual tablet placed under the tongue for absorption into the body. In yet another embodiment, the anti-anxiety treatment product is a liquid spray the user sprays into the oral cavity. Other methods of administration, include, but are not limited to, intravenously, intramuscularly, subcutaneously, rectally, vaginally, inhaled, intrathecally into the space around the spinal cord, buccally, ocularly, optically, nasally, by nebulization or cutaneously with a local topical or with a skin patch transdermally.

### BRIEF DESCRIPTION OF THE DRAWINGS

FIG. 1 shows a block diagram of a computational consensus analysis system used in the discovery method to investigate complex chemical compositions to determine how much of a traditional medicine herb is used, which part of the plant is used, mixture parameters, plant varieties and cultivars.

FIG. 2 shows a frequency analysis covering all piper compounds and not just that of Piper methysticum show high association of some alkaloids, terpenes, and phenylpropanoids that can be found outside of the genus to the indications of interest

FIG. 3 shows a frequency analysis where violanthin, kadsurenone, denudatin a, and xanthyletin, all belonging to the phenylpropanoid group, appear to have high association with the indications and warrants additional research.

FIG. 4 shows a frequency analysis where terpene constituents appear to have high associations with the indications of interest and can be found in many alternative plant sources found in traditional medicine systems. FIG. 5 shows larval (untreated) behavioral profile.

FIG. 5 shows larval (untreated) behavioral profile and defines the first 90 minutes in continuous light as the Baseline Exposure period, the 3 sets of intermittent pulses of 5 min dark/5 min light/5 min dark/5 min light/5 min dark/5 min light are the Light/Dark Stress Response portion of these behavioral assays.

FIG. 6 shows (A-G) concentration response profile following acute exposure to increasing concentrations of Yangonin (0.5% MeOH).

FIG. 7 shows (A-C) concentration response profile following acute exposure to increasing concentrations of Yangonin (1% MeOH).

FIG. 8 shows (A-G) concentration response profile following acute exposure to increasing concentrations of Methysticin (0.5% MeOH).

FIG. 9 shows (A-C) concentration response profile following acute exposure to increasing concentrations of Methysticin (1% MeOH).

FIG. 10 shows (A-G) concentration response profile following acute exposure to increasing concentrations of Dihydrokavain (0.5% MeOH).

FIG. 11 shows (A-C) concentration response profile following acute exposure to increasing concentrations of Dihydrokavain (1% MeOH).

FIG. 12 shows (A-G) concentration response profile following acute exposure to increasing concentrations of Dihydromethysticin (0.5% MeOH).

FIG. 13 shows (A-C) concentration response profile following acute exposure to increasing concentrations of Dihydromethysticin (1% MeOH).

FIG. 14 shows (A-G) concentration response profile following acute exposure to increasing concentrations of CBD (1% MeOH).

FIG. 15 shows (A-G) concentration response profile following acute exposure to increasing concentrations of Piperlongumine (1% MeOH).

FIG. 16 shows (A-G) concentration response profile following acute exposure to increasing concentrations of Piperine (1% MeOH).

FIG. 17 shows (A-F) concentration response profile following acute exposure to increasing concentrations of PEA (0.5% DMSO).

FIG. 18 shows (A-F) concentration response profile following acute exposure to increasing concentrations of β-Caryophyllene (0.5% DMSO).

FIG. 19 shows (A-G) concentration response profile following acute exposure to increasing concentrations of Curcumin (1% MeOH).

FIG. 20 shows (A-G) concentration response profile following acute exposure to increasing concentrations of Farnesene (1% MeOH).

FIG. 21 shows (A-F) concentration response profile following acute exposure to Bacalein (1% MeOH).

FIG. 22 shows (A-G) concentration response profile following acute exposure to increasing concentrations of Wogonin Hydrate (1% MeOH).

FIG. 23 shows (A-F) concentration response profile following acute exposure to increasing concentrations of Methyl chavicol (1% MeOH).

FIG. 24 shows (A-F) concentration response profile following acute exposure to increasing concentrations of Diazepam (control compound).

FIG. 25 shows (A-E) concentration response profile following acute exposure to increasing concentrations of Caffeine (control compound).

FIG. 26 shows concentration response profile following acute exposure to Mixture 1, concentration matched pure Yangonin/Methysticin and control treated larvae (1% MeOH).

FIG. 27 shows concentration response profile following acute exposure to Mixture 2, concentration matched pure Yangonin/Dihydromethysticin and control treated larvae. (1% MeOH).

FIG. 28 shows concentration response profile following acute exposure to Mixture 3, concentration matched pure Yangonin/Dihydrokavain and control treated larvae (1% MeOH).

FIG. 29 shows concentration response profile following acute exposure to Mixture 4, concentration matched pure Yangonin/CBD and control treated larvae (1% MeOH).

FIG. 30 shows concentration response profile following acute exposure to Mixture 5, concentration matched pure Methysticin/Dihydromethysticin and control treated larvae (1% MeOH).

FIG. 31 shows concentration response profile following acute exposure to Mixture 6, concentration matched pure Dihydrokavain/Methysticin and control treated larvae (1% MeOH).

FIG. 32 shows concentration response profile following acute exposure to Mixture 7, concentration matched pure CBD/Methysticin and control treated larvae (1% MeOH).

FIG. 33 shows concentration response profile following acute exposure to Mixture 8, concentration matched pure Dihydromethysticin/Dihydrokavain and control treated larvae (1% MeOH).

FIG. 34 shows concentration response profile following acute exposure to Mixture 9, concentration matched pure Dihydromethysticin/CBD and control treated larvae (1% MeOH).

FIG. 35 shows concentration response profile following acute exposure to Mixture 10, concentration matched pure Dihydrokavain/CBD and control treated larvae (1% MeOH).

FIG. 36 shows concentration response profile following acute exposure to multiple concentrations of Mixture 11 (Yangonin, Dihydromethysticin and Methysticin) and control treated larvae (1% MeOH).

FIG. 37 shows concentration response profile following acute exposure to multiple concentrations of Mixture 12 (Yangonin, Dihydrokavain and Methysticin) and control treated larvae (1% MeOH).

FIG. 38 shows concentration response profile following acute exposure to multiple concentrations of Mixture 13 (Yangonin, Methysticin and CBD) and control treated larvae (1% MeOH).

FIG. 39 shows concentration response profile following acute exposure to multiple concentrations of Mixture 14 (Dihydromethysticin, Dihydrokavain and Methysticin) and control treated larvae (1% MeOH).

FIG. 40 shows concentration response profile following acute exposure to multiple concentrations of Mixture 15 (CBD, Methysticin and Dihydromethysticin) and control treated larvae (1% MeOH).

FIG. 41 shows concentration response profile following acute exposure to multiple concentrations of Mixture 16 (CBD, Methysticin and Dihydromethysticin) and control treated larvae (1% MeOH).

FIG. 42 shows concentration response profile following acute exposure to multiple concentrations of ML-Mixture 1 (PEA and β-Caryophyllene) and control treated larvae (0.5% DMSO+1% MeOH).

FIG. 43 shows concentration response profile following acute exposure to multiple concentrations of ML-Mixture 1 (PEA and Curcumin) and control treated larvae (0.5% DMSO+1% MeOH).

FIG. 44 shows concentration response profile following acute exposure to ML-Mixture 3, concentration matched pure PEA and Farnesene and control treated larvae. (0.5% DMSO+1% MeOH).

FIG. 45 shows concentration response profile following acute exposure to ML-Mixture 4, concentration matched pure PEA and Wogonin hydrate and control treated larvae. (0.5% DMSO+1% MeOH).

FIG. 46 shows concentration response profile following acute exposure to ML-Mixture 5, concentration matched pure PEA and Bacalein and control treated larvae. (0.5% DMSO+1% MeOH).

FIG. 47 shows concentration response profile following acute exposure to ML-Mixture 7, concentration matched pure PEA/Methyl chavicol and control treated larvae (0.5% DMSO1% MeOH).

FIG. 48 shows concentration response profile following acute exposure to ML-Mixture 8, concentration matched pure Piperlongumine/B-Caryophyllene and control treated larvae (1% MeOH).

FIG. 49 shows concentration response profile following acute exposure to ML-Mixture 9, concentration matched pure Piperlongumine/Curcumin and control treated larvae (1% MeOH).

FIG. 50 shows concentration response profile following acute exposure to ML-Mixture 10, concentration matched pure Piperlongumine/Farnesene and control treated larvae (1% MeOH).

FIG. 51 shows concentration response profile following acute exposure to ML-Mixture 10, concentration matched pure Piperlongumine/Farnesene and control treated larvae (1% MeOH).

FIG. 52 shows concentration response profile following acute exposure to ML-Mixture 12, concentration matched pure Piperlongumine/Bacalein and control treated larvae (1% MeOH).

FIG. 53 shows concentration response profile following acute exposure to ML-Mixture 15, concentration matched pure Piperine/B-Caryophyllene and control treated larvae (1% MeOH).

FIG. 54 shows concentration response profile following acute exposure to ML-Mixture 16, concentration matched pure Piperine/Curcumin and control treated larvae (1% MeOH).

FIG. 55 shows concentration response profile following acute exposure to ML-Mixture 17, concentration matched pure Piperine/Farnesene and control treated larvae (1% MeOH).

FIG. 56 shows concentration response profile following acute exposure to ML-Mixture 18, concentration matched pure Piperine/Wogonin hydrate and control treated larvae (1% MeOH).

FIG. 57 shows concentration response profile following acute exposure to ML-Mixture 18, concentration matched pure Piperine/Bacalein and control treated larvae (1% MeOH).

FIG. 58 shows concentration response profile following acute exposure to ML-Mixture 21, concentration matched pure Piperine/Methyl chavicol and control treated larvae (1% MeOH).

FIG. 59 shows concentration response profile following acute exposure to multiple concentrations of Mixture 22 (PEA, B-Caryophyllene, and Wogonin) and control treated larvae (1% MeOH).

FIG. 60 shows concentration response profile following acute exposure to multiple concentrations of Mixture 23 (Piperine, Curcumin and Bacalein) and control treated larvae (1% MeOH).

FIG. 61A shows a Thigmotaxis Model system in a well plate for testing stress in zebrafish larvae.

FIG. 61B shows a Thigmotaxis Model with FLarvae loaded in 24 well plates 48 hpf.

FIG. 62 shows larval activity following exposure to multiple concentrations of Mixture 2.

FIG. 63 shows larval activity following exposure to multiple concentrations of Mixture 2.

FIG. 64 shows larval activity following exposure to 1 UM Diazepam.

FIG. 65 shows larval activity following exposure to 0.5 UM Diazepam.

FIG. 66 shows larval activity following exposure to 0.25 UM Diazepam.

FIG. 67 shows larval activity following exposure to multiple concentrations of Mixture 2.

FIG. 68 shows larval activity following exposure to multiple concentrations of Mixture 4.

FIG. 69 shows larval activity following exposure to multiple concentrations of ML-Mixture 15.

FIG. 70 shows larval activity following exposure to multiple concentrations of ML-Mixture 16.

### DETAILED DESCRIPTION OF THE INVENTION

In the following description, reference is made to the accompanying drawings, which form a part hereof, and in which is shown by way of illustration a specific example in which the invention may be practiced. It is to be understood that other embodiments may be utilized and structural changes may be made without departing from the scope of the present invention.

### General Overview

It should be noted that the descriptions that follow, for example, in terms of formulations for treating anxiety is described for illustrative purposes and the underlying system can apply to any number and multiple types of treatment drugs. In one embodiment of the present invention, the formulations for treating anxiety can be configured using traditional medicine plants. The formulations for treating anxiety can be configured to include a single plant compound and can be configured to include a combination of multiple components of multiple plants using the present invention.

In one embodiment, the present invention includes a drug discovery system and method of validation that integrates traditional medicine (TM) practices into western treatment approaches. The drug discovery system and method of the present invention searches traditional medicine practices that use a one disease one drug one target approach with combinations of constituent herbs in appropriate proportions to treat targeted conditions.

Western treatment approaches investigate complex disease mechanisms and the multiple, integrated biological and chemical processes to develop drugs to treat targeted conditions. Western treatment approaches include computational consensus analysis to investigate complex chemical compositions to determine how much of a traditional medicine herb is used, which part of the plant is used, mixture parameters, plant varieties and cultivars. This approach is used to create proper regulation and standardization of such ingredients, that could impact toxicity, effectiveness, and general reception to its use. The discovery method produces an understanding of the mechanism of action through the appropriate steps, identifies potential contributing bioactive components through computational consensus analysis and allows for mixtures of API as therapeutic agents.

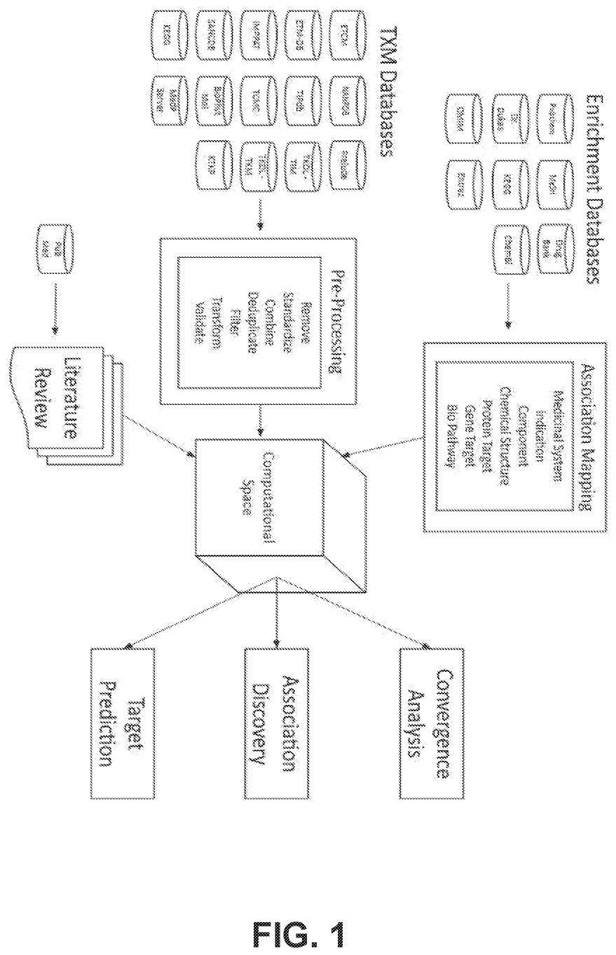

FIG. 1 shows a block diagram of a computational consensus analysis system used in the discovery method to investigate complex chemical compositions to determine how much of a traditional medicine herb is used, which part of the plant is used, mixture parameters, plant varieties and cultivars.

The discovery method determines the biological and chemical effects of said components. The discovery method uses frequency analysis and machine learning to produce an optimized combination of individual compounds for a given indication of a condition. In one embodiment, the discovery method uses tests with the kava (Piper methysticum) plant and its constituents to discover new therapeutics.

Kava is a plant from the Pacific islands used in a drink derived from the roots of Piper methysticum which is native to Oceania with few occurrences elsewhere in the world. The discovery method uses an artificial intelligence (AI) enabled drug discovery platform (PhAROS™) to use computational consensus analysis with traditional medicine data from data sources, for example the Global Biodiversity Information Facility (GBIF).

Indigenous pharmacology suggests the plant treats a variety of indications to include mood, respiratory, and venereal disorders across multiple countries. Shared and unique indications treated by Piper methysticum between countries known to use the herb in their respective traditional medicine systems. Traditional uses for Piper methysticum include skin—centipede bites, insect stings, poisonous fish stings, wound, warts, and edema; epilepsy—stiffness, convulsions, spasms; mood-anxiety disorders; calming—sedative, relaxant, pacifier, hypnotic, stress, sudorific, intoxicant, palpitations, calm; respiratory—bronchitis, sore throat, inflammation, lung disorders, chill, asthma, cold, tuberculosis, cough; reproductive—watery vaginal discharge, womb disorders, uterine disorders, gonorrhea, venereal disorders, menstrual irregularities, aphrodisiac, urogenital-antiseptic; pain—rheumatism, myalgia, headache, toothache, backache, stomachache, joint pain, narcotic, pain; and other-filariasis, debility, tonic, elephantiasis, yaws, kidney disorders, stimulant, cystitis, weightless. The majority of TM usage comes from Oceania with some incorporation into medicinal systems in parts of Asia and the Middle East

Kava is often used for its anxiolytic, relaxant, and analgesic effects confirmed through various studies attributed to the plant's kavalactones, lipophilic resinous compounds unique to Piper methysticum. The mechanism of action appears to include blockage of voltage gated sodium and calcium ion channels, enhanced binding to GABAa receptors, reduced neuronal reuptake of excitatory transmitors, and suppression of GABAa antagonizers like eicosanoid thromboxane A2. In one embodiment the discovery method uses computational consensus analysis of chemical components, ingredient organisms, and indications to identify the mechanisms of action.

The chemical composition of Piper methysticum includes alkaloids, benzenoids, pyridines, terpenes, cinnamic acids, flavonoids, kavalactones, other phenylpropanoids, chalcones, and other chemical compositions. There are 189 recorded Piper methysticum compounds, of which there are 11 alkaloids, 33 benzenoids, 4 pyridines, 37 terpenes, 24 cinnamic acids, 8 flavonoids, 36 kavalactones, 2 miscellaneous phenylpropanoids, 12 chalcones, and 22 miscellaneous compounds. Kavalactones and chalcones are the most extensively studied of all the compounds of Piper methysticum.

In one embodiment, frequency analysis was conducted on the known Piper methysticum compounds also found in other species (this does not include most kavalactones and chalcones which are unique to the plant). Within the compiled dataset, the number of formulas with the compound was identified and the percentage of said formulas treating each indication of interest was calculated. Canadine, anisole, camphor, and 1-cinnamoylpyrrolidine appear to stand out as non-kavalactone components associated with not just one indication of interest, but all four. Piper methysticum is a unique plant in chemical composition and therapeutic effect.

Quite a few of the species in the Piper plant family are associated with anxiety, pain, sleep, and epilepsy disorders. Piper borbonense, Piper guineense, Piper Kadsura, Piper longum, Piper methysticum, Piper nigrum, and Piper umbellatum are plants within the Piper family from which herbal ingredients have been used in formulas treating all four indications within the compiled traditional medicine dataset. 10 additional species are associated with 2 or more indications. Piper nigrum, Piper longum, and Piper kadsura appear to be used more often in association with the indications of interest than other species. Pain makes up the bulk of indications treated by Piper nigrum and Piper longum both of which are used to treat a wide variety of indications across the dataset. By percentage, Piper methysticum, Piper kadsura, and Piper borbonense appear to be used specifically for mood, pain, epilepsy, and sleep disorders. Species that are associated with the indications in traditional medicines, but that do not appear to have a direct relationship with the indications in current literature as far as mechanism of action are concerned include: Piper acutifolium, P. angustifolium, P. attenuatum, P. barbatum, P. boehmeriifolium, P. borbonense, P. carpunya, P. cavalcantei, P. corcovadensis, P. darienense, P. dennisii, P. elongatum, P. excelsum, P. fimbriulatum, P. fragile, P. hainanense, P. hancei, P. jacquemontianum, P. klotzschianum, P. mullesua, P. obliquum, P. ovatum, P. peltatum, P. piscatorum, P. pyrifolium, P. sylvestre, and P. tutuilae. Frequency analysis of known Piper compounds within more than 5 txm formulas with respect to mood, sleep, epilepsy, and pain formulas were analyzed.

In one embodiment, the original frequency analysis was extended to cover chemical components of all Piper species. According to the compiled dataset, the various Piper species share quite a few bioactive alkaloids, terpenes, and phenylpropanoids. Piper nigrum and Piper cubeba share the most known constituents at 333 compounds, whereas P. brachystachyum, P. chaba, P. maclurei, P. patulum, and P. sanctum have the fewest known compounds and do not share any known compounds with other species in the genus. The most extensively studied species is Piper nigrum with over 1000 known constituents and derivatives.

In one embodiment, frequency analysis covering all piper compounds and not just that of Piper methysticum show high association of some alkaloids, terpenes, and phenylpropanoids that can be found outside of the genus, which are very relevant to the indications of interest and elucidate new potential therapeutic options and alternatives to kava.

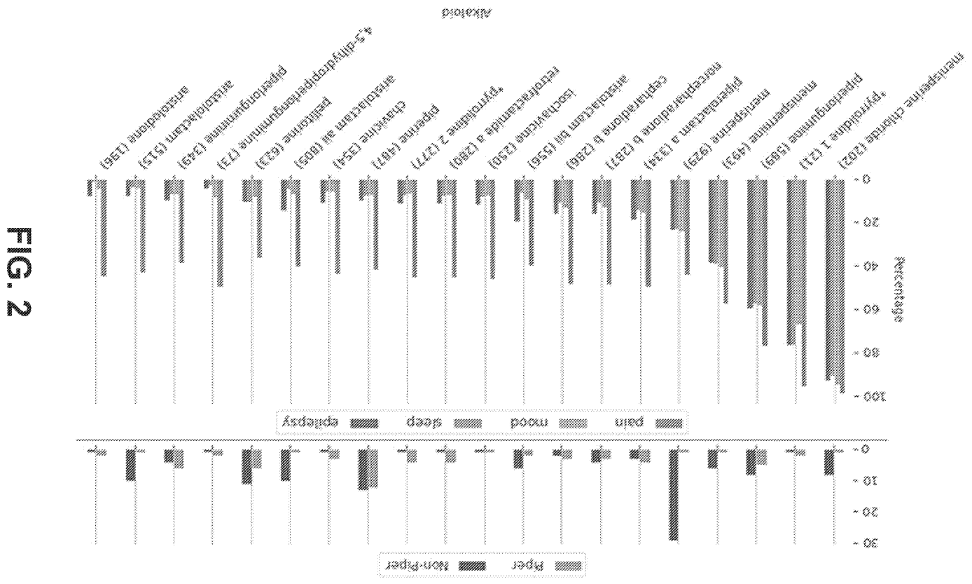

FIG. 2 shows a frequency analysis covering all Piper compounds and not just that of Piper methysticum that show high association of some alkaloids, terpenes, and phenylpropanoids that can be found outside of the genus to the indications of interest. Over 80% (n=202) of traditional medicine formulas with menisperine chloride treat all indications of interest, for example *pyrrolidine 1=1-[(2e,4e,8e)-9-(3,4-methylenedioxyphenyl)-2,4,8-nonatrienoyl]pyrrolidine and *pyrrolidine 2=1-[(2e,8e)-9-(3,4-methylenedioxyphenyl)-2,8-nonadienoyl]pyrrolidine.

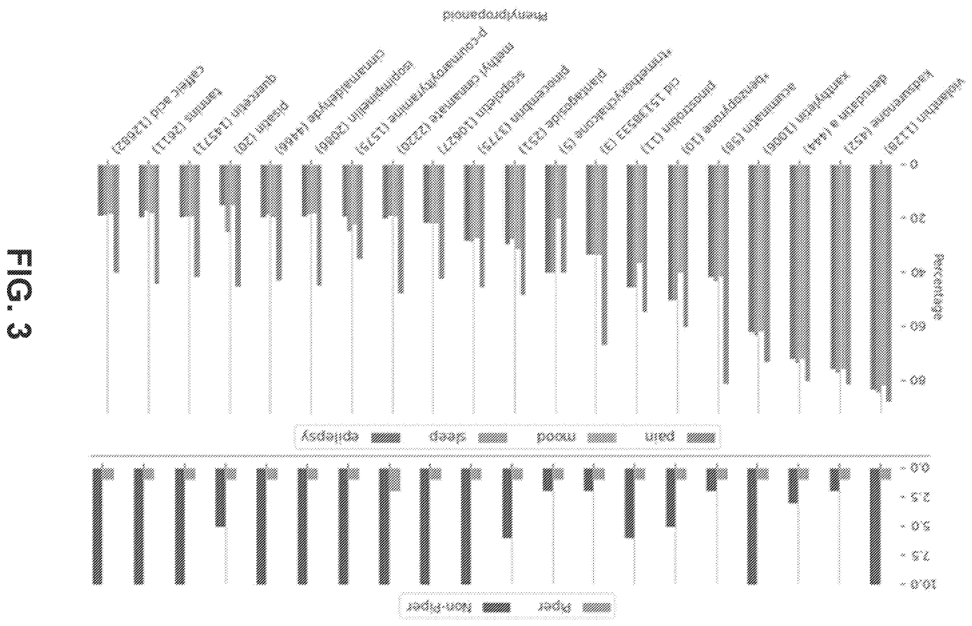

FIG. 3 shows a frequency analysis where violanthin, kadsurenone, denudatin a, and xanthyletin, all belonging to the phenylpropanoid group, appear to have high association with the indications and warrants additional research. The discovery method also found Violanthin, kadsurenone, denudatin a, and xanthyletin, all belonging to the phenylpropanoid group, appear to have high association with the indications, for example *benzopyrone=(s)-2,3-dihydro-5-hydroxy-7-methoxy-2-phenyl-4-benzopyrone and *trimethoxychalcone=2′-hydroxy-4,4′,6′-trimethoxychalcone.

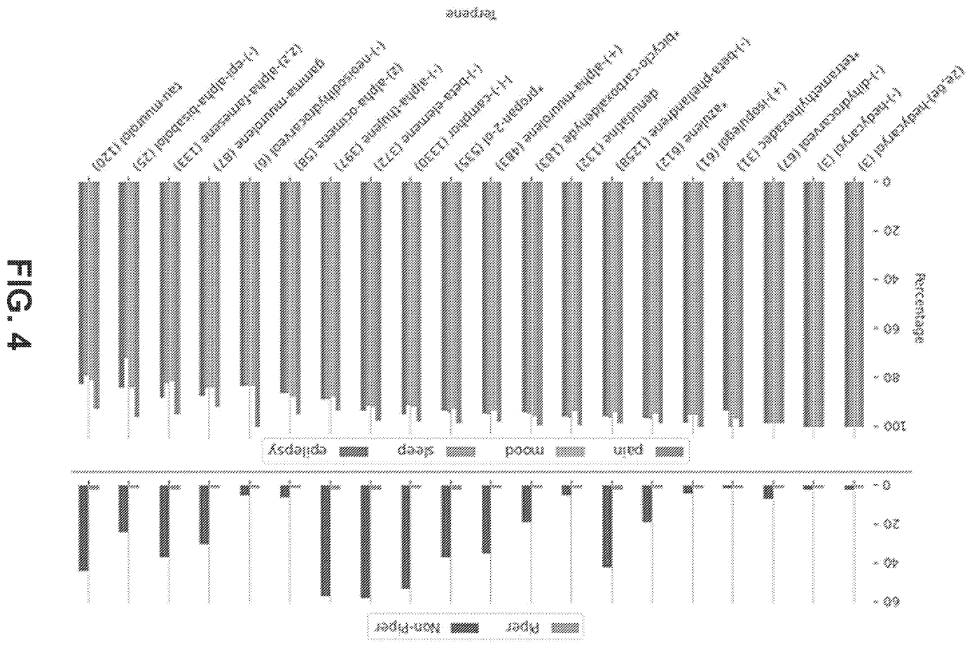

FIG. 4 shows a frequency analysis where terpene constituents appear to have high associations with the indications of interest and can be found in many alternative plant sources found in traditional medicine systems. The discovery method also found terpene constituents appear to have high associations with the indications of interest and can be found in many alternative plant sources found in traditional medicine systems, for example *tetramethylhexadec=(e,7s, 11r)-3,7,11,15-tetramethylhexadec-2-en-1-ol and *azulene=azulene, 1,2,3,4,5,6,7,8-octahydro-1,4-dimethyl-7-(1-methylethenyl)-, (1s,4s,7r)-.

The discovery method also used a PubMed search of each Piper species with each indication of interest and their respective keywords was conducted. Of the compounds within the Piper species, linalool, p-cymene, alpha-terpineol, and beta-caryophyllene are terpenes within 12K+ formulas and some association to the indication of interest. 4 alkaloids: piperolactam a, norcepharadione b, piperine, and piperolactam care captured in the scatterplot. Of the 25, Piper attenuatum, Piper mullesua, Piper obliquum, and Piper hancei each had association to the indications of interest. In one embodiment, the zebrafish experiments validate the beneficial effect of Piper methysticum with conditions of interest including stress and mood-anxiety disorders.

The discovery method also searched traditional medicine system datasets via site download options or web data extraction of a number of data sites. Protein targets were matched to compounds showing identifiers and bioassays. Exploratory data analysis was conducted additionally from other sites.

The discovery method revealed Piper methysticum has over one hundred different chemotypes and cultivars. All herbal medicine factors of consistency and quality of cultivation are a priority in its use as a therapeutic. Individual components and bioactive compounds in a particular herb's usage when treating specific indications are identified.

Identifying alternative sources for the bioactive compounds is making a potential therapeutic combination more widely accessible. The computational consensus is used to filter down to potential therapeutics for any given indication. The Piper genus is used for a wide variety of indications across the world; non-methysticum plants are also used for the selected indications and the analytics can be applied to any one of them and it can be adapted to any given parameter whether that be indication, genus, species, or compound.

The present invention is a kava plant (Piper methysticum) developed product for treating stress, anxiety and anxiety related disorders. The kava-developed treatment product provides a blissful experience and is a potent anti-anxiety and anti-stress treatment without psychoactive side effects.

The invention includes a process of formulating a pharmaceutical composition for treating stress, anxiety and anxiety-related disorders in a human. The formulation process includes investigation of plants and their constituent compositions for potential beneficial effects to stress and anxiety control. This investigative search produced results identifying piperine as a compound from plants in the Piper plant family with great potential to alleviate the targeted conditions of anxiety and mood disorders.

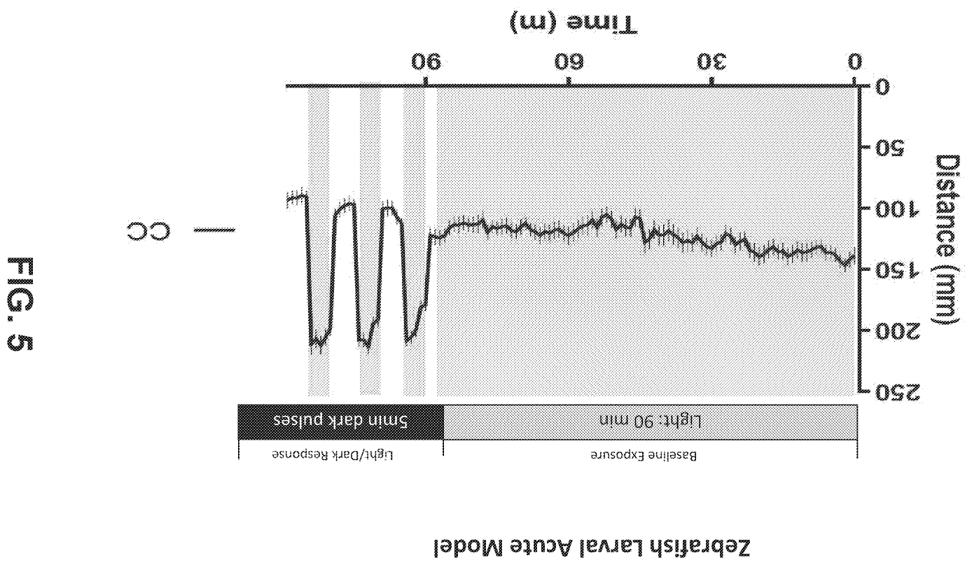

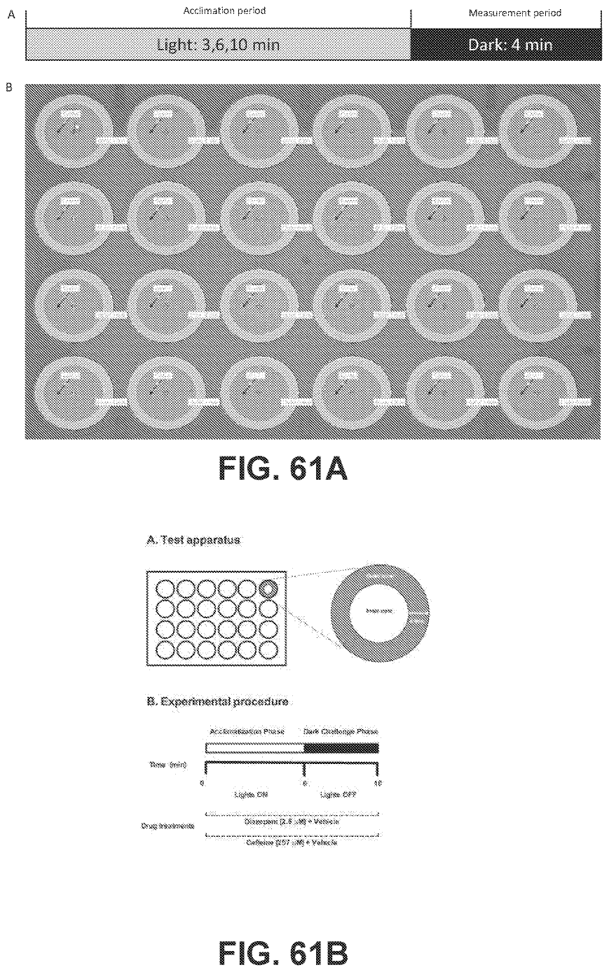

The validation process for the phytochemicals derived from Piper methysticum, other Piper plant family members, and non-Piper medicinal plants and fungi includes testing the selected phytochemical drug candidates as singles, doubles, and triples in two complementary zebrafish models of stress and anxiety. These two complementary assays are the Light/Dark Stress Response test that is illustrated in FIG. 5 (after the 90 min study of the effect of the compounds and mixtures on Baseline Activity) and the Thigmotaxis assay, as illustrated in FIG. 61A and FIG. 61B, which is also known as the wall flower effect, like the social anxiety of humans. Zebrafish larvae show a clear and distinct pattern of swimming in response to rapidly alternating light and dark conditions in the Light/Dark Response testing, which trigger a flight or fight response and darting behavior indicative of great stress and anxiety. The Light/Dark testing uses a control group to automatically track, using an automatic tracking device, the larval behavior in response to dark to light conditions. The thigmotaxis assay also uses automatic tracking and appropriate controls. In multiple embodiments additional Zebrafish larvae are given various concentrations of the selected phytochemicals from Table 1 for tracking Light/Dark Responses or Thigmotactic behavioral changes in the treated zebrafish larvae. Table 2A and Table 2B summarize the results from both assays when the individual phytochemicals are exposed to the: Baseline Activity (90 minutes in the light), Light/Dark Stress Response (5 min alternating pulses of dark and light for 30 min.), and Thigmotaxis Activity (% of time in the light center of the chamber (anxiety inducing) versus % of time near the dark (soothing) wall).

Additionally, testing is done with mixtures of these same selected phytochemicals from P. methysticum and other plants selected by our in silico analyses. The testing results are analyzed using artificial intelligence systems to rank effectiveness in reducing the Light/Dark response and thigmotactic behavior for the

TABLE 1
Results of In Silico Analyses_Phytochemicals for the Treatment of Stress or
Anxiety
Type A: Piper methysticum

| | | Molecular | CAS | | Approx. Dose (cell or |
|---|---|---|---|---|---|
| Compounds | Type | Weight | number | Potential Target(s) | animal experiments) |
| yangonin | pyranone | 258.27 | 500-62-9 | CBR/MAOs inhibitor | MAO-A1.29 μM, |
| | | | | | MAO-B 0.085 μM |
| R-(+)-methysticin | lactone/pyrone | 274.27 | 495-85-2 | MAO inhibitor, Ca2+ | MAO-A 8.12 μM, |
| | | | | channels, GABA A Rec, | MAO-B 0.5 μM |
| (+)-dihydro | lactone/pyrone | 276.28 | 19902-91-1 | Block Norepinephron | MAO-A 23.2 μM, |
| methysticin | | | | | MAO-B 0.855 μM |
| (S)-(4)-7,8- | lactone/pyrone | 232.28 | 587-63-3 | | MAO-A none, |
| dihydrokavain | | | | | MAO-B 8.23 μM |

Type B: Piper Plant Family

| Compounds | Type | MW | CAS number | Potential Target(s) | Approx. Dose |
|---|---|---|---|---|---|
| piperlongumine | alkaloid | 317.34 | 20069-09-4 | inhibits NO & NFκB | 5 μM in anti-inflammatory |
| | | | | pathways | cell assays |
| piperine | alkaloid | 285.34 | 94-62-2 | GABAergic Rec, NOS | 5, 10, and 20 mg/kg (ip) |
| | | | | | rodents |
| beta | terpene | 204.35 | 87-44-5 | CBR/GABAergic Rec | 50 mg/kg animal study |
| caryophyllene | | | | | |
| (BCP) | | | | | |
| farnesene | terpene | 204.35 | isomers | CBR? | 20 mg/kg oil, low % |
| | | | | | farnesene |

Type C: Transcultural Compounds (PhAROS database of global traditional medical systems)

| Compounds | Type | MW | CAS number | Potential Target(s) | Approx. Dose |
|---|---|---|---|---|---|
| cannabidiol | cannabinoid | 314.46 | 13956-29-1 | CBR/TRP channels | 20 mg/kg animal study |
| (CBD) | | | | | |
| N-palmitoyl | alkaloid/FAA | 299.49 | 544-31-0 | GPR55; CBR, PPAR-α | 50 mg/kg animal study |
| ethanolamide | | | | | |
| (PEA) | | | | | |
| wogonin | flavonoid | 284.26 | 632-85-9 | GABAergic Rec, ROS | 1, 10, 20 mg/kg ip rat |
| (hydrate) | | | | pathways, NFκB | |
| bacalein | flavonoid | 270.24 | 491-67-8 | COX-2 inhibitor, reduced | 10, 20, and 40 mg/kg oral |
| | | | | PGE2, ERK phos, BDNF | rat |
| curcumin | terpene | 368.38 | 458-37-7 | MAO inhibitor | MAOA 9.01 μM, MAOB |
| | | | | | 2.55 μM |
| methyl chavicol | phenylpropoloid | 148.2 | 140-67-0 | anti-inflammatory, anti- | 10, 20, 20 mg/kg ip mice |
| (4-allylanisole) | | | | oxidant | |

Abbreviations:
CBR = cannabinoid receptors (CB1 and/or CB2R);
MAO = Monoamine Oxidase;
Ca+ channels = calcium dependent channels;
GABA = gamma-aminobutyric acid;
NO = Nitric oxide;
NFκB = nuclear factor kappa-light-chain-enhancer of activated B cells;
GABAergic = pertains to or affects the neurotransmitter gamma-aminobutyric acid;
NOS = Nitric oxide synthases;
TRP channels = Transient Receptor Potential channels;
GPR55 = G-protein coupled receptor 55;
PPAR-α = peroxisome proliferator-activated receptor alpha;
ROS = reactive oxygen species pathways;
COX-2 = cyclooxygenase-2;
PGE2 = cyclooxygenase-2;
ERK= extracellular signal-regulated kinase;
BDNF = brain-derived neurotrophic factor

differing concentrations of the phytochemicals of Piper methysticum, other plants from the Piper family and non-Piper plants and fungi with other compounds in differing concentrations. The results analysis rankings are used to formulate effective pharmaceutical composition dosage concentrations of the Piper methysticum alone and those in combinations of other compounds with the Piper methysticum for the treatment of stress, anxiety and anxiety-related disorders in a human.

In order to discover active pharmaceutical ingredients for the treatment of stress, anxiety or depression, an artificial intelligence (AI) enabled drug discovery In order to discover active pharmaceutical ingredients for the treatment of stress, anxiety or depression, an artificial intelligence (AI) enabled drug discovery platform was used to perform in silico convergence analysis (ISCA) and a variety of machine learning modules to look for consensus compounds in natural products from: A Piper methysticum (kava), B. within the Piper plant family (>200 Piper species pluralis (spp).), or C. the A1 enabled drug discovery platform database containing traditional medicines, species, compounds, and the health (disease) indications derived from traditional medical system data derived from distinct traditional medical systems that are not restricted to Piper spp.

The A1 enabled drug discovery platform database search data was evaluated to select the best drug candidates from: A Piper methysticum, B. within the Piper plant family (>200 Piper spp.), or C. traditional medicines to begin testing analytical profile index (API) singles, doubles, and triples in two complementary zebrafish models of anxiety light/dark startle response testing and thigmotaxis assays. The results of this further analysis are presented as Table 1.

The artificial intelligence (AI) enabled drug discovery platform machine learning modules analysis revealed that many plants from the Piper family, which lack kavalactones that characterize Piper methysticum, are used to treat anxiety or depression the clinical indications chosen. Therefore, it seemed unlikely that the anti-anxiolytic effects would be restricted in this testing to the Piper methysticum kavalactone compound group selected for the targeted condition anxiety and related anxiety disorders. The in silico analyses and animal testing models have confirmed the potential for certain phytochemicals extracted from the Piper methysticum plant, including the kavalactone compound group, are effective in relieving stress or anxiety.

The present invention includes a process to discover new pharmaceutical medicines for the treatment of stress, anxiety and anxiety-related disorders in a human. The phytochemical drug candidates derived from the in silico analyses are presented in Table 1. The preclinical testing included the effects of these 14 individual phytochemicals and 300 ratio-controlled mixtures of these phytochemicals in assays measuring the behavioral stress response patterns of zebrafish larvae. Larval behavior is assessed with at least one functional assay of stress or anxiety following acute exposure to each phytochemical sample formulated. Stereotypical basal activity patterns of zebrafish will be assessed to determine the potential side effects of the phytochemical samples in a concentration dependent manner for each compound on two zebrafish models of stress and anxiety: 1) the Light/Dark Stress Response (5 min alternating pulses of light and dark) in small to med well plates (96-48 well plates), and 2) Thigmotaxis assays in large well plates with 50% of the swimming surface colored dark around the margins of the well in large (24-12 well plates) will be assessed for their efficacy in testing the phytochemicals.

The efficacy of kava extracts cannot be reduced to a single ingredient, but it has also been found that one of the compounds in the plant extract may cause liver toxicity. The in silico analyses used herein found substitutes that are analogous molecules from different plants in the same family, and we found a couple of different natural “biosimilars”, which were also tested. Data was generated in two complementary animal models of stress and anxiety demonstrating that some minimal essential mixture (MEM) formulations reduced anxiety with statistical significance. This data set also demonstrated significant synergy among the ingredients. The magnitude of the anti-anxiolytic activity of the individual ingredients, in some cases, is tiny compared with the

TABLE 2A
Results of API used singly in zebrafish assays of Stress & Anxiety

| | | | Baseline | | | Light/Dark Stress | | | Thigmotaxis | | | Anti- |
|---|---|---|---|---|---|---|---|---|---|---|---|---|
| | | | Exposure | | | Response | | | Model | | | Anxiety |
| Compounds | Type | Potential Target(s) | Low | Med | High | Low | Med | High | Low | Med | High | Potential? |

Type A: Piper methysticum

| | | | | | | | | | |
|---|---|---|---|---|---|---|---|---|---|
| yangonin | pyranone | CBR/MAOs inhibitor | + | + | + | −− | −− | −− | Yes |
| R-(+)-methysticin | lactone/pyrone | MAO inhibitor, Ca2+ | + | | −− | | | | No |
| (+)-dihydro | lactone/pyrone | channels, GABA A Rec, | + | −− | −− | | −− | −− | Yes |
| methysticin | | Block Norepinephron | | | | | | | |
| (S)-(+)-7,8- | lactone/pyrone | | + | −− | −− | | | | Yes |
| dihydrokavain | | | | | | | | | |

Type B: Piper Plant Family

| | | | | | | | | | | | |
|---|---|---|---|---|---|---|---|---|---|---|---|
| Piperlongumine | alkaloid | inhibits NO & NFkB | | + | | + | | −− | −− | −− | Yes |
| | | pathways | | | | | | | | | |
| piperine | alkaloid | GABAergic Rec, NOS | −− | | + | | −− | −− | | −− | Yes |
| beta | terpene | CBR/GABAergic Rec | + | + | + | + | | −− | | | No |
| caryophyllene | | | | | | | | | | | |
| farnesene | terpene | CBR? | | + | + | | | −− | | | Yes |

Type C: Transcultural Compounds (PhAROS database of global traditional medical systems)

| | | | | | | | | | | |
|---|---|---|---|---|---|---|---|---|---|---|
| cannabidiol | cannabinoid | CBR | + | −− | −− | + | −− | −− | −− | Yes |
| (CBD) | | | | | | | | | | |
| N-palmitoyl | Alkaloid/FAA | GPR55; CBR, PPAR-α | | + | | | + | + | | No |
| ethanolamide | | | | | | | | | | |
| (PEA) | | | | | | | | | | |
| Wogonin | flavonoid | GABAergic Rec, ROS | | + | + | | | | | No |
| (hydrate) | | pathways, NFkB | | | | | | | | |
| bacalein | flavonoid | COX-2 inhibitor, | | + | −− | | −− | −− | | Yes |
| | | reduced PG E2, ERK | | | | | | | | |
| | | phos, BDNF | | | | | | | | |
| curcumin | terpene | MAO inhibitor | | + | + | | | −− | | No |
| methyl | phenylpropoloid | anti-inflammatory, | | −− | −− | | | + | | No |
| chavicol (4- | | anti-oxidant | | | | | | | | |
| allylanisole) | | | | | | | | | | |

Abbreviations: “+” indicates statistically significant increase; “−−” = statistically significant decrease; CBR = cannabinoid receptors (CB1 and/or CB2R); MAO = Monoamine Oxidase; Ca+ channels = calcium dependent channels; GABA = gamma-aminobutyric acid; NO = Nitric oxide; NFkB:: nuclear factor kappa-light-chain-enhancer of activated B cells; GABAergic = pertains to or affects the neurotransmitter gamma-aminobutyric acid; NOS = Nitric oxide synthases; TRP channels = Transient Receptor Potential channels; GPR55 = G-protein coupled receptor 55; PPAR-a = peroxisome proliferator-activated receptor alpha; ROS = reactive oxygen species pathways; COX-2 = cyclooxygenase-2; PGE2 = cyclooxygenase-2; ERK = extracellular signal-regulated kinase; BDNF = brain-derived neurotrophic factor

anti-anxiolytic activity of some phytochemical mixtures in these preclinical studies. Therefore, the efficacy of the mixture is likely more than an additive effect of the efficacies of the individual ingredients.

The active pharmaceutical ingredients within Piper methysticum are not well characterized for use in pharmaceutical medicines. Kavalactones are presumed to be the active ingredients in kava (Piper methysticum). This was validated in the two zebrafish models tested. However, compounds that were not found in P. methysticum were also found to have significant anti-anxiolytic activity.

Because the in silico analyses were effective at prescreening the phytochemical compounds for use in treating stress and anxiety, the testing revealed a high percentage of individual compounds and even higher percentages of phytochemical mixtures demonstrating the statistically significant reduction in stress and anxiety in the zebrafish models tested. As in Table 2A & Table 2B, a total of 7 out of the 14 phytochemicals selected by the in-silico analyses demonstrated anti-anxiolytic activity when used as single ingredient therapeutics. 2 out of 4 compounds from Piper methysticum, 3 out of 4 compounds from the Piper plant family that are not Piper methysticum, and 2 out of 6 compounds from the A1 enabled drug discovery platform database search data transcultural database demonstrated anti-anxiolytic activity when used as single ingredient therapeutics.

Certain ingredients from the kava plant (Piper methysticum) will be developed as a therapeutic product for stress and anxiety. Certain ingredients from the Piper plant family will also be developed as a therapeutic product for stress and anxiety.

Testing was also performed on pairs or triplets of the phytochemicals. As summarized in Tables 3A & 3B, a total of 60 out of the 229 ratio-controlled phytochemical mixtures that were tested in pairs demonstrated anti-anxiolytic activity, for a 26.2% success rate. In addition, 50 out of the 72 ratio-controlled phytochemical mixtures demonstrated anti-anxiolytic activity when used in triplets, for a 69.4% success rate. When the compounds in the mixtures were derived from Piper methysticum plus

TABLE 2B
Phytochemical effects on baseline activity impacts therapeutic utility

| Phytochemical | | Light/Dark Stress Response (5 min | Therapeutic |
|---|---|---|---|
| (vehicle) | baseline Activity (90 min light) | light/dark pulses) | Potential |
| Yangonin (0.5% | baseline activity is close to controls | stress response is reduced | Yes |
| MeOH) | (slight increase initially) | | |
| Yangonin (1% | baseline activity is close to controls | stress response is reduced | Yes |
| MeOH) | (slight increase initially) | | |
| Methysticin (0.5% | baseline activity levels reduced | no reduction in stress response | No |
| MeOH) | (evidence of sedation) | | |
| Methysticin (1% | baseline activity levels reduced | stress response is reduced, but high | No, sedative effect |
| MeOH) | (evidence of sedation) | sedation levels | |
| Dihydromysticin | baseline activity is close to controls | stress response is reduced | Yes |
| (0.5% MeOH) | (slight increase initially) | | |
| Dihydromysticin | baseline activity levels reduced | stress response is reduced, but evidence | Yes, but sedation is a |
| (1% MeOH) | (evidence of sedation) | of sedation | factor |
| Dihydrokavain | baseline activity is close to controls | no reduction in stress response | No |
| (0.5% MeOH) | (slight increase initially) | | |
| Dihydrokavain | baseline activity levels reduced | stress response trending down, but high | No |
| (1% MeOH) | (evidence of sedation) | sedation | |
| Piperlongumine | baseline activity not like controls | stress response is reduced | Yes, but sedation is a |
| (1% MeOH) | (increased at low concentrations, | | factor in active range |
| | sedating at higher concentrations) | | |
| Piperine (1% | baseline activ ity is very close to | stress response is reduced | YES-top pick |
| MeOH) | controls | | |
| beta- | baseline activity not like controls | stress response reduced in highest | No |
| Caryophyllene | (increased at all concentrations) | conc., but baseline activity is concerning | |
| (1% MeOH) | | | |
| Farnesene (1% | baseline activity is very close to | no reduction in stress response | No |
| MeOH) | controls | | |
| Cannabidiol (1% | baseline activity not like controls | stress response is reduced | Yes, but sedation is a |
| MeOH) | (increased at low concentrations, | | factor in active range |
| | sedating at higher concentrations) | | |
| N-palmitoyl | baseline activity not like controls | stress response sometimes increased | No |
| ethanolamide | (increased at all concentrations) | | |
| (PEA) | | | |
| Wogonin | baseline activity not like controls | no reduction in stress response | No |
| (hydrate) (1% | (increased at med/high | | |
| MeOH) | concentrations) | | |
| Bacalein (1% | baseline activity not like controls | stress response is reduced, but sedation | Yes, but sedation is a |
| MeOH) | (increased at low concentrations, | observed | factor in active range |
| | sedating at higher concentrations) | | |
| Curcumin (1% | baseline activity not like controls | stress response reduced in highest | No, sedative effect |
| MeOH) | (increased at med/high | conc., but baseline activity is concerning | |
| | concentrations) | | |
| methyl Chavicol | baseline activity not like controls | no reduction in stress response, some | No |
| (1% MeOH) | (decreased at high concentrations) | increase | |

CBD, 73 out of the 158 ratio-controlled phytochemical mixtures that were tested demonstrated anti-anxiolytic activity, for a 46.2% success rate.

When the compounds in the mixtures were derived from other plants in general and other members of the Piper plant family, 37 out of the 143 ratio-controlled phytochemical mixtures that were tested demonstrated anti-anxiolytic activity, for a 25.9% success rate. When the compounds in the mixtures were pairs of compounds derived from Piper methysticum, the success rate was 30.4%. When the compounds in the mixtures were triplets of compounds derived from Piper methysticum, the success rate was 75.0%. When the compounds in the mixtures were pairs of compounds derived from other plants in general and other members of the Piper plant family, the success rate was 25.0%. When the compounds in the mixtures were triplets of compounds derived from other plants in general and other members of the Piper plant family, the success rate was 50.0%.

The active phytochemical compounds that were tested that were derived from within Piper methysticum were more effective in simple mixtures of two or three compounds than when used individually as therapeutics. The triplet formulations of the Piper methysticum derived mixtures (plus some with CBD) had a success rate of 75.0% in reducing stress or anxiety in the zebrafish models.

The following ratio-controlled phytochemical mixtures of ingredients from within P. methysticum provided promising anti-anxiolytic results:
5 μM yangonin: 10 UM methysticin.
10 UM yangonin: 10 UM methysticin
10 UM yangonin: 5 μM dihydromethysticin
10 μM yangonin: 10 UM dihydromethysticin.

The following ratio-controlled phytochemical mixture with ingredients that were not from P. methysticum was also very effective: 3 μM piperine: 10 UM beta-caryophyllene and 5 UM piperine: 10 μM beta-caryophyllene.

TABLE 3A
Phytochemical Mixtures derived from Piper methysticum and/or Cannabidiol (CBD)

| GbS | Statistically Significant | yangonin | methysticin | dihydro | dihydrokavain′ | cannabidiol |
|---|---|---|---|---|---|---|
| Mixtures | Decrease in Stress Model? | (Y) | (M) | methysticin (DM) | (DK) | (CBD) |
| GBS.001 | Yes, 2 out of 9 ratios | X | X | | | |
| CBS.002 | Yes, 5 out of 9 ratios | X | | X | | |
| GBS.003 | No | X | | | X | |
| GBS.004 | Yes, 3 out of 12 ratios | X | | | | X |
| GBS.005 | Yes, 5 out of 9 ratios | | X | X | | |
| GBS.006 | Yes, 2 out of 9 ratios | | X | | X | |
| GBS.007 | Yes, 2 out of 12 ratios | | X | | | X |
| GBS.008 | Yes, 3 out of 9 ratios | | | X | X | |
| GBS.009 | Yes, 4 out of 12 ratios | | | X | | X |
| GBS.010 | Yes, 5 out of 12 ratios | | | | X | X |
| GBS.OII | Yes, 8 out of 8 ratios | X | X | X | | |
| GBS.012 | Yes, 8 out of 8 ratios | X | X | | X | |
| GBS.013 | Yes, 6 out of 8 ratios | X | X | | | 1 X |
| CBS.014 | Yes, 6 out of 8 ratios | | X | X | X | |
| GBS.015 | Yes, 9 out of 12 ratios | | X | X | | X |
| GBS.016 | Yes, 5 out of 12 ratios | | | X | X | X |

In one embodiment, some of the active compounds from the Piper plant family (not Piper methysticum) were able to demonstrate significant anti-anxiolytic activity as single ingredient therapies. The pharmacokinetics of piperine acting alone had positive results and is very promising as a therapeutic when used and in this concentration range. Also, there was virtually no effect on the baseline activity, which is clinically desirable. It is notable that piperine is found broadly in plants in the Piper family, but piperine is not a kavalactone.

Table 3A and Table 3B represent summaries of the potential drug combinations that were discovered with the discovery drug platform discussed above.

TABLE 3B
Mixtures of Phytochemical Compounds (not from P. methysticum)

| | Statistically | | | | | | | | | |
|---|---|---|---|---|---|---|---|---|---|---|
| | Significant | N-palmitoyl | piper- | | β- | | | | | |
| GbS | Decrease in | ethanolamide | longumine | piperine | caryophyllene | curcumin | farnesene | wogonin | bacalein | chavicol |
| Mixtures | Stress Model? | (PEA) | (PL) | (P) | (BC) | (C) | (F) | (W) | (B) | (CV) |
| GBS.ML001 | No | X | | | X | | | | | |
| GBS.ML002 | No | X | | | | X | | | | |
| GBS.ML003 | No | X | | | | | X | | | |
| GBS.M 1004 | No | X | | | | | | X | | |
| GBS.ML005 | No | I X | | | | | | | X | |
| GBS.ML007 | No | X | | | | | | | | X |
| GBS.ML008 | Yes, 5 out of 9 | | X | | X | | | | | |
| | ratios | | | | | | | | | |
| GBS.ML009 | Yes, 4 out of 9 | | X | | | X | | | | |
| | ratios | | | | | | | | | |
| GBS.M L010 | No | | X | | | | X | | | |
| GBS.ML011 | Yes, 2 out of 9 | | X | | | | | X | | |
| | ratios | | | | | | | | | |
| GBS.ML012 | Yes, 3 out of 9 | | X | | | | | | X | |
| | ratios | | | | | | | | | |
| GBS.M L014 | No | | X | | | | | | | X |
| GBS.ML015 | Yes, 5 out of 9 | | | X | X | | | | | |
| | ratios | | | | | | | | | |
| GBS.ML016 | Yes, 5 out of 9 | | | X | | X | | | | |
| | ratios | | | | | | | | | |
| GBS.ML017 | Yes, 2 out of 9 | | | X | | | X | | | |
| | ratios | | | | | | | | | |
| GBS.ML018 | No | | | X | | | | X | | |
| GBS.ML019 | Yes, 3 out of 9 | | | X | | | | | X | |
| | ratios | | | | | | | | | |
| GBS.ML021 | No | | | X | | | | | | X |
| GBS.ML022 | Yes, 4 out of 8 | X | | | X | | | X | | |
| | ratios | | | | | | | | | |
| GBS.ML023 | Yes, 4 out of 8 | | X | | | X | | | X | |
| | ratios | | | | | | | | | |

### Testing Methods and Results

The discovery methods of the present invention include testing the effects of up to 20 phytochemicals and 40 mixtures of the phytochemicals on behavioral stress response patterns of zebrafish larvae. Larval behavior was assessed with an automated video tracking system following acute exposure to each phytochemical sample. Stereotypical basal activity patterns of larvae were assessed to determine the concentration dependent effect of each compound. A zebrafish model of thigmotaxis in large well plates (24-12 well plates) was assessed for their efficacy in testing the phytochemicals. In the discovery method, fertilized eggs were kept on a re-circulating system in baskets until use at 120 hpf (5 days). At 120 hpf, larvae were loaded into 48 well plates in buffered fish media and acclimated to the plate in a heated light incubator for 2 hours. Larvae were acutely exposed with drug or controls at a 10× concentration and the plate was quickly transferred to a behavioral tracking system. Acute behavioral run was 90 minutes (baseline activity) in the light followed by alternating 5 minute bins (total 2 hours). In the discovery method, each concentration and control was duplicated, at the minimum, with n=12, N=2-3. The average distance moved was binned into 1 minute frames that produced a 2 hour behavioral profile.

FIG. 5 shows larval (untreated) behavioral profile over a 2-hour time frame consisting of 90-minute exposure to light followed by 30 minutes of alternating 5 min light/dark cycles. Distance (mm) is represented over the 2 hours in 1-minute bins where n=36.

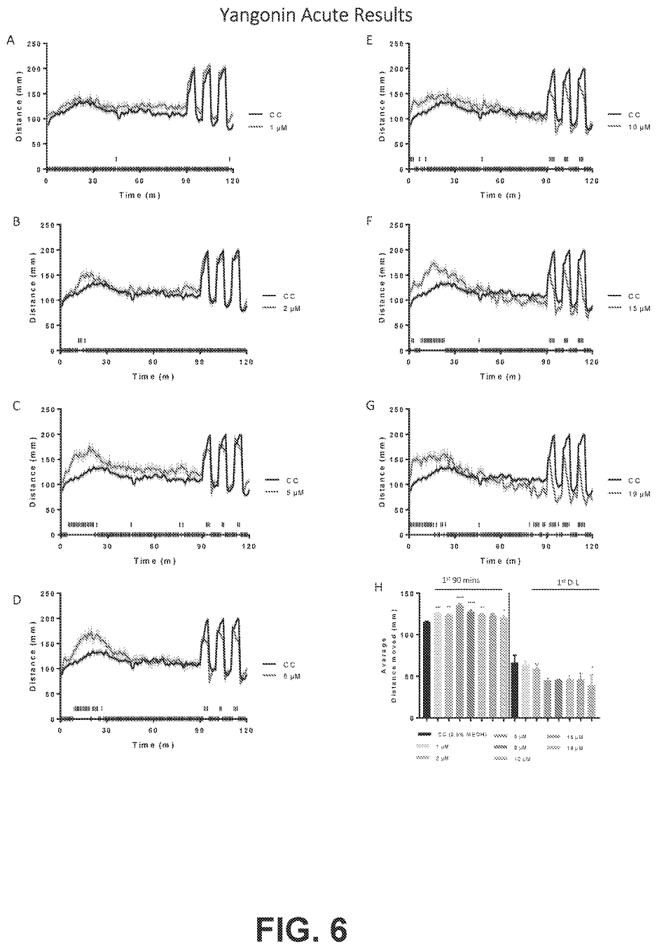

FIG. 6 shows (A-G) concentration response profile following acute exposure to increasing concentrations of Yangonin. Distance traveled±SEM is presented in millimeters and time in minutes. Each trace represents an n=36 (treatments) to 96 (control) larvae. Significance (dashed black line) versus controls was measured by 2-Way ANOVA followed by a Dunnett's multiple comparison test for each 60 second time window and displayed as a positive value on each graph where p<0.05. (H) Average distance moved (mm) in the first 90 minutes (light only) representing baseline larval activity and the larval response to the dark (average distance moved in the first 5 minute dark period-average activity preceding 5 minutes of dark). Significance versus controls was measured by 1-Way ANOVA followed by a Dunnett's multiple comparison test.

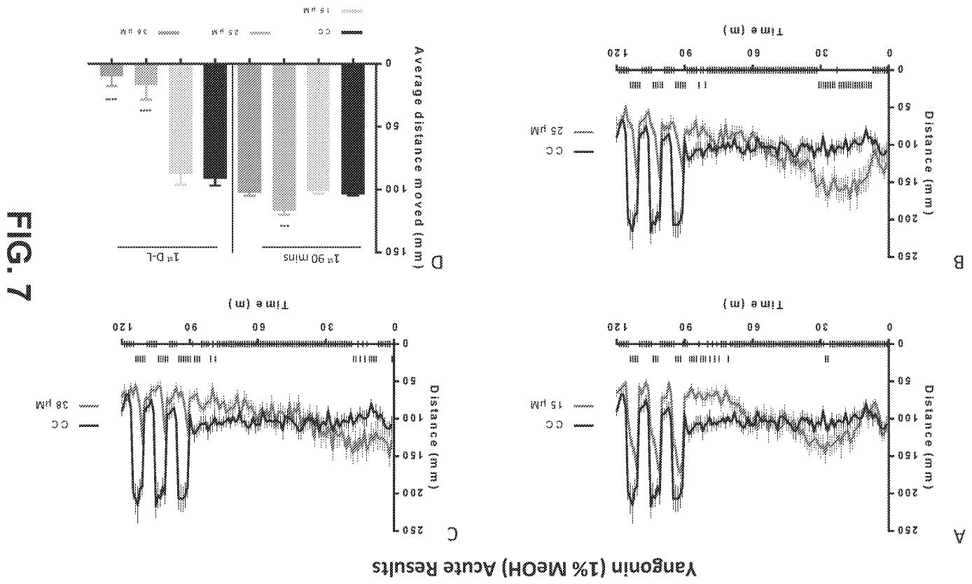

FIG. 7 shows (A-C) concentration response profile following acute exposure to increasing concentrations of Yangonin (1% MeOH). Distance traveled±SEM is presented in millimeters and time in minutes. Each trace represents an n=36 (treatments) to 96 (control) larvae. Significance (dashed black line) versus controls was measured by 2-Way ANOVA followed by a Dunnett's multiple comparison test for each 60 second time window and displayed as a positive value on each graph where p<0.05. (D) Average distance moved (mm) in the first 90 minutes (light only) representing baseline larval activity and the larval response to the dark (average distance moved in the first 5 minute dark period-average activity preceding 5 minutes of dark). Significance versus controls was measured by 1-Way ANOVA followed by a Dunnett's multiple comparison test.

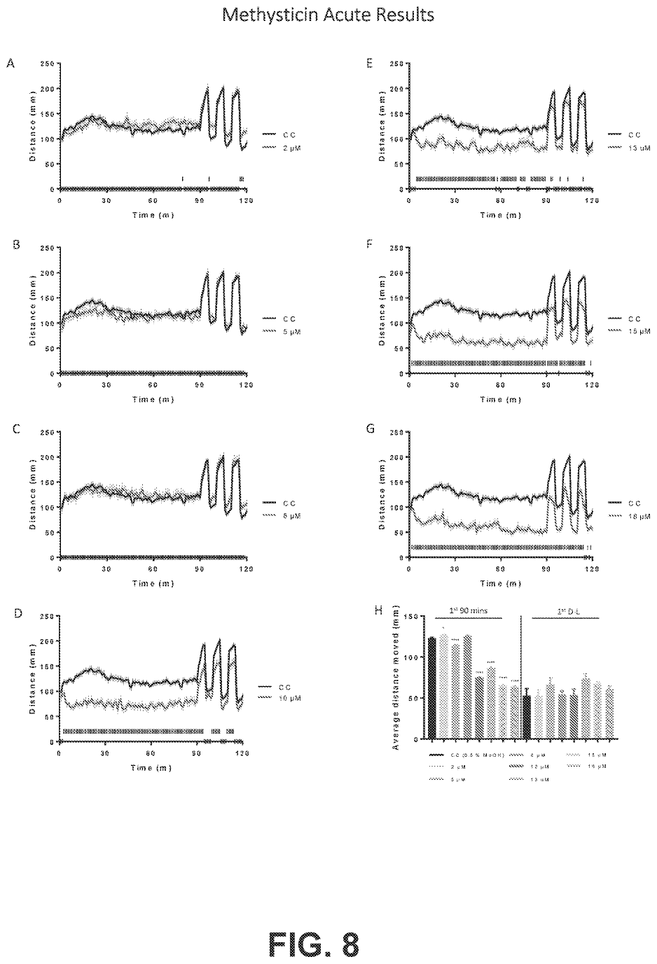

FIG. 8 shows (A-G) concentration response profile following acute exposure to increasing concentrations of Methysticin. Distance traveled±SEM is presented in millimeters and time in minutes. Each trace represents an n=36 (treatments) to 96 (control) larvae. Significance (dashed black line) versus controls was measured by 2-Way ANOVA followed by a Dunnett's multiple comparison test for each 60 second time window and displayed as a positive value on each graph where p<0.05. (H) Average distance moved (mm) in the first 90 minutes (light only) representing baseline larval activity and the larval response to the dark (average distance moved in the first 5 minute dark period-average activity preceding 5 minutes of dark). Significance versus controls was measured by 1-Way ANOVA followed by a Dunnett's multiple comparison test.

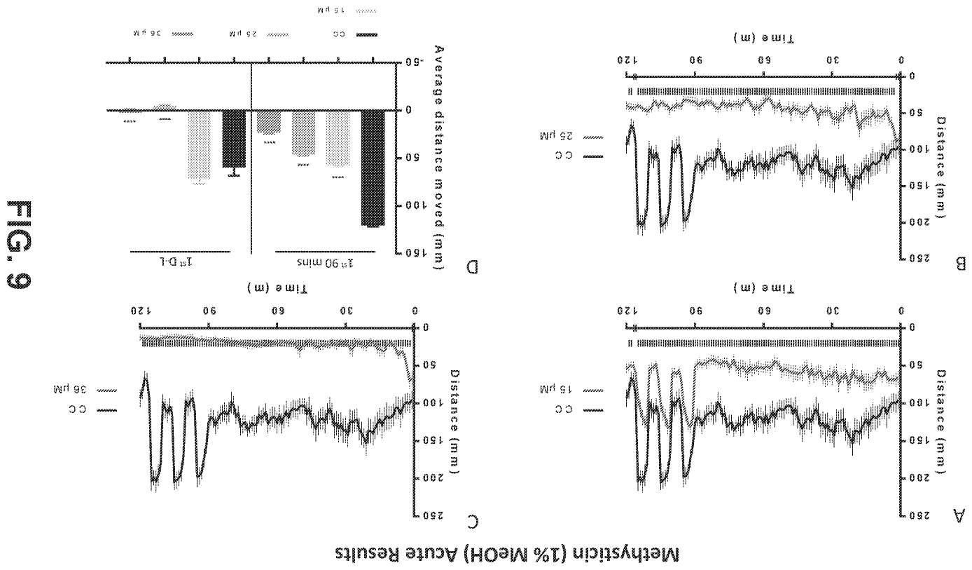

FIG. 9 shows (A-C) concentration response profile following acute exposure to increasing concentrations of Methysticin (1% MeOH). Distance traveled±SEM is presented in millimeters and time in minutes. Each trace represents an n=36 (treatments) to 96 (control) larvae. Significance (dashed black line) versus controls was measured by 2-Way ANOVA followed by a Dunnett's multiple comparison test for each 60 second time window and displayed as a positive value on each graph where p<0.05. (D) Average distance moved (mm) in the first 90 minutes (light only) representing baseline larval activity and the larval response to the dark (average distance moved in the first 5 minute dark period-average activity preceding 5 minutes of dark). Significance versus controls was measured by 1-Way ANOVA followed by a Dunnett's multiple comparison test.

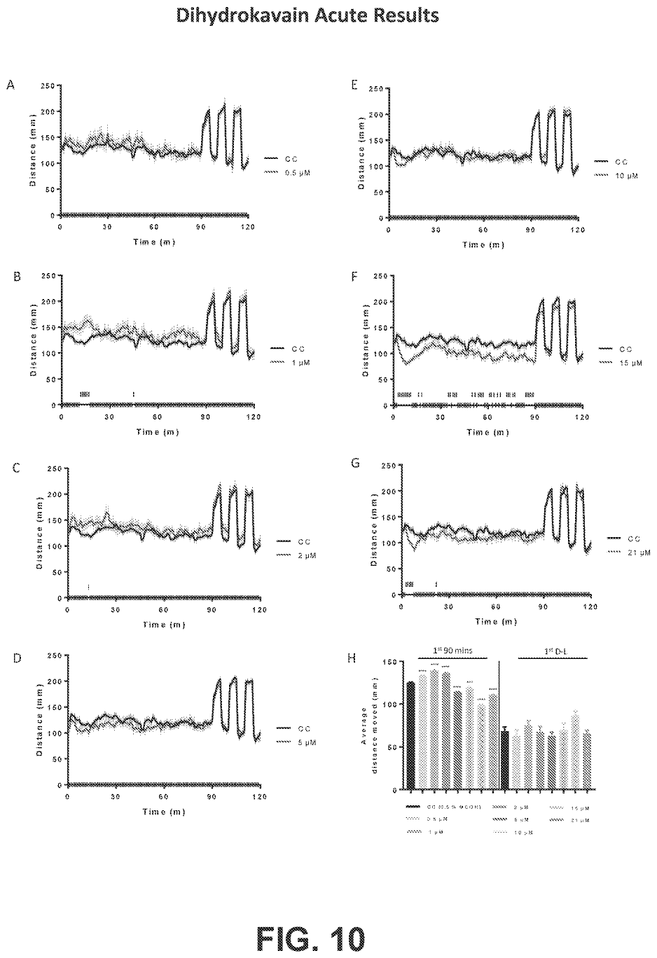

FIG. 10 shows (A-G) concentration response profile following acute exposure to increasing concentrations of Dihydrokavain. Distance traveled±SEM is presented in millimeters and time in minutes. Each trace represents an n=36 (treatments) to 36 (control) larvae. Significance (dashed black line) versus controls was measured by 2-Way ANOVA followed by a Dunnett's multiple comparison test for each 60 second time window and displayed as a positive value on each graph where p<0.05. (H) Average distance moved (mm) in the first 90 minutes (light only) representing baseline larval activity and the larval response to the dark (average distance moved in the first 5 minute dark period-average activity preceding 5 minutes of dark). Significance versus controls was measured by 1-Way ANOVA followed by a Dunnett's multiple comparison test.

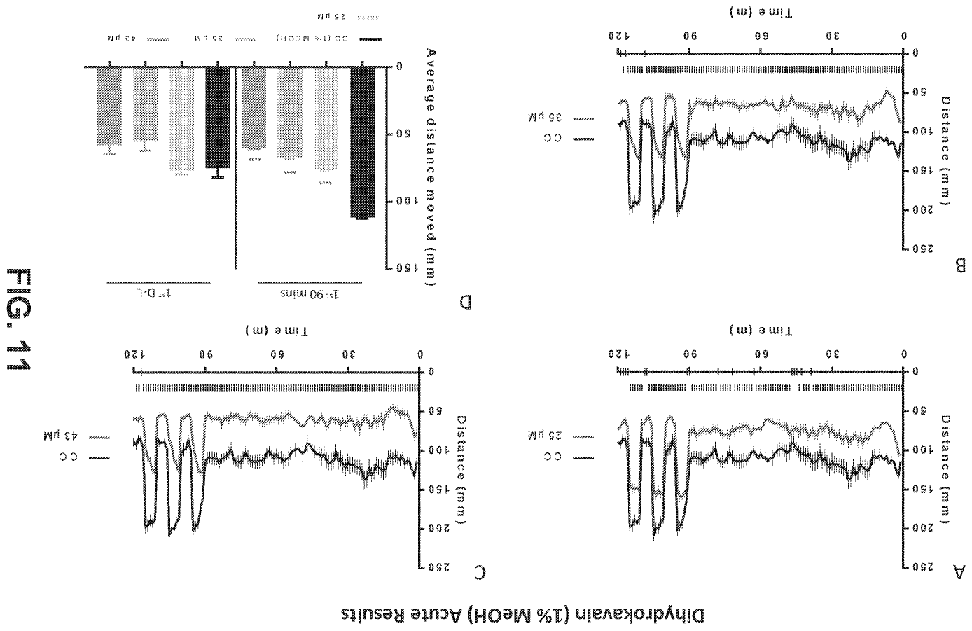

FIG. 11 shows (A-C) concentration response profile following acute exposure to increasing concentrations of Dihydrokavain (1% MeOH). Distance traveled±SEM is presented in millimeters and time in minutes. Each trace represents an n=36 (treatments) to 36 (control) larvae. Significance (dashed black line) versus controls was measured by 2-Way ANOVA followed by a Dunnett's multiple comparison test for each 60 second time window and displayed as a positive value on each graph where p<0.05. (D) Average distance moved (mm) in the first 90 minutes (light only) representing baseline larval activity and the larval response to the dark (average distance moved in the first 5 minute dark period-average activity preceding 5 minutes of dark). Significance versus controls was measured by 1-Way ANOVA followed by a Dunnett's multiple comparison test.

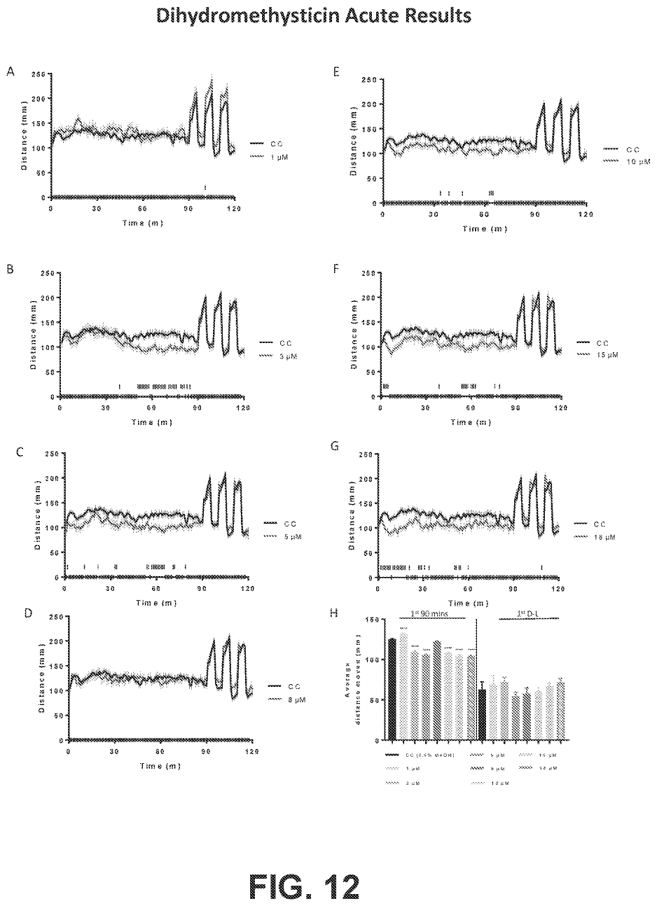

FIG. 12 shows (A-G) concentration response profile following acute exposure to increasing concentrations of Dihydromethysticin. Distance traveled±SEM is presented in millimeters and time in minutes. Each trace represents an n=36 (treatments) to 36 (control) larvae. Significance (dashed black line) versus controls was measured by 2-Way ANOVA followed by a Dunnett's multiple comparison test for each 60 second time window and displayed as a positive value on each graph where p<0.05. (H) Average distance moved (mm) in the first 90 minutes (light only) representing baseline larval activity and the larval response to the dark (average distance moved in the first 5 minute dark period-average activity preceding 5 minutes of dark). Significance versus controls was measured by 1-Way ANOVA followed by a Dunnett's multiple comparison test.

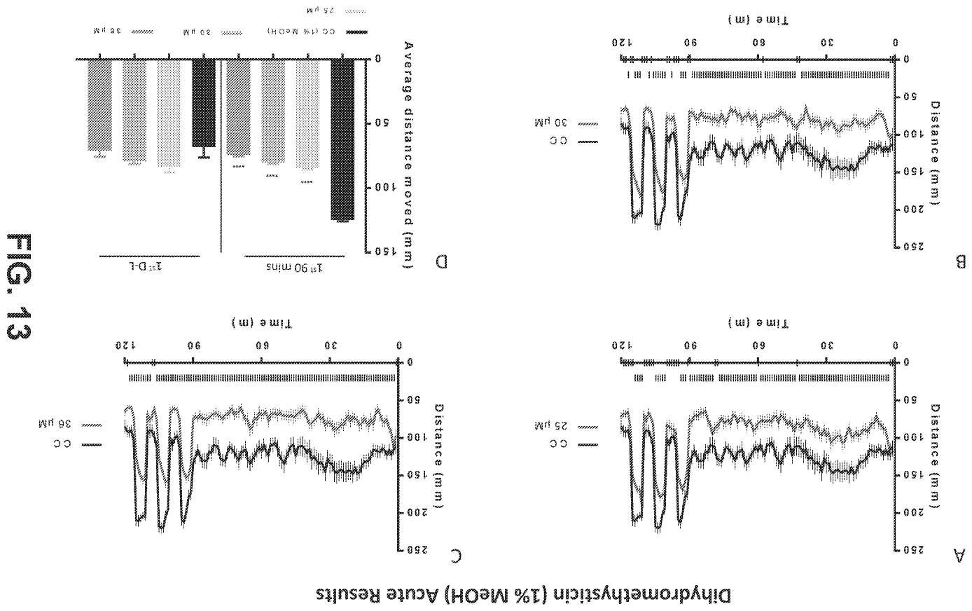

FIG. 13 shows (A-C) concentration response profile following acute exposure to increasing concentrations of Dihydromethysticin. Distance traveled±SEM is presented in millimeters and time in minutes. Each trace represents an n=36 (treatments) to 36 (control) larvae. Significance (dashed black line) versus controls was measured by 2-Way ANOVA followed by a Dunnett's multiple comparison test for each 60 second time window and displayed as a positive value on each graph where p<0.05. (D) Average distance moved (mm) in the first 90 minutes (light only) representing baseline larval activity and the larval response to the dark (average distance moved in the first 5 minute dark period-average activity preceding 5 minutes of dark). Significance versus controls was measured by 1-Way ANOVA followed by a Dunnett's multiple comparison test.

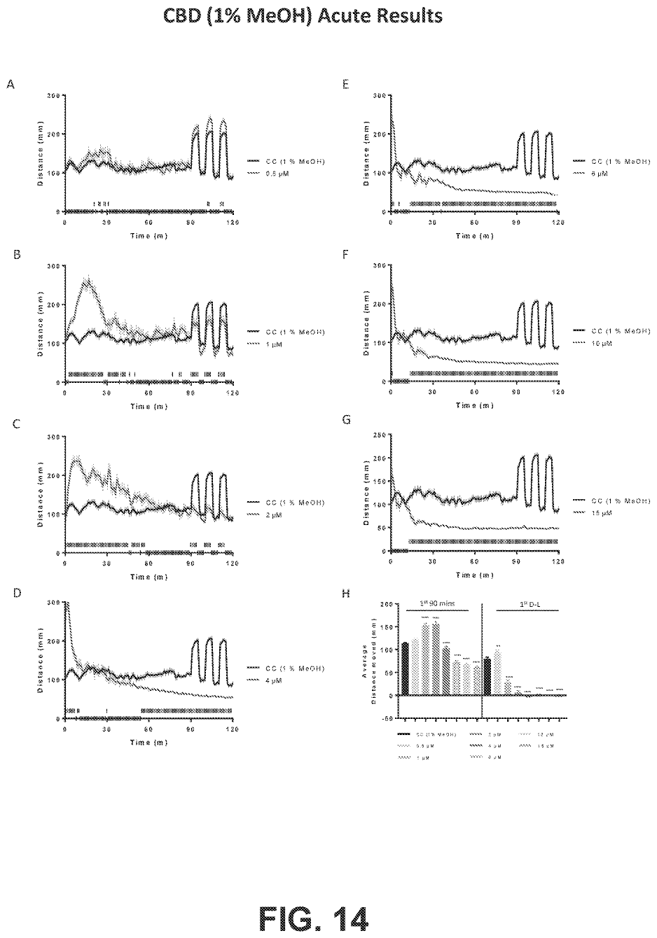

FIG. 14 shows (A-G) concentration response profile following acute exposure to increasing concentrations of CBD (1% MeOH). Distance traveled±SEM is presented in millimeters and time in minutes. Each trace represents an n=24 (treatments) to 96 (control) larvae. Significance (dashed black line) versus controls was measured by 2-Way ANOVA followed by a Dunnett's multiple comparison test for each 60 second time window and displayed as a positive value on each graph where p<0.05. (H) Average distance moved (mm) in the first 90 minutes (light only) representing baseline larval activity and the larval response to the dark (average distance moved in the first 5 minute dark period-average activity preceding 5 minutes of dark). Significance versus controls was measured by 1-Way ANOVA followed by a Dunnett's multiple comparison test.

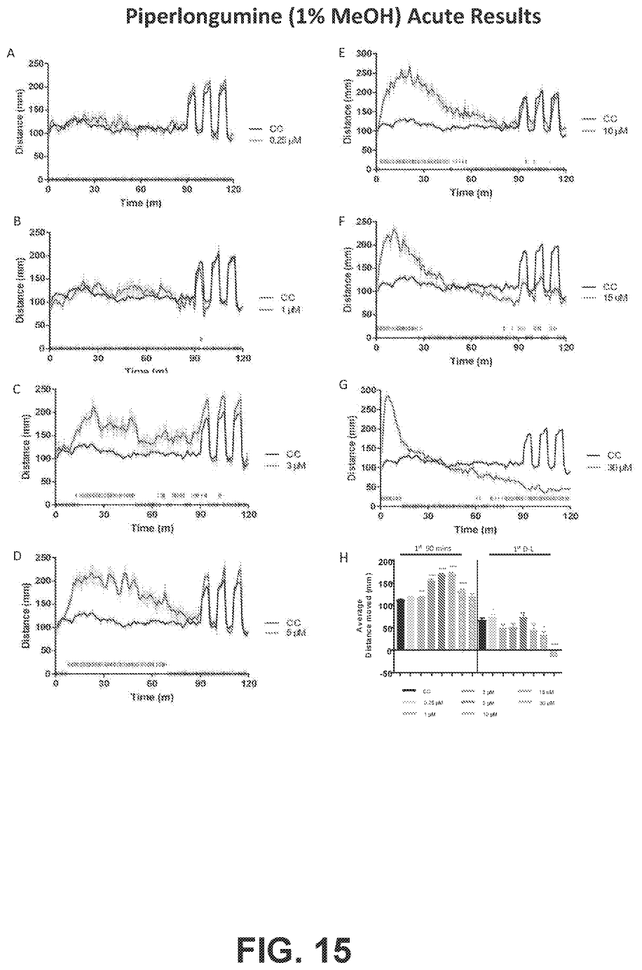

FIG. 15 shows (A-G) concentration response profile following acute exposure to increasing concentrations of Piperlongumine (1% MeOH). Distance traveled±SEM is presented in millimeters and time in minutes. Each trace represents an n=24 (treatments) to 96 (control) larvae. Significance (dashed black line) versus controls was measured by 2-Way ANOVA followed by a Dunnett's multiple comparison test for each 60 second time window and displayed as a positive value on each graph where p<0.05. (H) Average distance moved (mm) in the first 90 minutes (light only) representing baseline larval activity and the larval response to the dark (average distance moved in the first 5 minute dark period-average activity preceding 5 minutes of dark). Significance versus controls was measured by 1-Way ANOVA followed by a Dunnett's multiple comparison test.

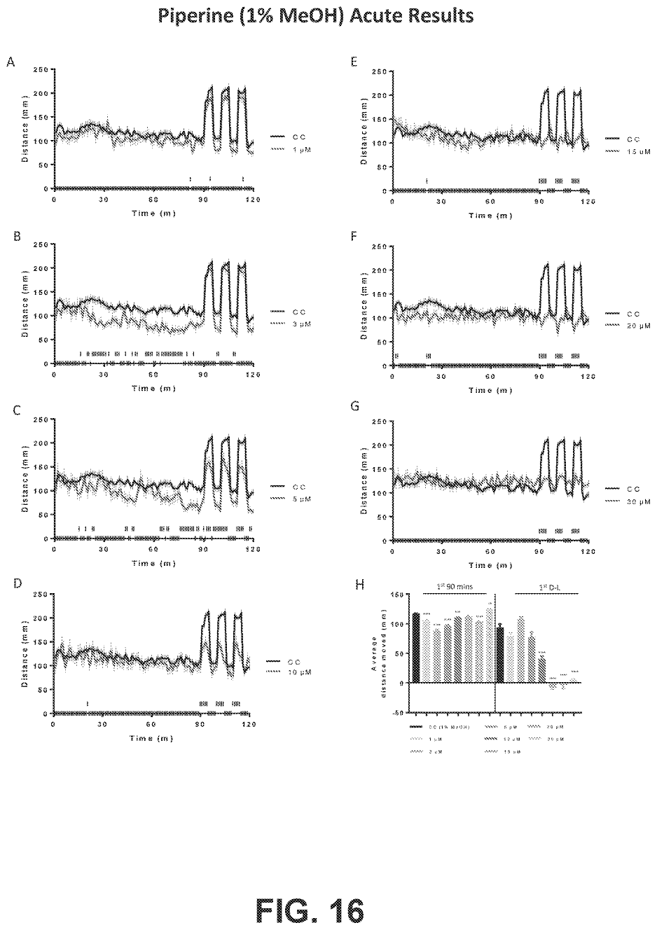

FIG. 16 shows (A-G) concentration response profile following acute exposure to increasing concentrations of Piperine (1% MeOH). Distance traveled±SEM is presented in millimeters and time in minutes. Each trace represents an n=24 (treatments) to 60 (control) larvae. Significance (dashed black line) versus controls was measured by 2-Way ANOVA followed by a Dunnett's multiple comparison test for each 60 second time window and displayed as a positive value on each graph where p<0.05. (H) Average distance moved (mm) in the first 90 minutes (light only) representing baseline larval activity and the larval response to the dark (average distance moved in the first 5 minute dark period-average activity preceding 5 minutes of dark). Significance versus controls was measured by 1-Way ANOVA followed by a Dunnett's multiple comparison test.

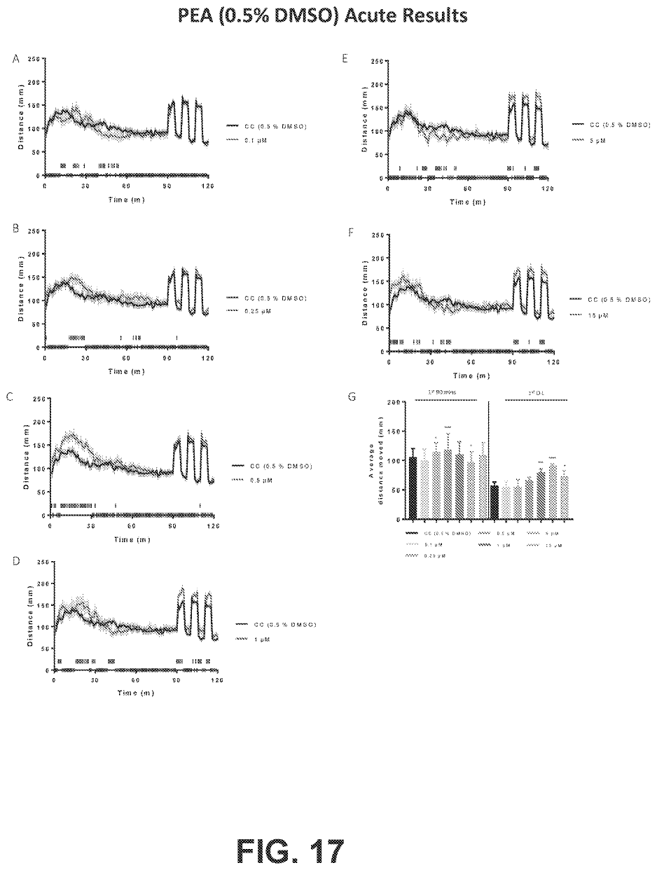

FIG. 17 shows (A-F) concentration response profile following acute exposure to increasing concentrations of PEA (0.5% DMSO). Distance traveled±SEM is presented in millimeters and time in minutes. Each trace represents an n=24 (treatments) to 48 (control) larvae. Significance (dashed black line) versus controls was measured by 2-Way ANOVA followed by a Dunnett's multiple comparison test for each 60 second time window and displayed as a positive value on each graph where p<0.05. (G) Average distance moved (mm) in the first 90 minutes (light only) representing baseline larval activity and the larval response to the dark (average distance moved in the first 5 minute dark period-average activity preceding 5 minutes of dark). Significance versus controls was measured by 1-Way ANOVA followed by a Dunnett's multiple comparison test.

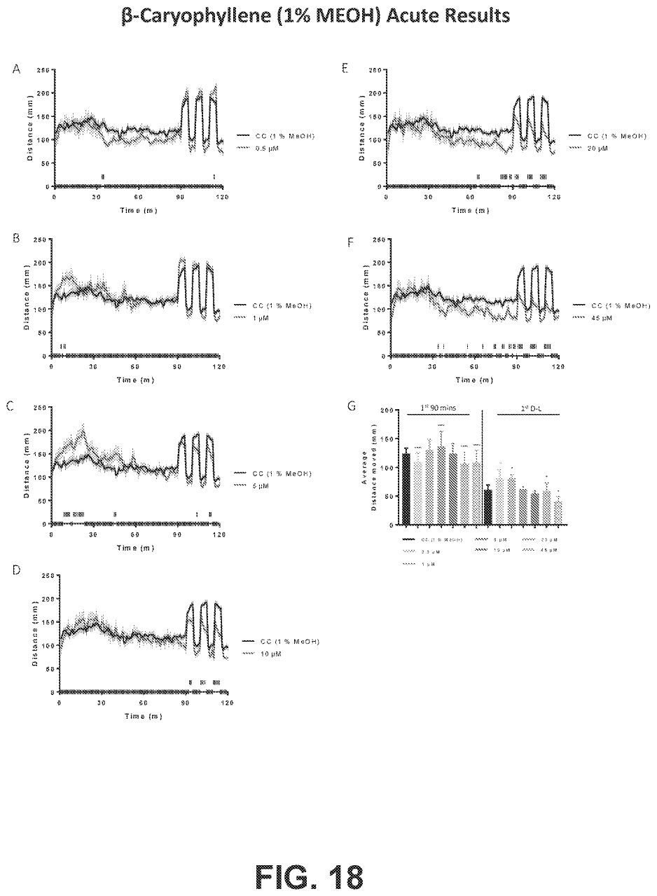

FIG. 18 shows (A-F) concentration response profile following acute exposure to increasing concentrations of B-Caryophyllene (0.5% DMSO). Distance traveled±SEM is presented in millimeters and time in minutes. Each trace represents an n=24 (treatments) to 48 (control) larvae. Significance (dashed black line) versus controls was measured by 2-Way ANOVA followed by a Dunnett's multiple comparison test for each 60 second time window and displayed as a positive value on each graph where p<0.05. (G) Average distance moved (mm) in the first 90 minutes (light only) representing baseline larval activity and the larval response to the dark (average distance moved in the first 5 minute dark period-average activity preceding 5 minutes of dark). Significance versus controls was measured by 1-Way ANOVA followed by a Dunnett's multiple comparison test.

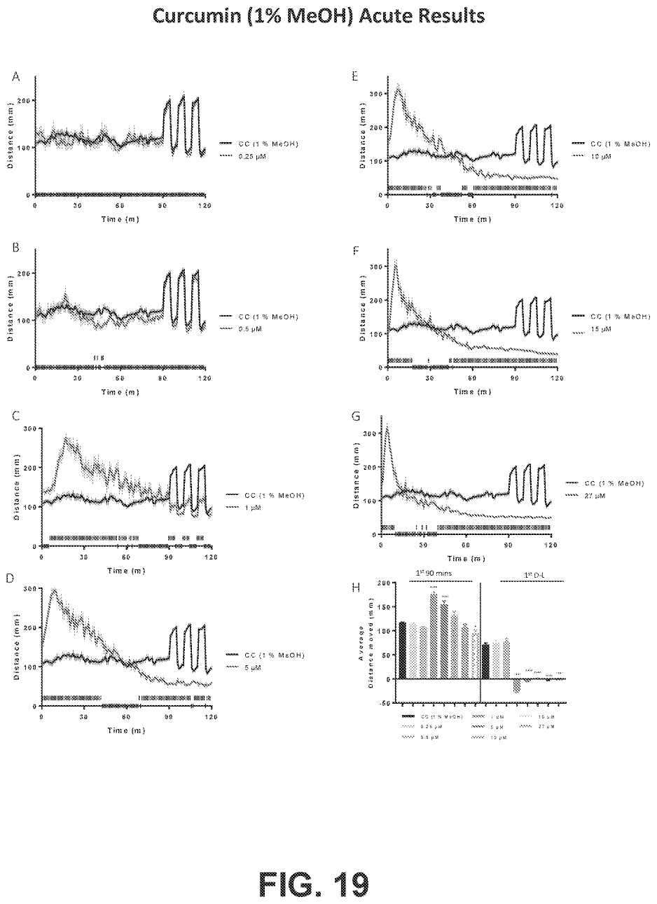

FIG. 19 shows (A-G) concentration response profile following acute exposure to increasing concentrations of Curcumin (1% MeOH). Distance traveled±SEM is presented in millimeters and time in minutes. Each trace represents an n=24 (treatments) to 48 (control) larvae. Significance (dashed black line) versus controls was measured by 2-Way ANOVA followed by a Dunnett's multiple comparison test for each 60 second time window and displayed as a positive value on each graph where p<0.05. (H) Average distance moved (mm) in the first 90 minutes (light only) representing baseline larval activity and the larval response to the dark (average distance moved in the first 5 minute dark period-average activity preceding 5 minutes of dark). Significance versus controls was measured by 1-Way ANOVA followed by a Dunnett's multiple comparison test.

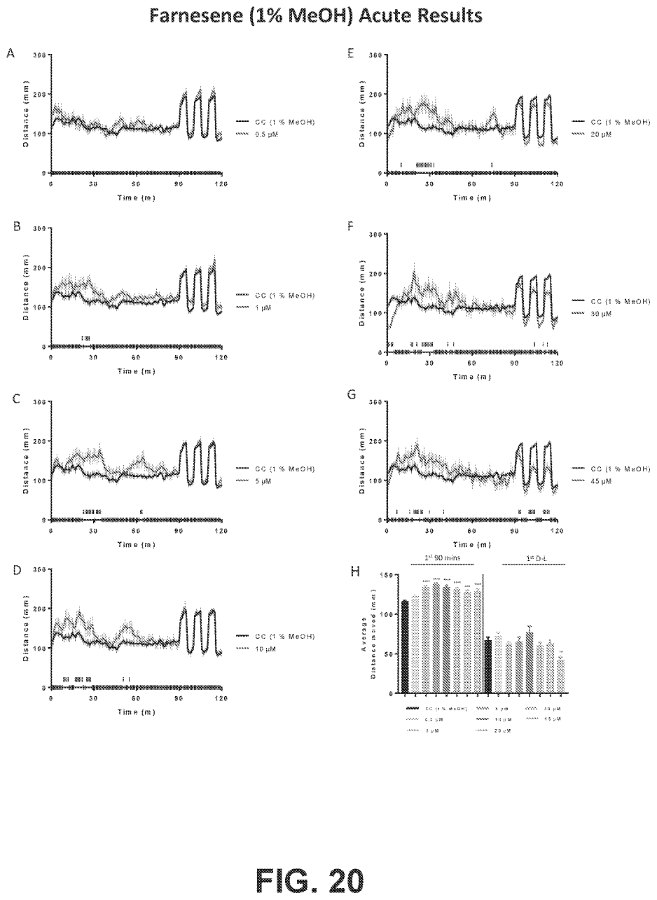

FIG. 20 shows (A-G) concentration response profile following acute exposure to increasing concentrations of Farnesene (1% MeOH). Distance traveled±SEM is presented in millimeters and time in minutes. Each trace represents an n=24 (treatments) to 72 (control) larvae. Significance (dashed black line) versus controls was measured by 2-Way ANOVA followed by a Dunnett's multiple comparison test for each 60 second time window and displayed as a positive value on each graph where p<0.05. (H) Average distance moved (mm) in the first 90 minutes (light only) representing baseline larval activity and the larval response to the dark (average distance moved in the first 5 minute dark period-average activity preceding 5 minutes of dark). Significance versus controls was measured by 1-Way ANOVA followed by a Dunnett's multiple comparison test.

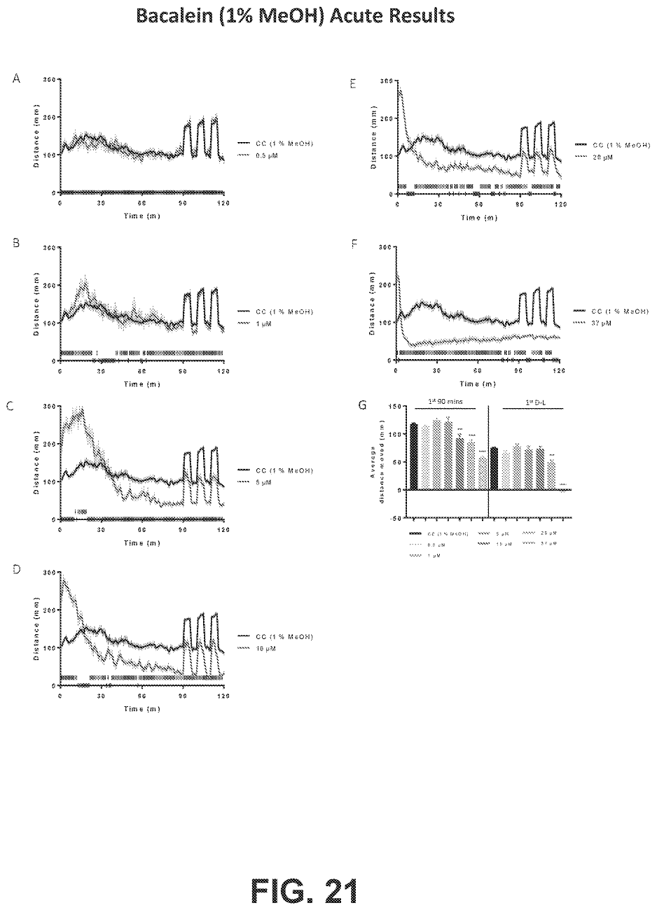

FIG. 21 shows (A-F) concentration response profile following acute exposure to Bacalein (1% MeOH). Distance traveled±SEM is presented in millimeters and time in minutes. Each trace represents an n=24 (treatments) to 48 (control) larvae. Significance (dashed black line) versus controls was measured by 2-Way ANOVA followed by a Dunnett's multiple comparison test for each 60 second time window and displayed as a positive value on each graph where p<0.05. (G) Average distance moved (mm) in the first 90 minutes (light only) representing baseline larval activity and the larval response to the dark (average distance moved in the first 5 minute dark period-average activity preceding 5 minutes of dark). Significance versus controls was measured by 1-Way ANOVA followed by a Dunnett's multiple comparison test.

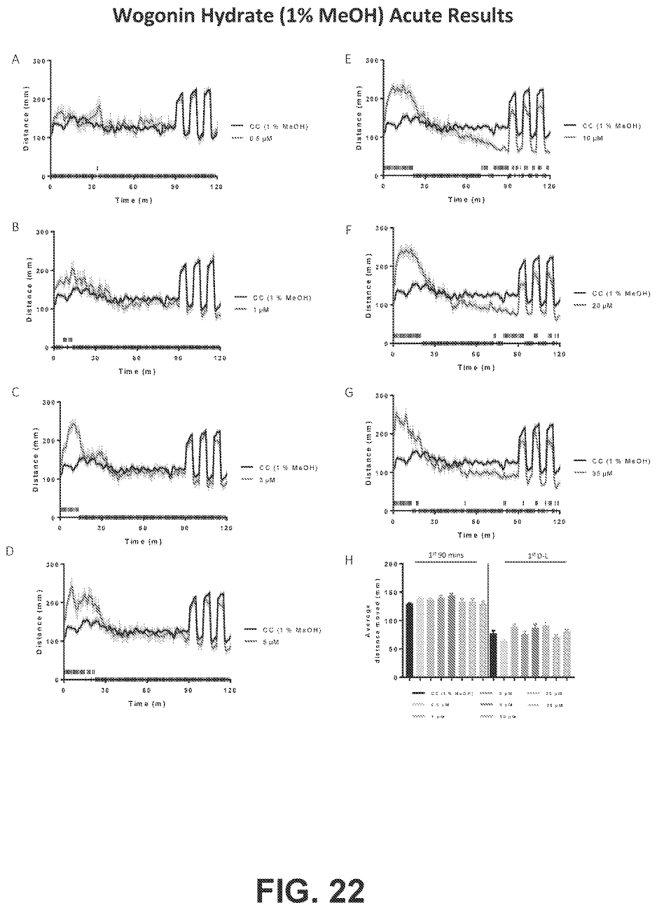

FIG. 22 shows (A-G) concentration response profile following acute exposure to increasing concentrations of Wogonin Hydrate (1% MeOH). Distance traveled±SEM is presented in millimeters and time in minutes. Each trace represents an n=24 (treatments) to 72 (control) larvae. Significance (dashed black line) versus controls was measured by 2-Way ANOVA followed by a Dunnett's multiple comparison test for each 60 second time window and displayed as a positive value on each graph where p<0.05. (H) Average distance moved (mm) in the first 90 minutes (light only) representing baseline larval activity and the larval response to the dark (average distance moved in the first 5 minute dark period-average activity preceding 5 minutes of dark). Significance versus controls was measured by 1-Way ANOVA followed by a Dunnett's multiple comparison test.

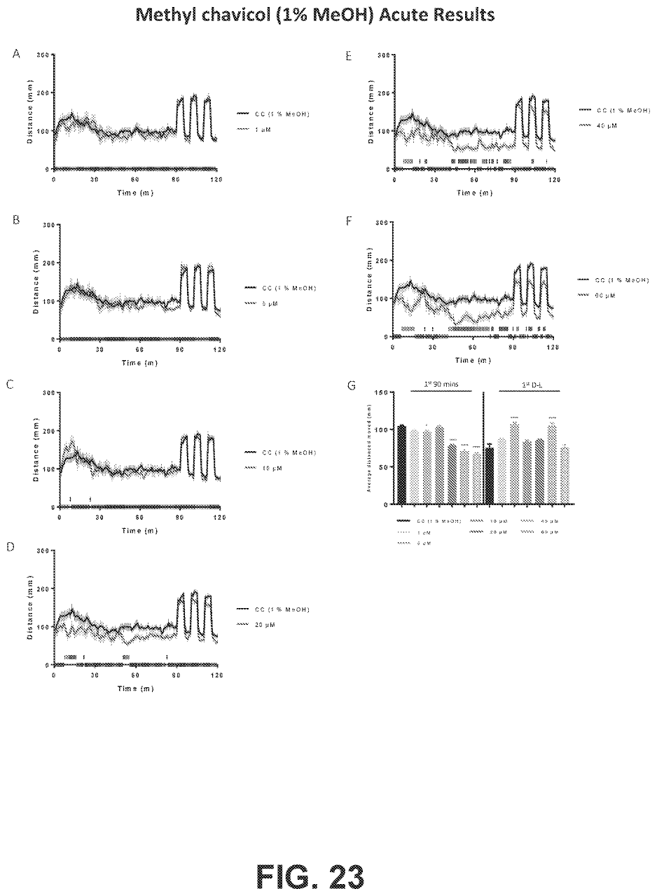

FIG. 23 shows (A-F) concentration response profile following acute exposure to increasing concentrations of Methyl chavicol (1% MeOH). Distance traveled±SEM is presented in millimeters and time in minutes. Each trace represents an n=24 (treatments) to 48 (control) larvae. Significance (dashed black line) versus controls was measured by 2-Way ANOVA followed by a Dunnett's multiple comparison test for each 60 second time window and displayed as a positive value on each graph where p<0.05. (G) Average distance moved (mm) in the first 90 minutes (light only) representing baseline larval activity and the larval response to the dark (average distance moved in the first 5 minute dark period-average activity preceding 5 minutes of dark). Significance versus controls was measured by 1-Way ANOVA followed by a Dunnett's multiple comparison test.

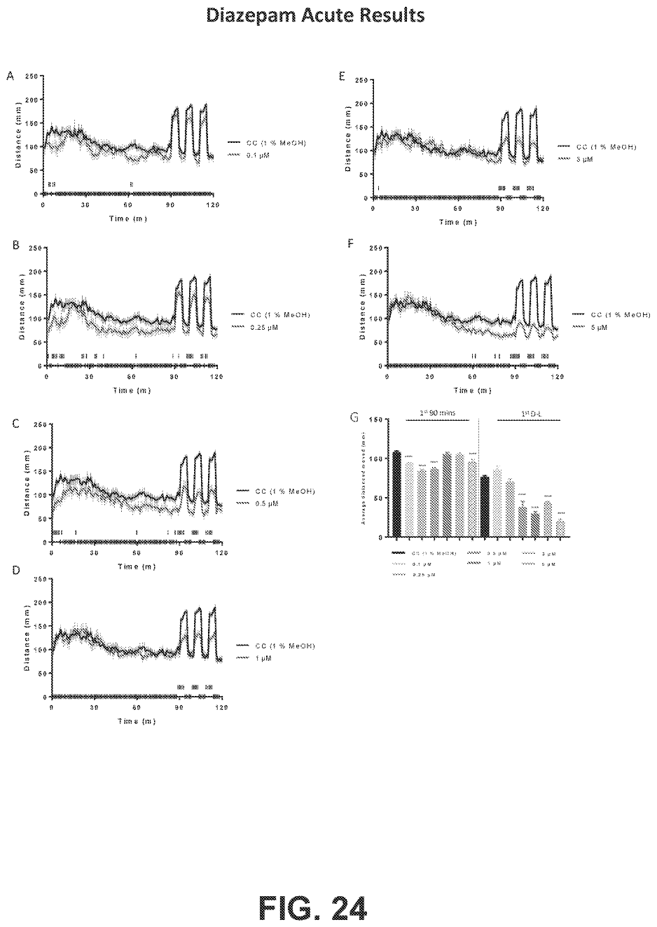

FIG. 24 shows (A-F) concentration response profile following acute exposure to increasing concentrations of Diazepam. Distance traveled±SEM is presented in millimeters and time in minutes. Each trace represents an n=36 (treatments) to 36 (control) larvae. Significance (dashed black line) versus controls was measured by 2-Way ANOVA followed by a Dunnett's multiple comparison test for each 60 second time window and displayed as a positive value on each graph where p<0.05. (G) Average distance moved (mm) in the first 90 minutes (light only) representing baseline larval activity and the larval response to the dark (average distance moved in the first 5 minute dark period-average activity preceding 5 minutes of dark). Significance versus controls was measured by 1-Way ANOVA followed by a Dunnett's multiple comparison test.

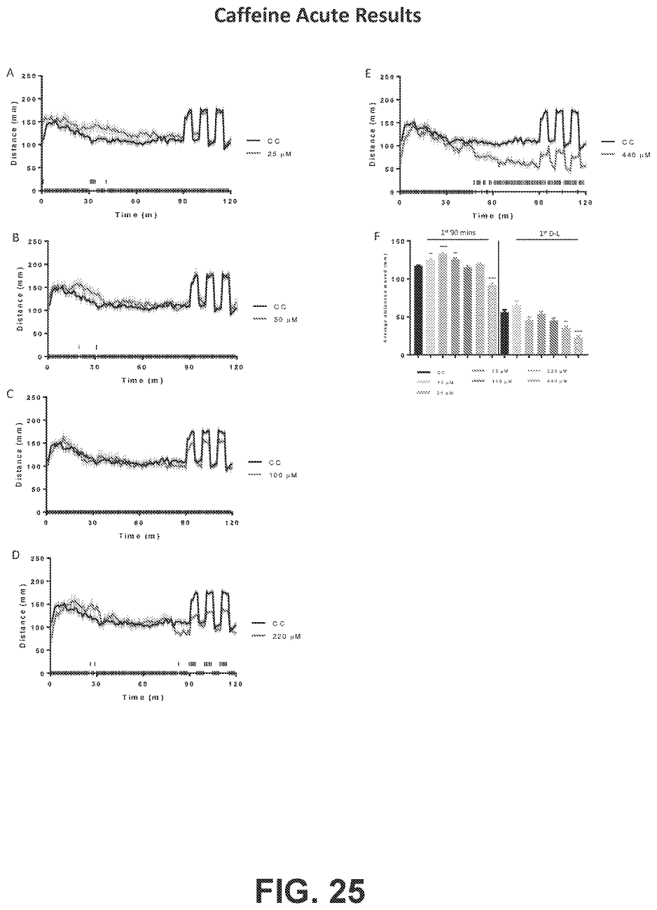

FIG. 25 shows (A-E) concentration response profile following acute exposure to increasing concentrations of Caffeine. Distance traveled±SEM is presented in millimeters and time in minutes. Each trace represents an n=36 (treatments) to 36 (control) larvae. Significance (dashed black line) versus controls was measured by 2-Way ANOVA followed by a Dunnett's multiple comparison test for each 60 second time window and displayed as a positive value on each graph where p<0.05. (F) Average distance moved (mm) in the first 90 minutes (light only) representing baseline larval activity and the larval response to the dark (average distance moved in the first 5 minute dark period-average activity preceding 5 minutes of dark). Significance versus controls was measured by 1-Way ANOVA followed by a Dunnett's multiple comparison test.

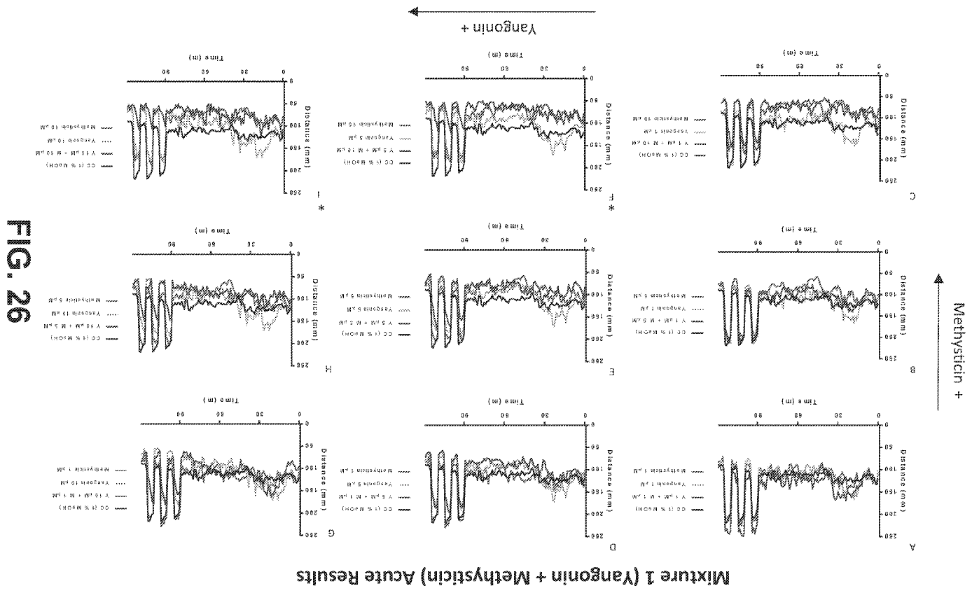

FIG. 26 shows concentration response profile following acute exposure to Mixture 1, concentration matched pure Yangonin/Methysticin and control treated larvae (1% MeOH). Distance traveled is presented in millimeters and time in minutes. Control n=32, treated n=16.

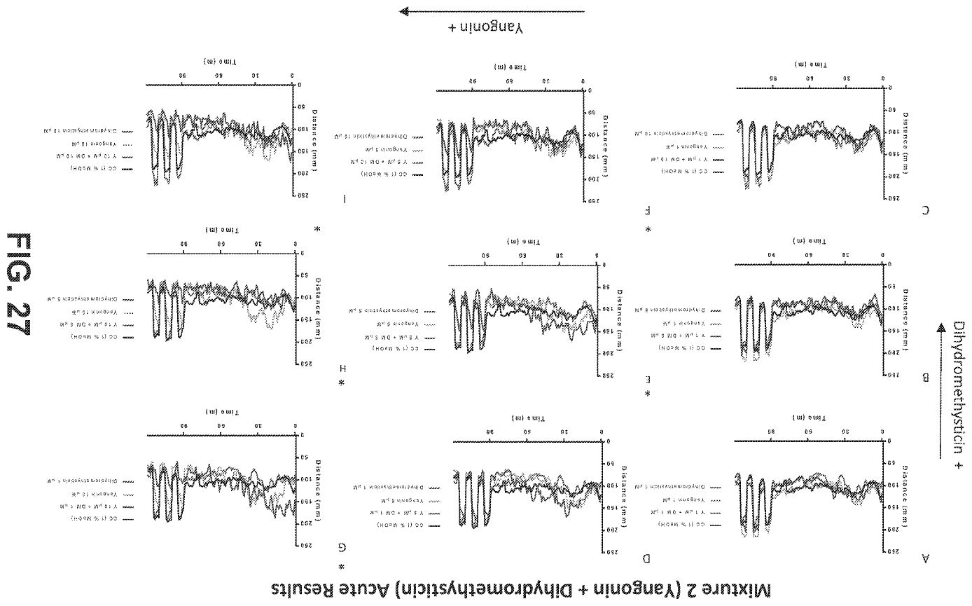

FIG. 27 shows concentration response profile following acute exposure to Mixture 2, concentration matched pure Yangonin/Dihydromethysticin and control treated larvae. (1% MeOH). Distance traveled is presented in millimeters and time in minutes. Each trace represents an n=24.

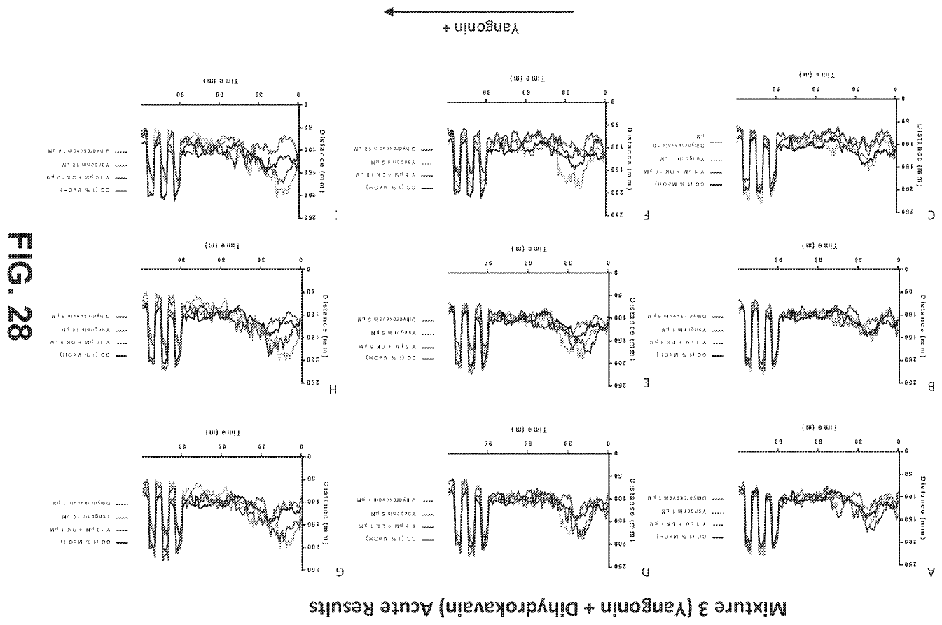

FIG. 28 shows concentration response profile following acute exposure to Mixture 3, concentration matched pure Yangonin/Dihydrokavain and control treated larvae (1% MeOH). Distance traveled is presented in millimeters and time in minutes. Control n=32, treated n=16.

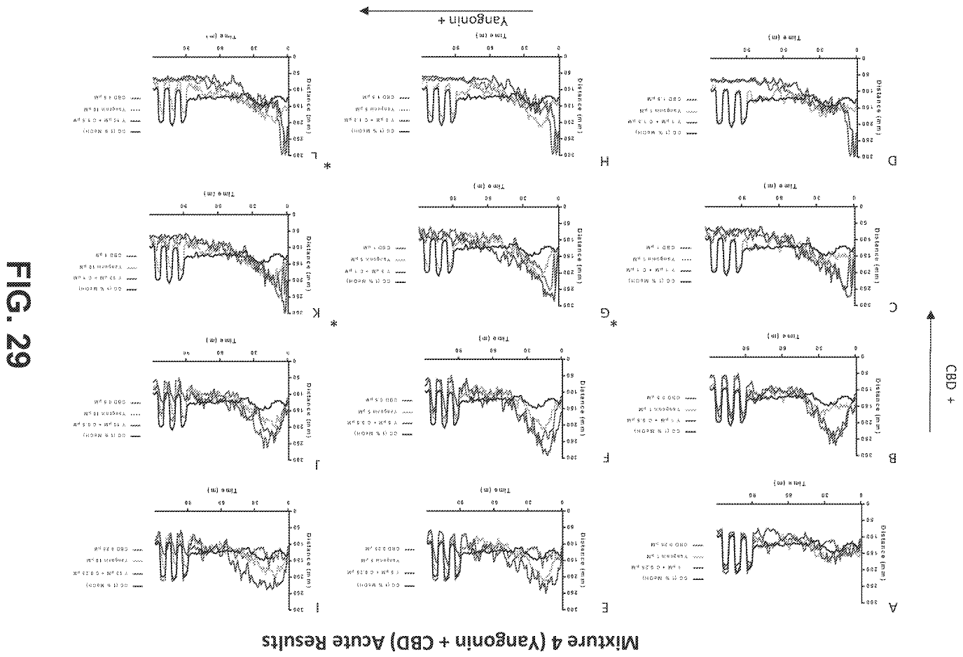

FIG. 29 shows concentration response profile following acute exposure to Mixture 4, concentration matched pure Yangonin/CBD and control treated larvae (1% MeOH). Distance traveled is presented in millimeters and time in minutes. Control n=32, treated n=16.

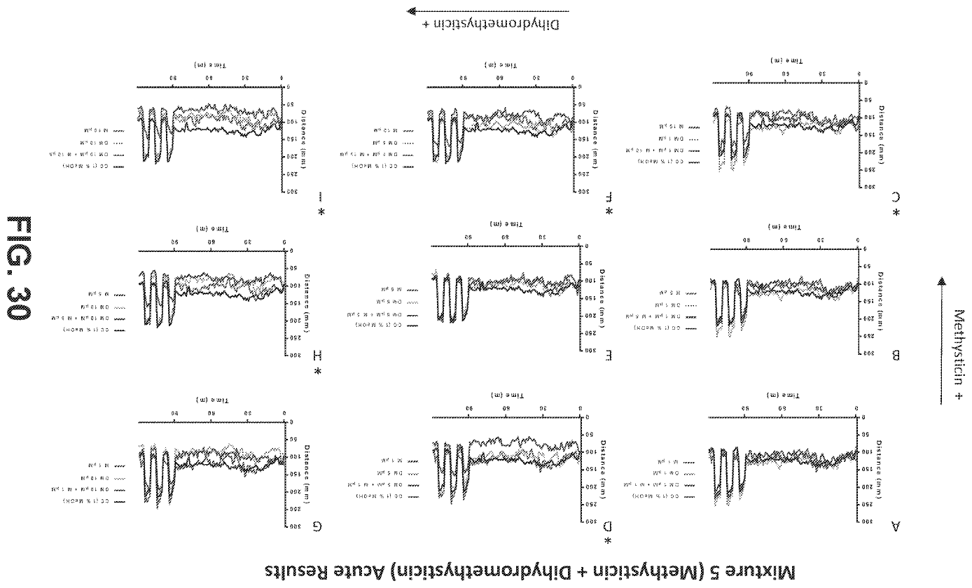

FIG. 30 shows concentration response profile following acute exposure to Mixture 5, concentration matched pure Methysticin/Dihydromethysticin and control treated larvae (1% MeOH). Distance traveled is presented in millimeters and time in minutes. Control n=32, treated n=16.

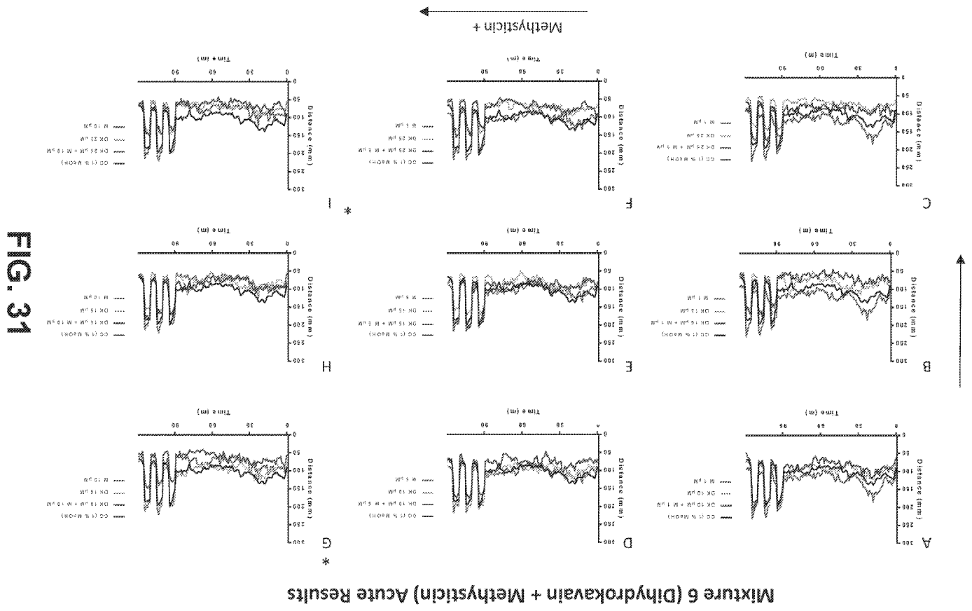

FIG. 31 shows concentration response profile following acute exposure to Mixture 6, concentration matched pure Dihydrokavain/Methysticin and control treated larvae (1% MeOH). Distance traveled is presented in millimeters and time in minutes. Control n=32, treated n=16.

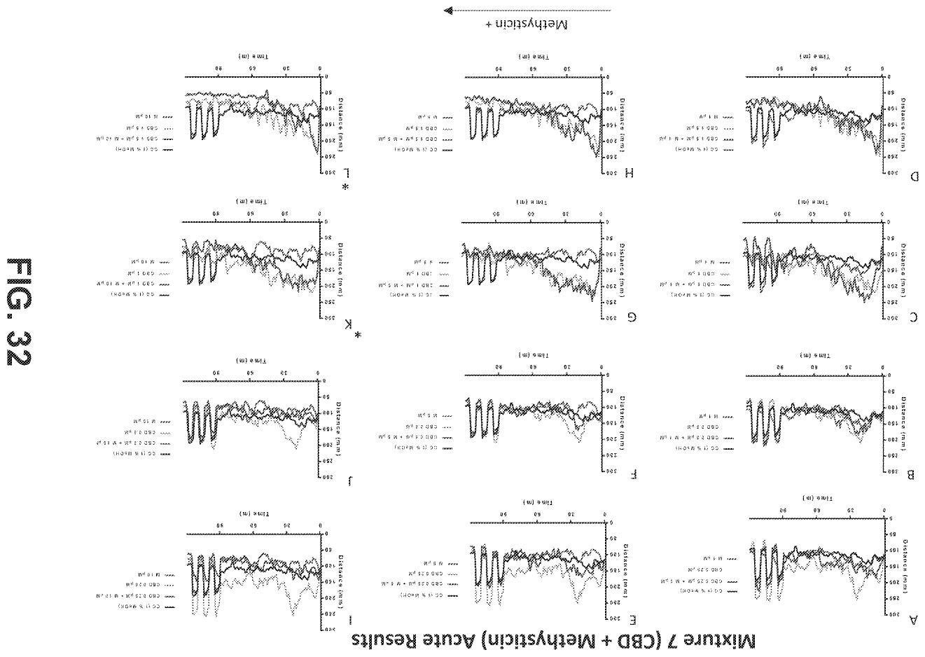

FIG. 32 shows concentration response profile following acute exposure to Mixture 7, concentration matched pure CBD/Methysticin and control treated larvae (1% MeOH). Distance traveled is presented in millimeters and time in minutes. Each trace represents an n=24.

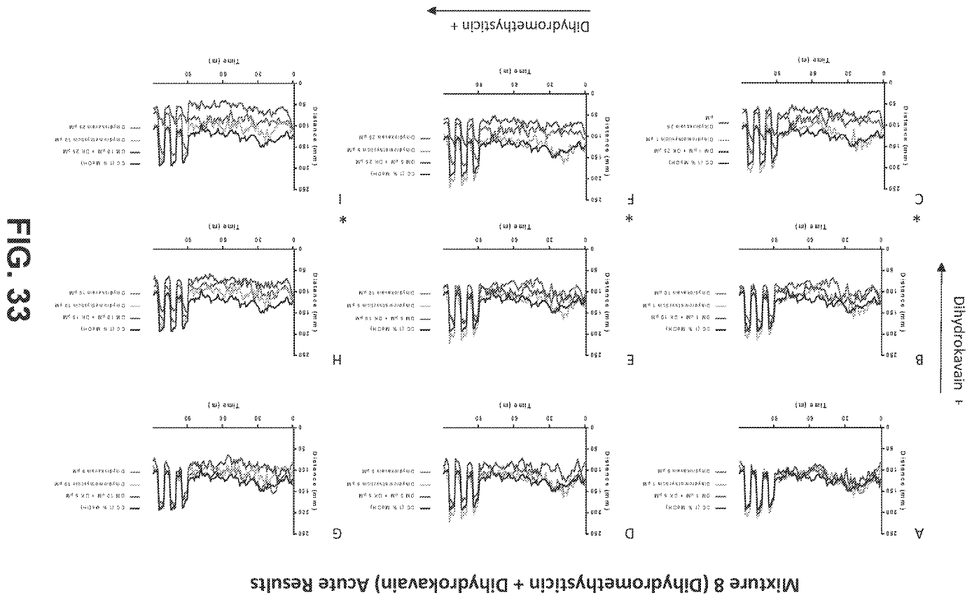

FIG. 33 shows concentration response profile following acute exposure to Mixture 8, concentration matched pure Dihydromethysticin/Dihydrokavain and control treated larvae (1% MeOH). Distance traveled is presented in millimeters and time in minutes. Control n=32, treated n=16.

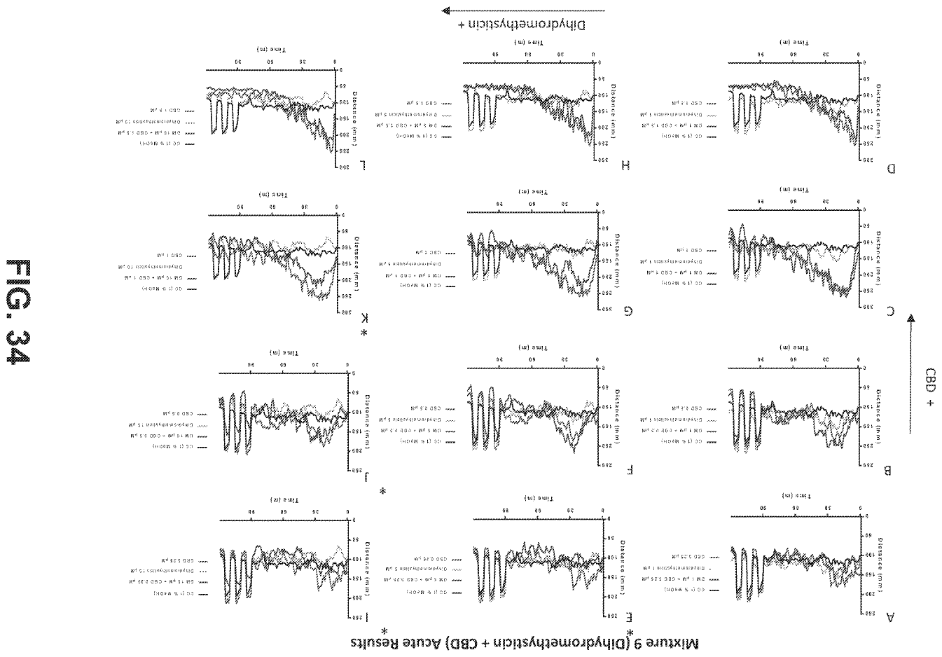

FIG. 34 shows concentration response profile following acute exposure to Mixture 9, concentration matched pure Dihydromethysticin/CBD and control treated larvae (1% MeOH). Distance traveled is presented in millimeters and time in minutes. Control n=32, treated n=16.

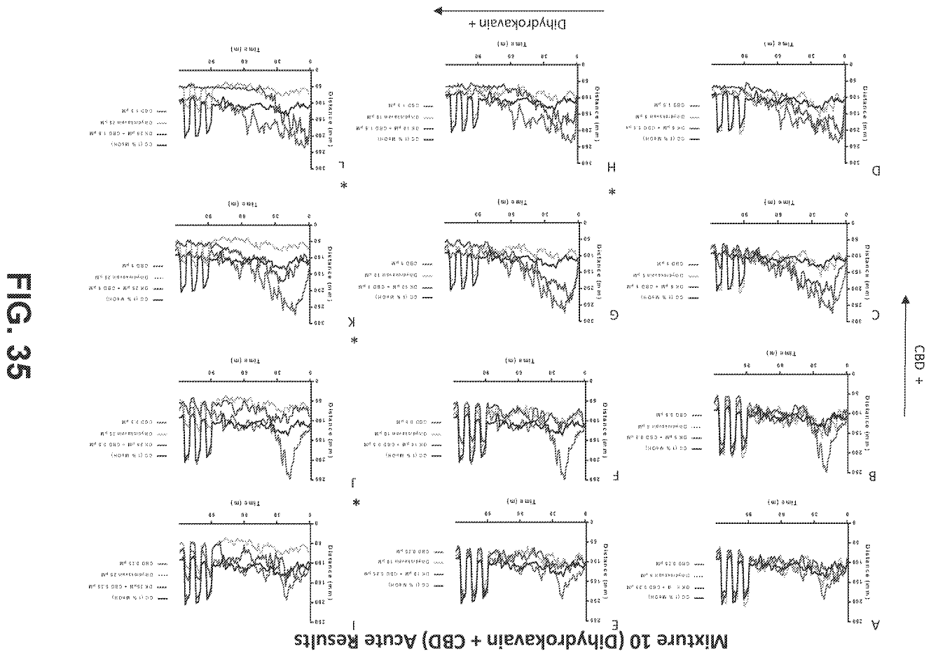

FIG. 35 shows concentration response profile following acute exposure to Mixture 10, concentration matched pure Dihydrokavain/CBD and control treated larvae (1% MeOH). Distance traveled is presented in millimeters and time in minutes. Control n=32, treated n=8-16.

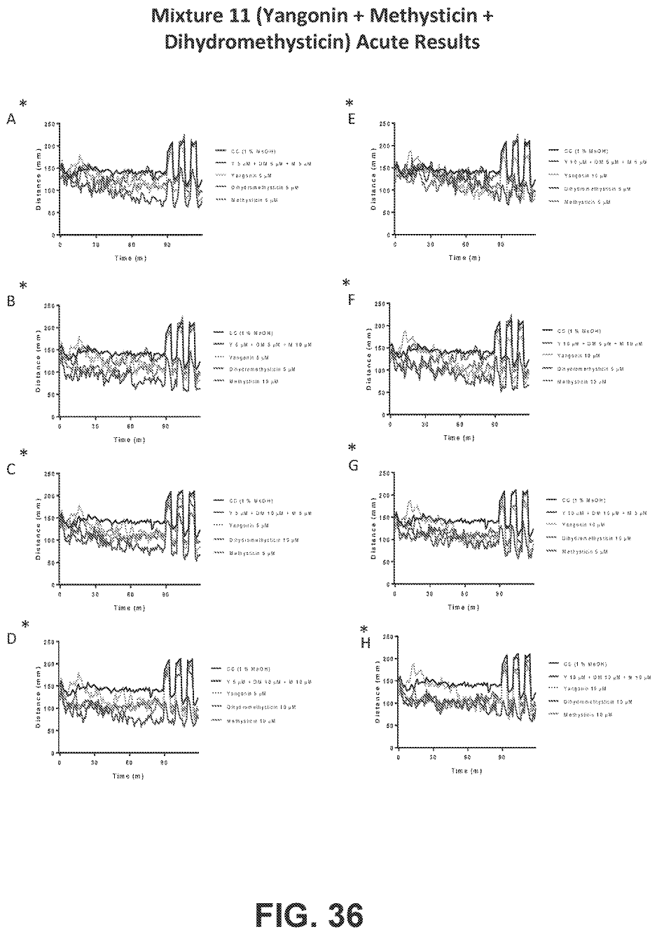

FIG. 36 shows concentration response profile following acute exposure to multiple concentrations of Mixture 11 (Yangonin, Dihydromethysticin and Methysticin) and control treated larvae (1% MeOH). Distance traveled is presented in millimeters and time in minutes. Control n=32, treated n=16.

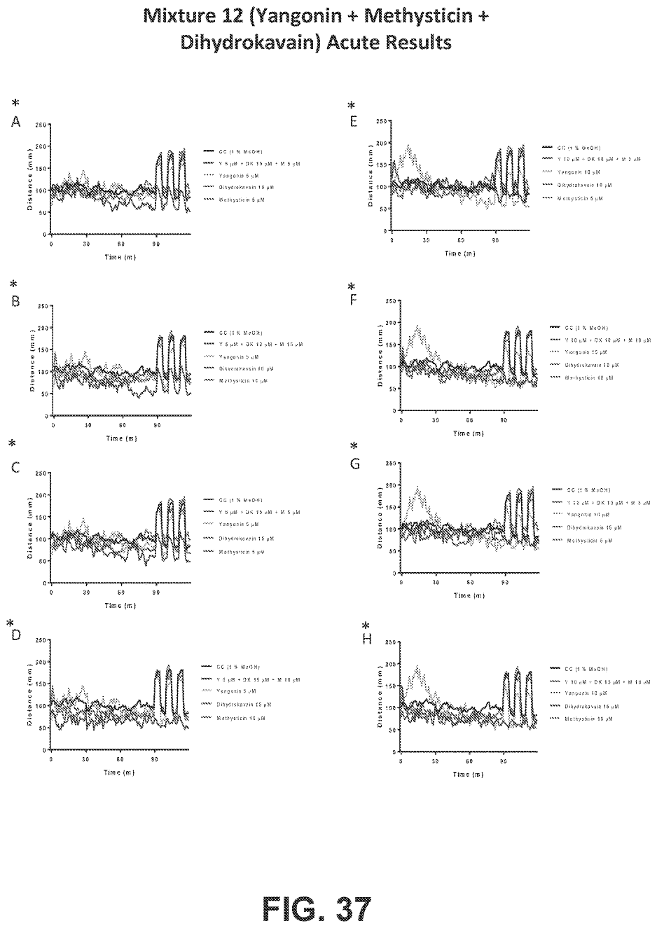

FIG. 37 shows concentration response profile following acute exposure to multiple concentrations of Mixture 12 (Yangonin, Dihydrokavain and Methysticin) and control treated larvae (1% MeOH). Distance traveled is presented in millimeters and time in minutes. Control n=32, treated n=16.

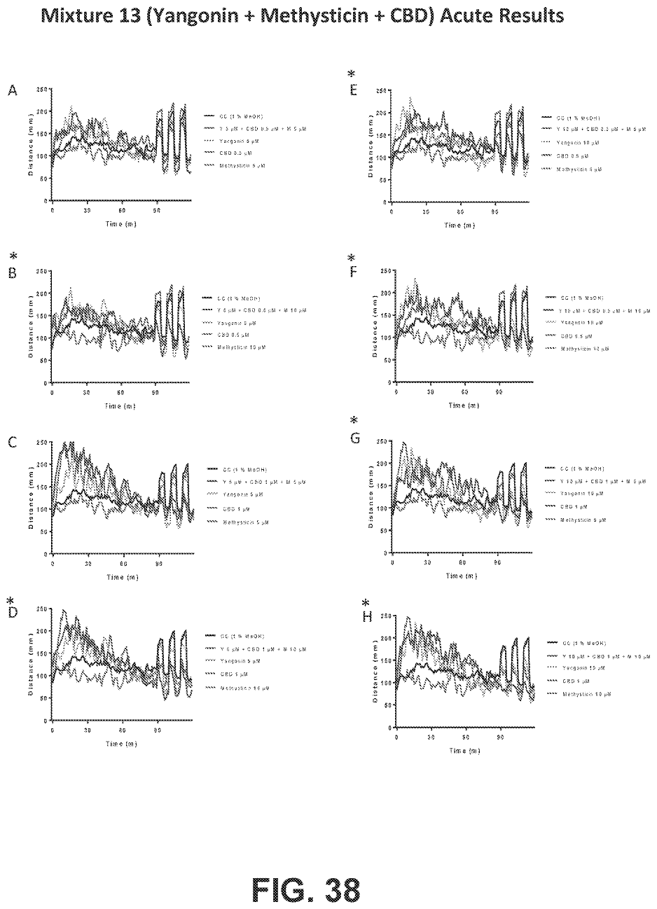

FIG. 38 shows concentration response profile following acute exposure to multiple concentrations of Mixture 13 (Yangonin, Methysticin and CBD) and control treated larvae (1% MeOH). Distance traveled is presented in millimeters and time in minutes. Control n=32, treated n=16.

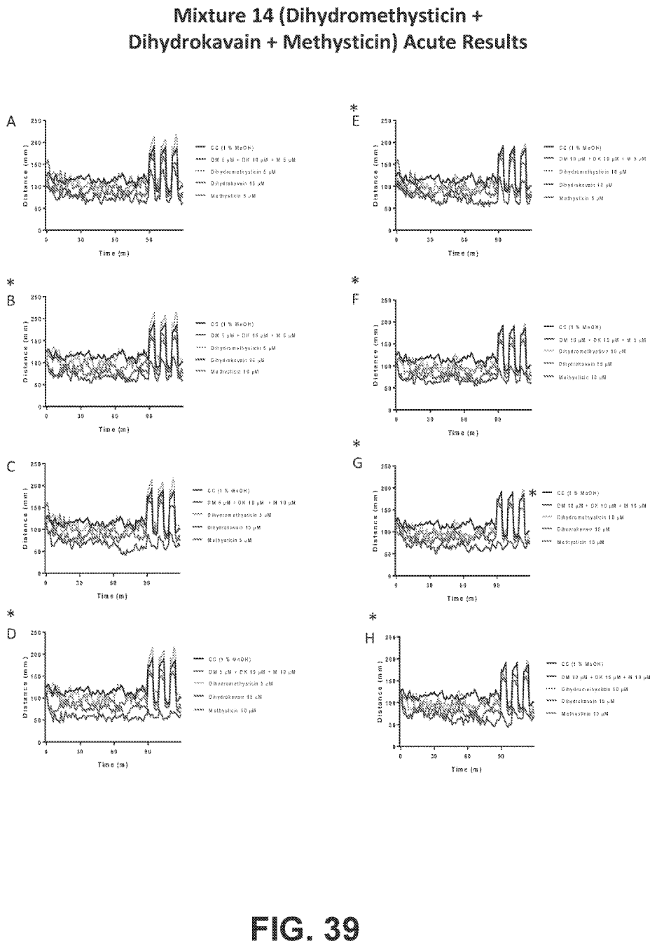

FIG. 39 shows concentration response profile following acute exposure to multiple concentrations of Mixture 14 (Dihydromethysticin, Dihydrokavain and Methysticin) and control treated larvae (1% MeOH). Distance traveled is presented in millimeters and time in minutes. Control n=32, treated n=16.

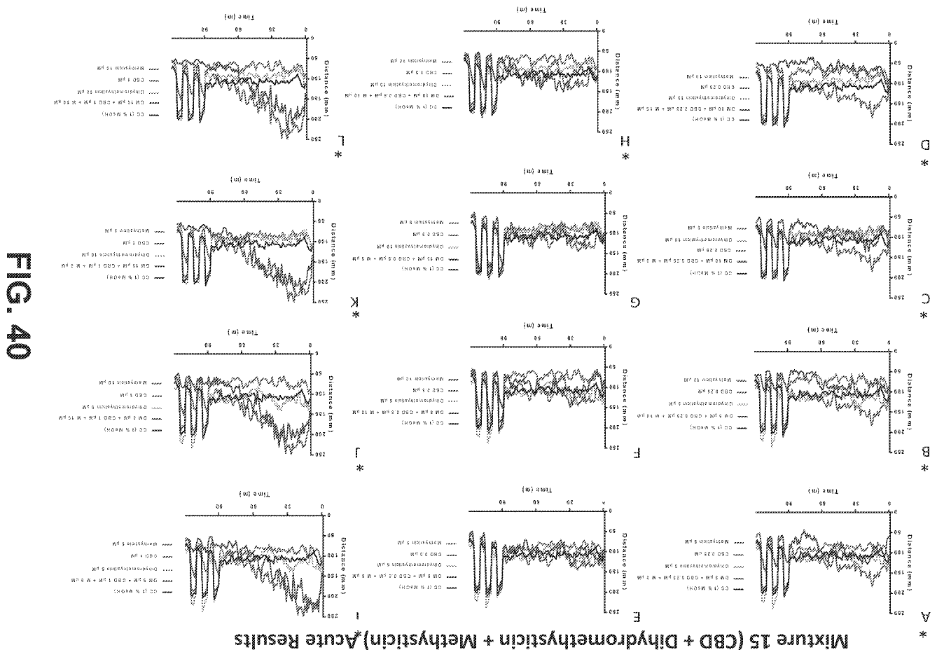

FIG. 40 shows concentration response profile following acute exposure to multiple concentrations of Mixture 15 (CBD, Methysticin and Dihydromethysticin) and control treated larvae (1% MeOH). Distance traveled is presented in millimeters and time in minutes. Control n=32, treated n=16.

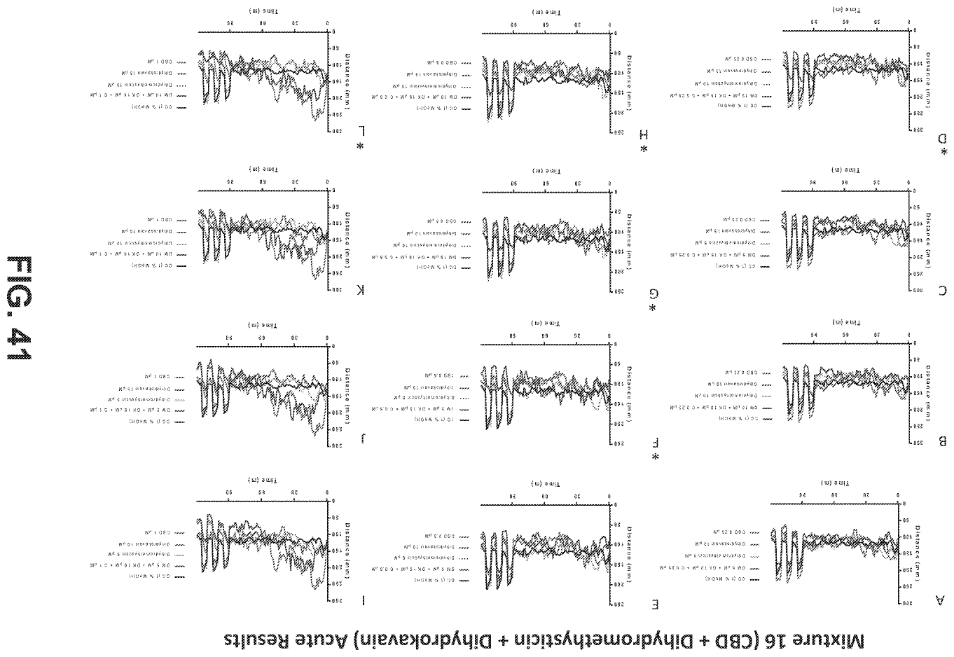

FIG. 41 shows concentration response profile following acute exposure to multiple concentrations of Mixture 16 (CBD, Methysticin and Dihydromethysticin) and control treated larvae (1% MeOH). Distance traveled is presented in millimeters and time in minutes. Control n=32, treated n=16.

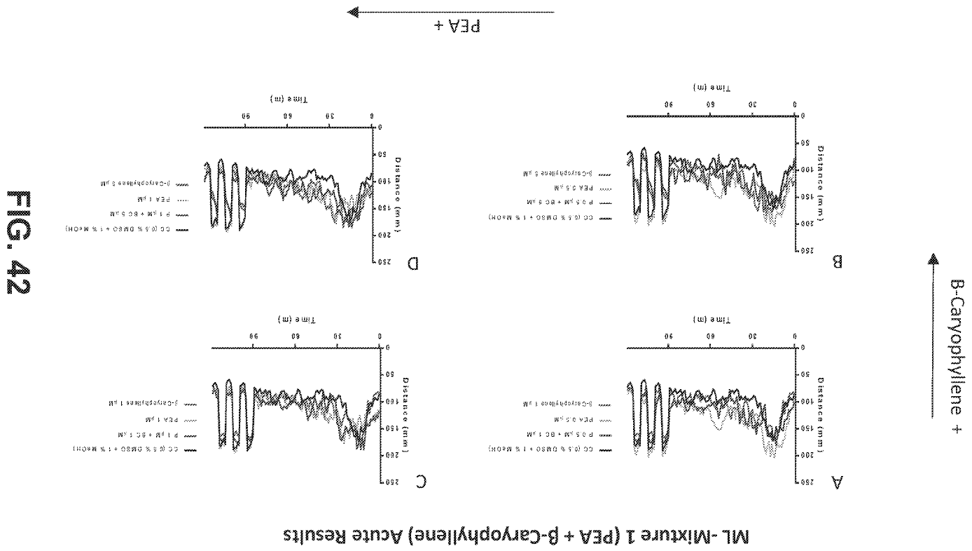

FIG. 42 shows concentration response profile following acute exposure to multiple concentrations of ML-Mixture 1 (PEA and B-Caryophyllene) and control treated larvae (0.5% DMSO+1% MeOH). Distance traveled is presented in millimeters and time in minutes. Control n=32, treated n=16.

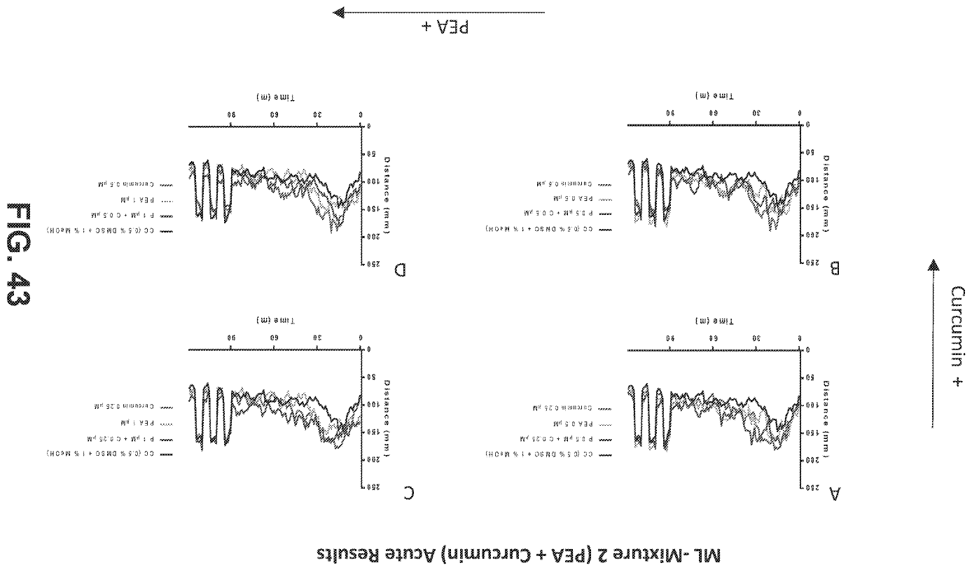

FIG. 43 shows concentration response profile following acute exposure to multiple concentrations of ML-Mixture 1 (PEA and Curcumin) and control treated larvae (0.5% DMSO+1% MeOH). Distance traveled is presented in millimeters and time in minutes. Control n=32, treated n=16.

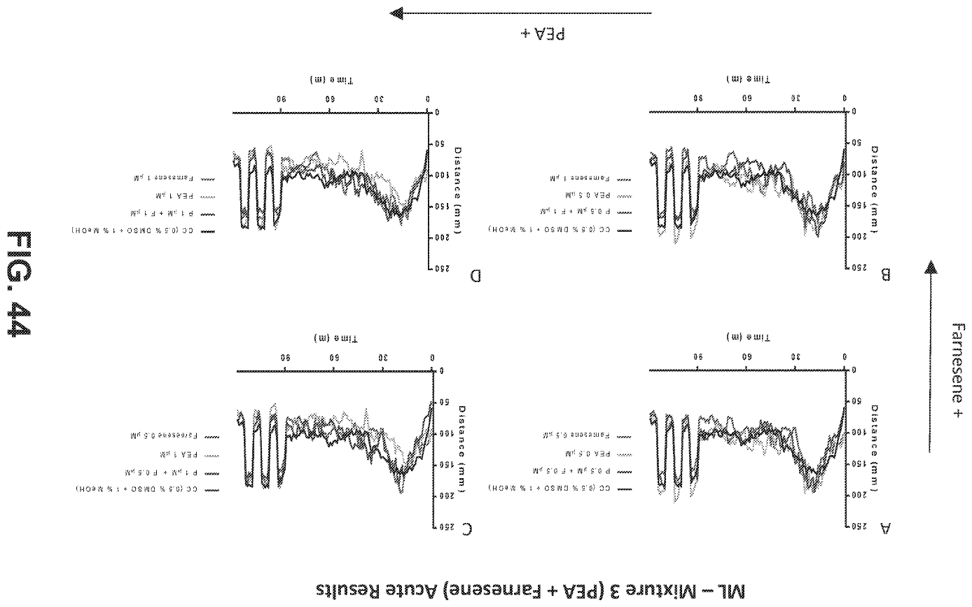

FIG. 44 shows concentration response profile following acute exposure to ML-Mixture 3, concentration matched pure PEA and Farnesene and control treated larvae. (0.5% DMSO+1% MeOH). Distance traveled is presented in millimeters and time in minutes. Each trace represents an n=24.

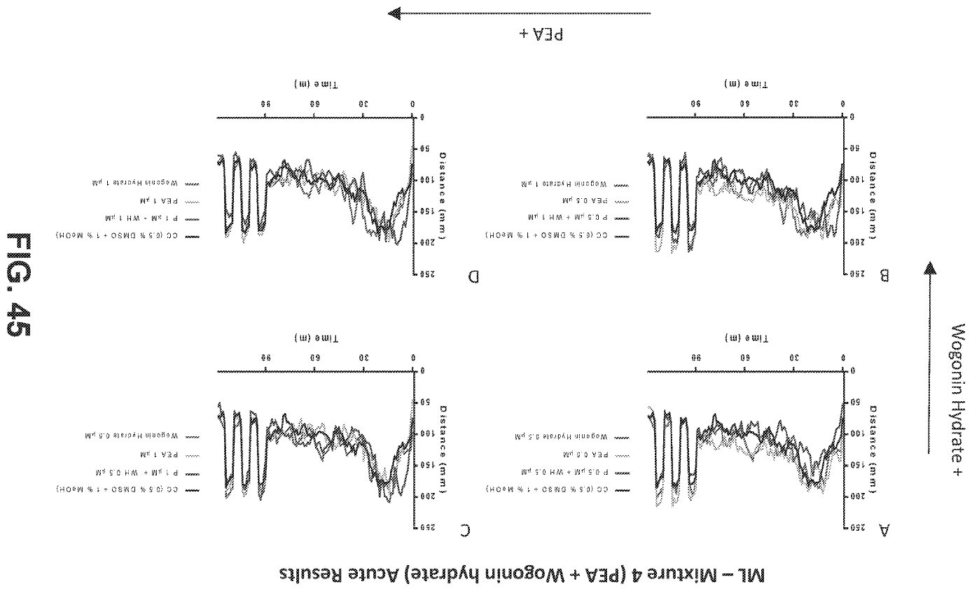

FIG. 45 shows concentration response profile following acute exposure to ML-Mixture 4, concentration matched pure PEA and Wogonin hydrate and control treated larvae. (0.5% DMSO+1% MeOH). Distance traveled is presented in millimeters and time in minutes. Each trace represents an n=24.

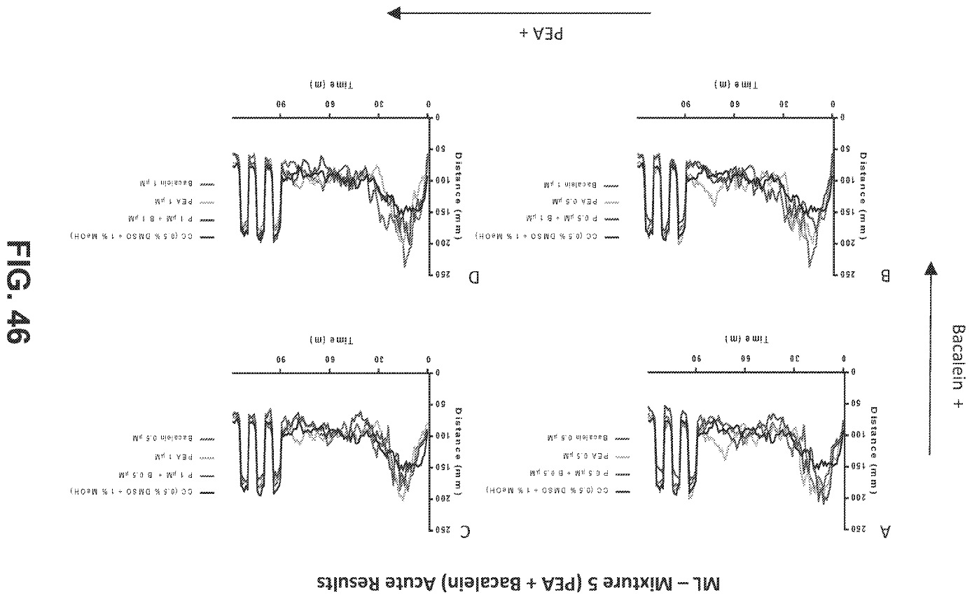

FIG. 46 shows concentration response profile following acute exposure to ML-Mixture 5, concentration matched pure PEA and Bacalein and control treated larvae. (0.5% DMSO+1% MeOH). Distance traveled is presented in millimeters and time in minutes. Each trace represents an n=24.

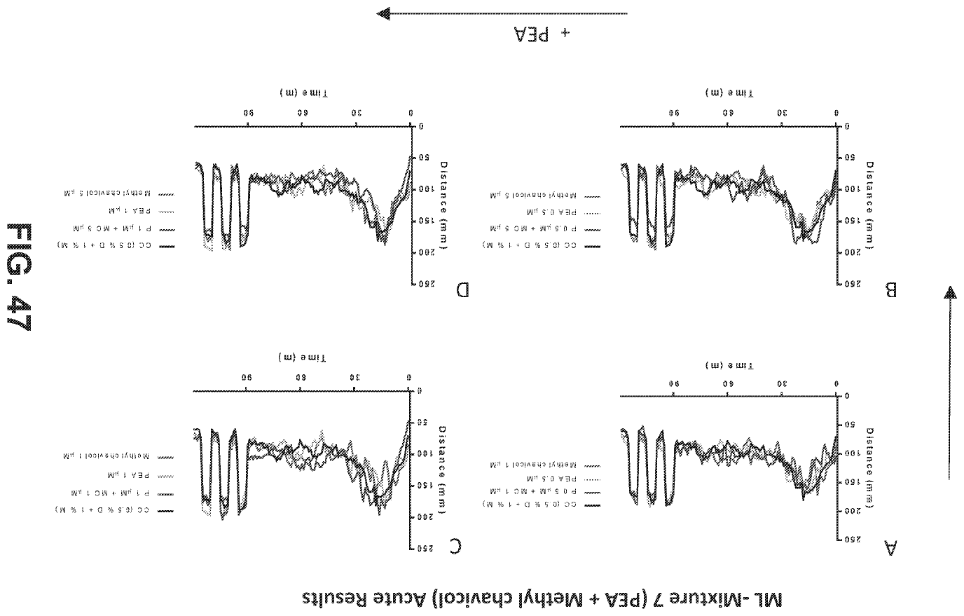

FIG. 47 shows concentration response profile following acute exposure to ML-Mixture 7, concentration matched pure PEA/Methyl chavicol and control treated larvae (0.5% DMSO1% MeOH). Distance traveled is presented in millimeters and time in minutes. Each trace represents an n=24.

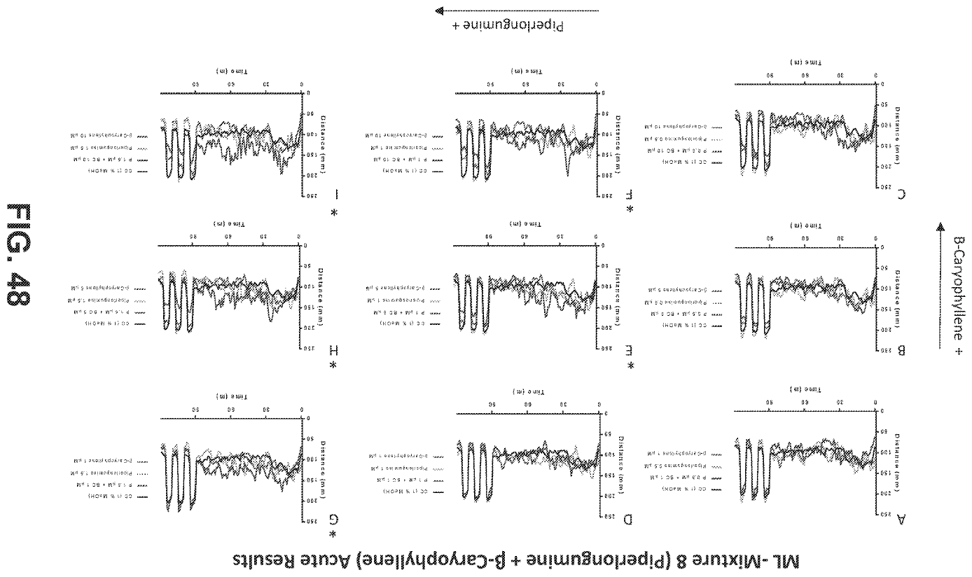

FIG. 48 shows concentration response profile following acute exposure to ML-Mixture 8, concentration matched pure Piperlongumine/B-Caryophyllene and control treated larvae (1% MeOH). Distance traveled is presented in millimeters and time in minutes. Each trace represents an n=24.

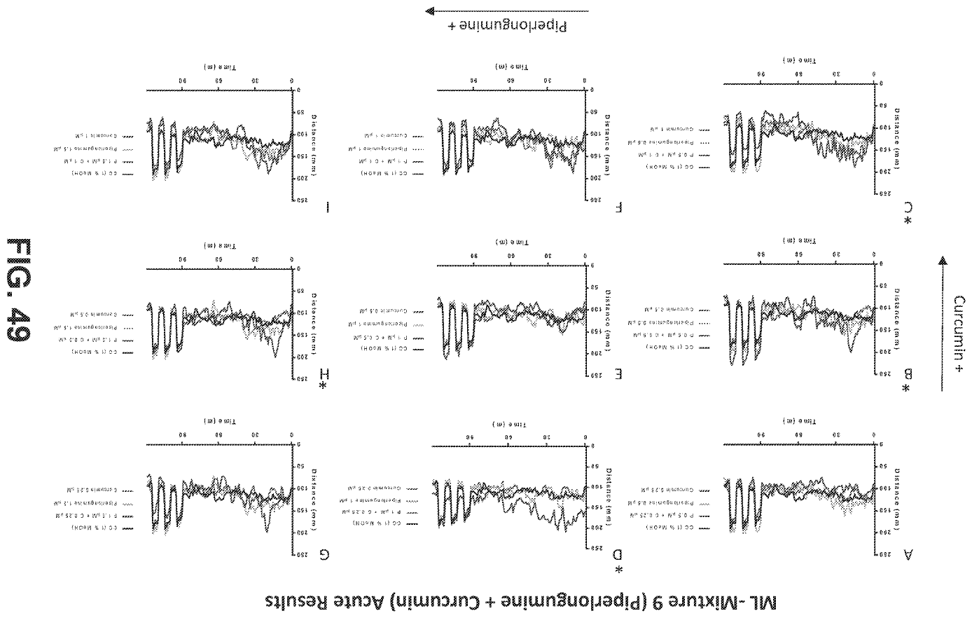

FIG. 49 shows concentration response profile following acute exposure to ML-Mixture 9, concentration matched pure Piperlongumine/Curcumin and control treated larvae (1% MeOH). Distance traveled is presented in millimeters and time in minutes. Each trace represents an n=24.

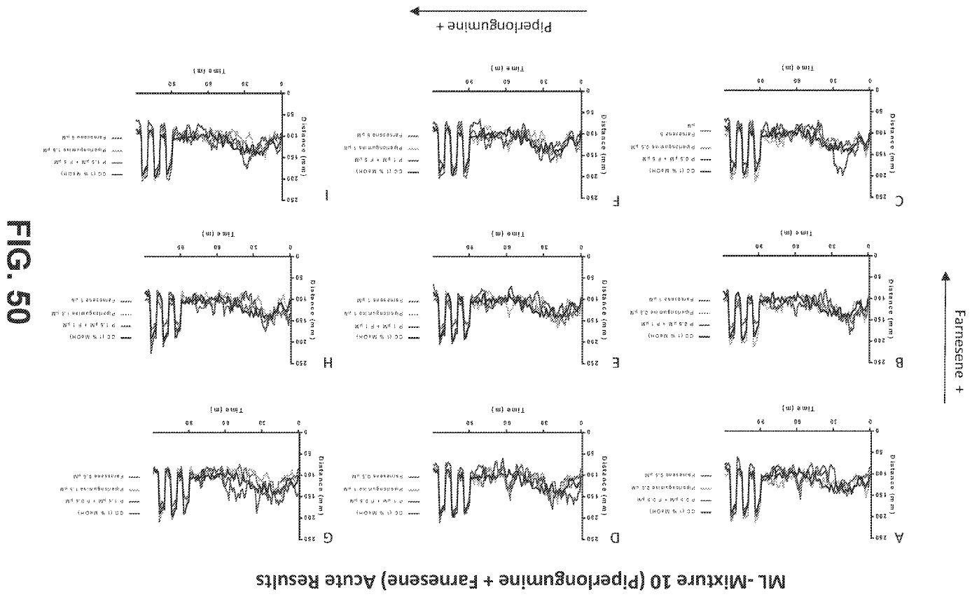

FIG. 50 shows concentration response profile following acute exposure to ML-Mixture 10, concentration matched pure Piperlongumine/Farnesene and control treated larvae (1% MeOH). Distance traveled is presented in millimeters and time in minutes. Each trace represents an n=24.

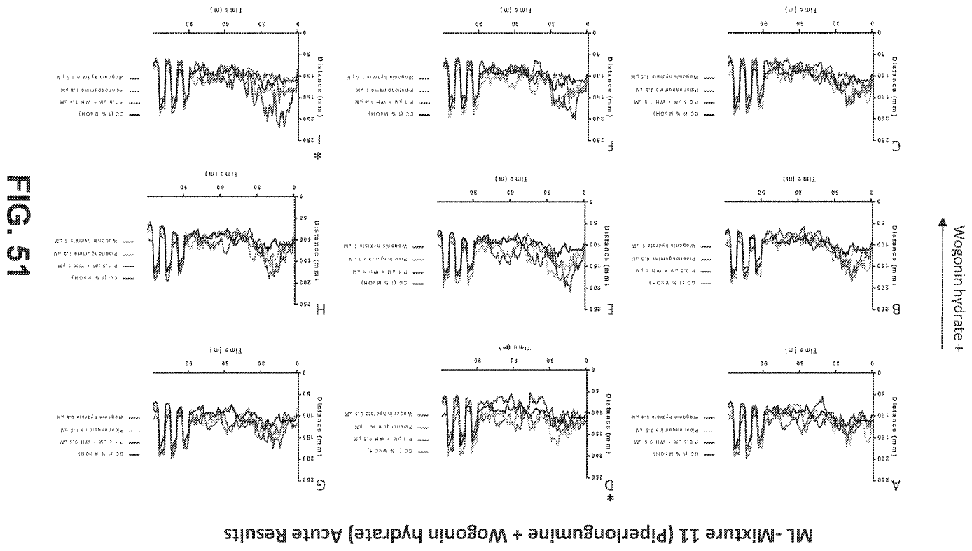

FIG. 51 shows concentration response profile following acute exposure to ML-Mixture 10, concentration matched pure Piperlongumine/Farnesene and control treated larvae (1% MeOH). Distance traveled is presented in millimeters and time in minutes. Each trace represents an n=24.

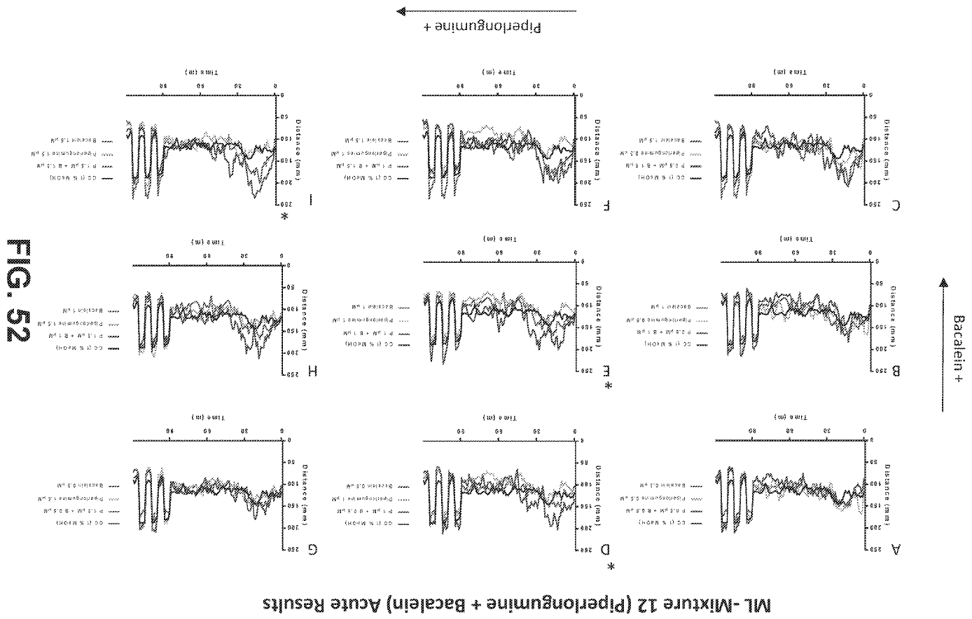

FIG. 52 shows concentration response profile following acute exposure to ML-Mixture 12, concentration matched pure Piperlongumine/Bacalein and control treated larvae (1% MeOH). Distance traveled is presented in millimeters and time in minutes. Each trace represents an n=24.

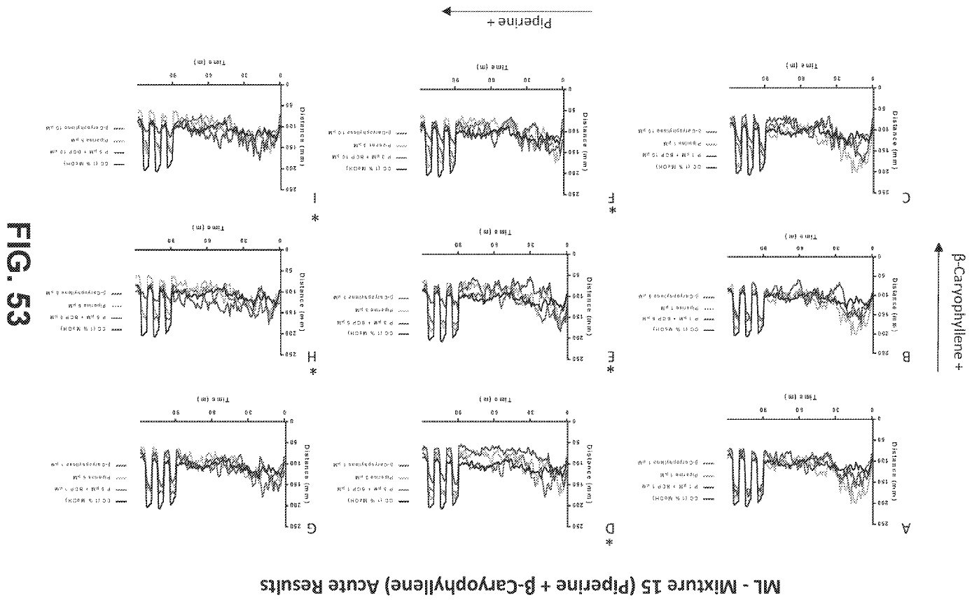

FIG. 53 shows concentration response profile following acute exposure to ML-Mixture 15, concentration matched pure Piperine/B-Caryophyllene and control treated larvae (1% MeOH). Distance traveled is presented in millimeters and time in minutes. Each trace represents an n=24.

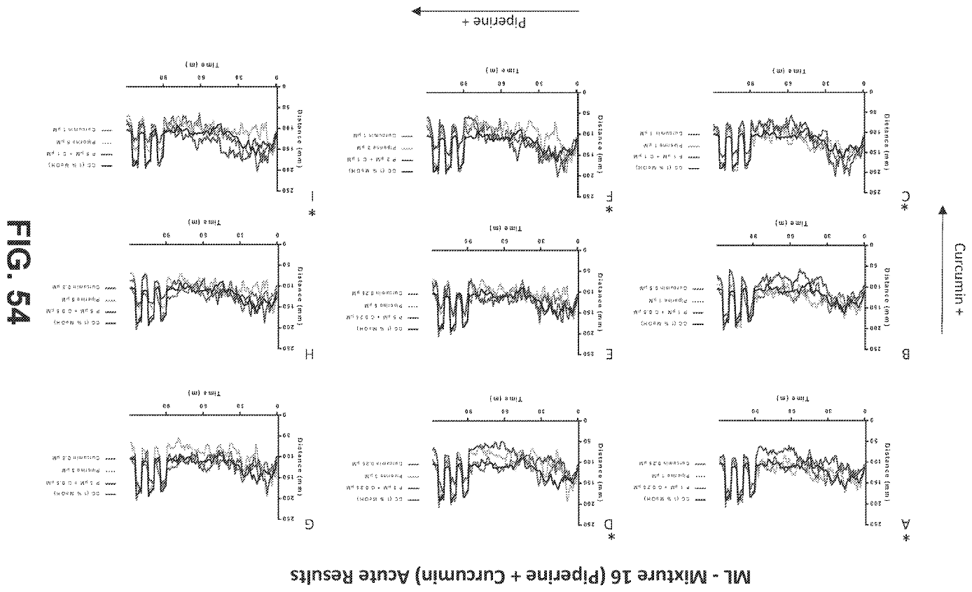

FIG. 54 shows concentration response profile following acute exposure to ML-Mixture 16, concentration matched pure Piperine/Curcumin and control treated larvae (1% MeOH). Distance traveled is presented in millimeters and time in minutes. Each trace represents an n=24.

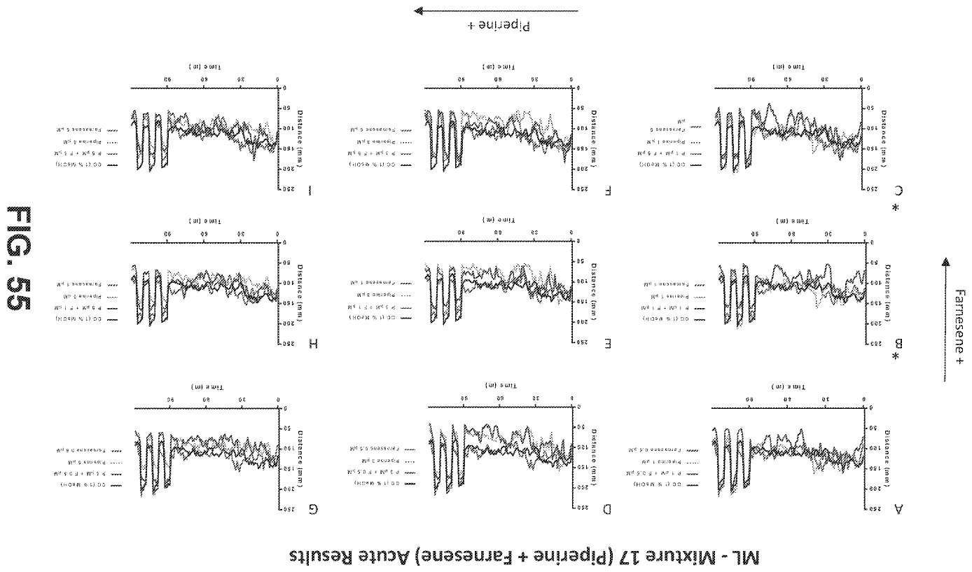

FIG. 55 shows concentration response profile following acute exposure to ML-Mixture 17, concentration matched pure Piperine/Farnesene and control treated larvae (1% MeOH). Distance traveled is presented in millimeters and time in minutes. Each trace represents an n=24.

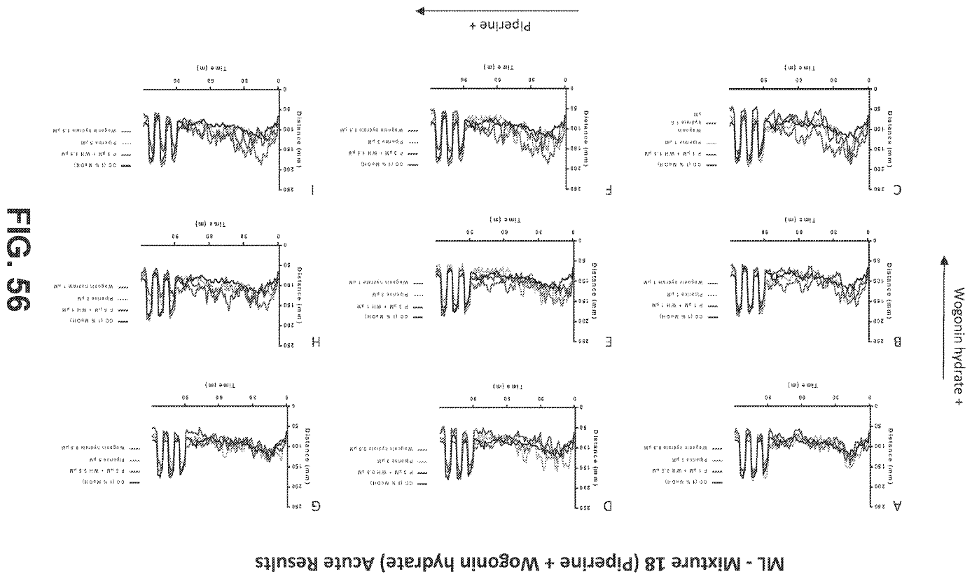

FIG. 56 shows concentration response profile following acute exposure to ML-Mixture 18, concentration matched pure Piperine/Wogonin hydrate and control treated larvae (1% MeOH). Distance traveled is presented in millimeters and time in minutes. Each trace represents an n=24.

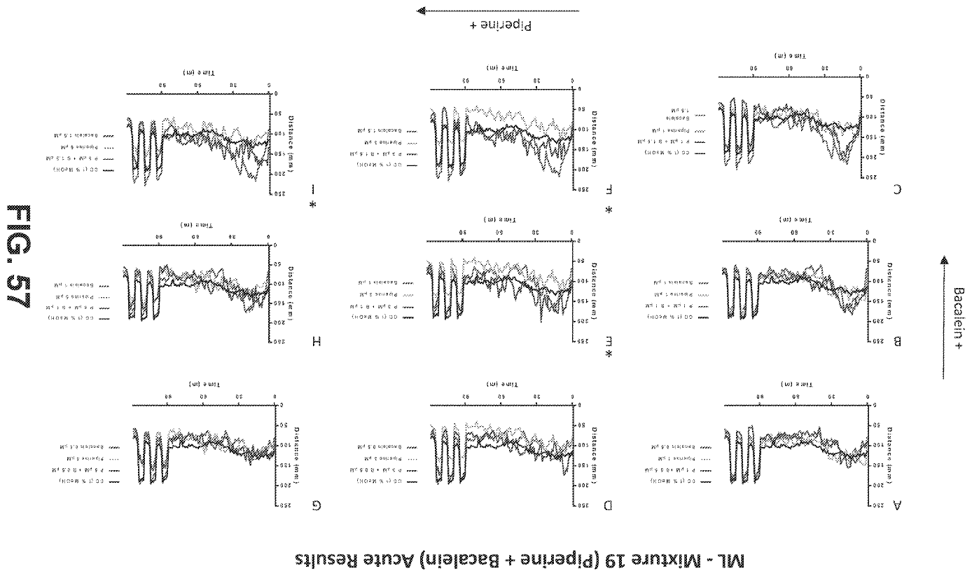

FIG. 57 shows concentration response profile following acute exposure to ML-Mixture 18, concentration matched pure Piperine/Bacalein and control treated larvae (1% MeOH). Distance traveled is presented in millimeters and time in minutes. Each trace represents an n=24.

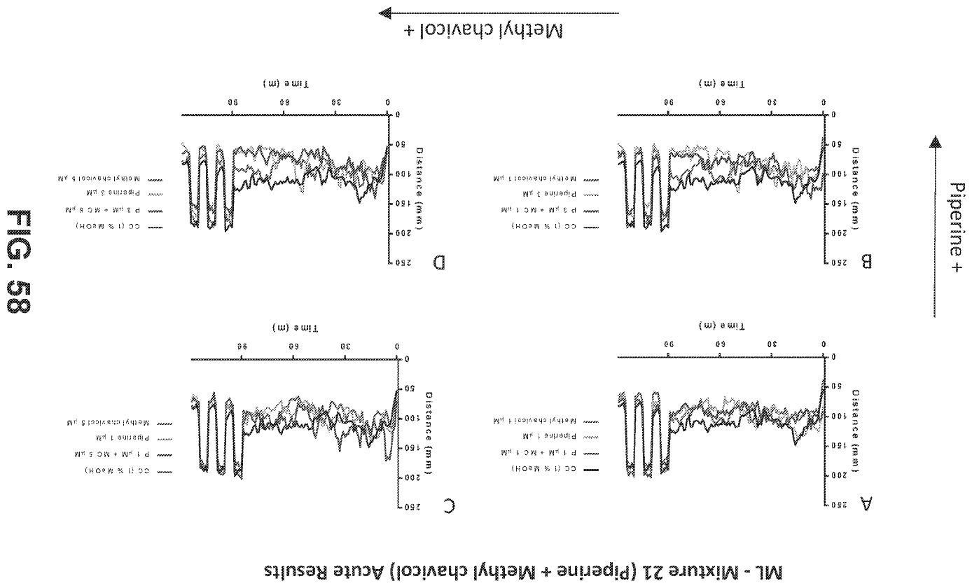

FIG. 58 shows concentration response profile following acute exposure to ML-Mixture 21, concentration matched pure Piperine/Methyl chavicol and control treated larvae (1% MeOH). Distance traveled is presented in millimeters and time in minutes. Each trace represents an n=24.

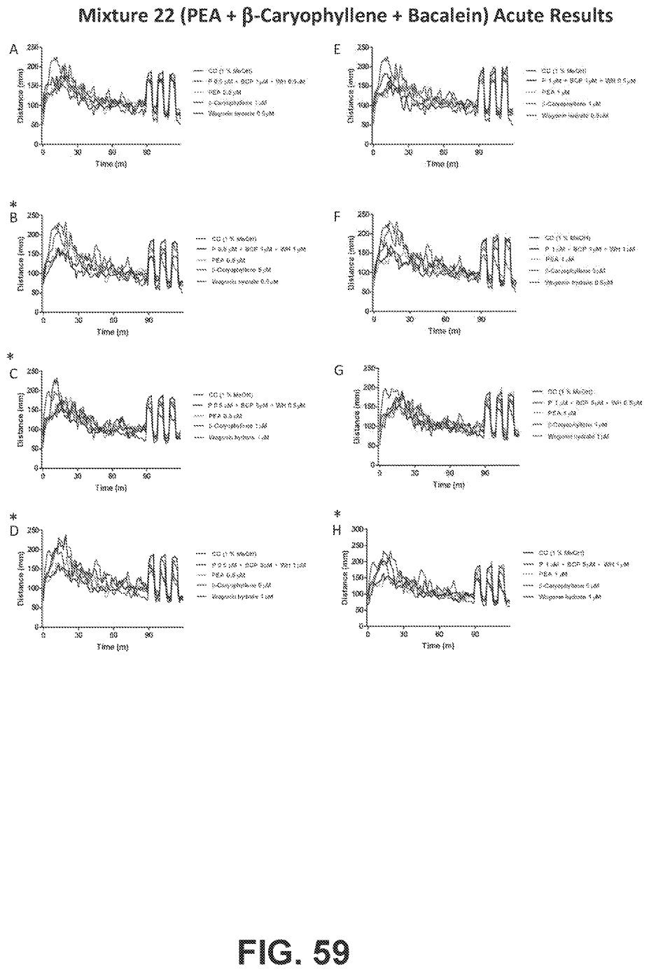

FIG. 59 shows concentration response profile following acute exposure to multiple concentrations of Mixture 22 (PEA, B-Caryophyllene, and Wogonin) and control treated larvae (1% MeOH). Distance traveled is presented in millimeters and time in minutes. Control n=64, treated n=16.

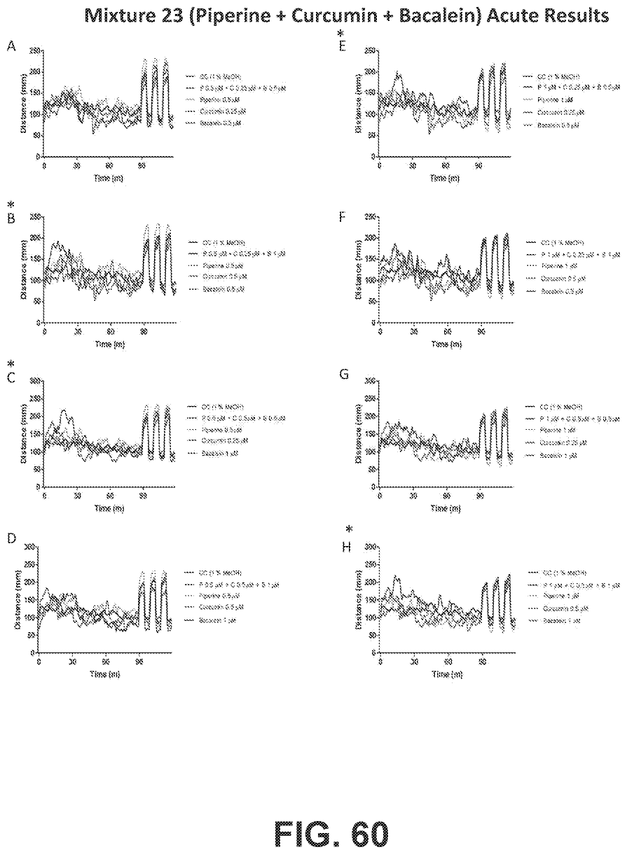

FIG. 60 shows concentration response profile following acute exposure to multiple concentrations of Mixture 23 (Piperine, Curcumin and Bacalein) and control treated larvae (1% MeOH). Distance traveled is presented in millimeters and time in minutes. Control n=64, treated n=16.

FIG. 61A shows a Thigmotaxis Model system in a well plate for testing stress in zebrafish larvae. (A) Light/dark program used in behavioral experiment. (B) 24 well plate grid with zone size and placement used in behavioral experiment.

FIG. 61B shows a Thigmotaxis Model with Larvae loaded in 24 well plates @ 48 hpf. Treated at ~125 hpf and immediately transferred to a tracking system. Acclimated for 6 mins in light (data not used for reporting) and tracked 4 mins in dark. The following formulas were used for this Thigmotaxis Model of FIG. 61B:

$$Thigmotaxis ( \% TDMinouterzone ) = [ \frac{TDMinouter}{TDMouier + inner} ] \times 100$$

$$Thigmotaxis ( \% time inouterzone ) = [ \frac{timeouter}{testduration ( i . e . 240 s )} ] \times 100$$

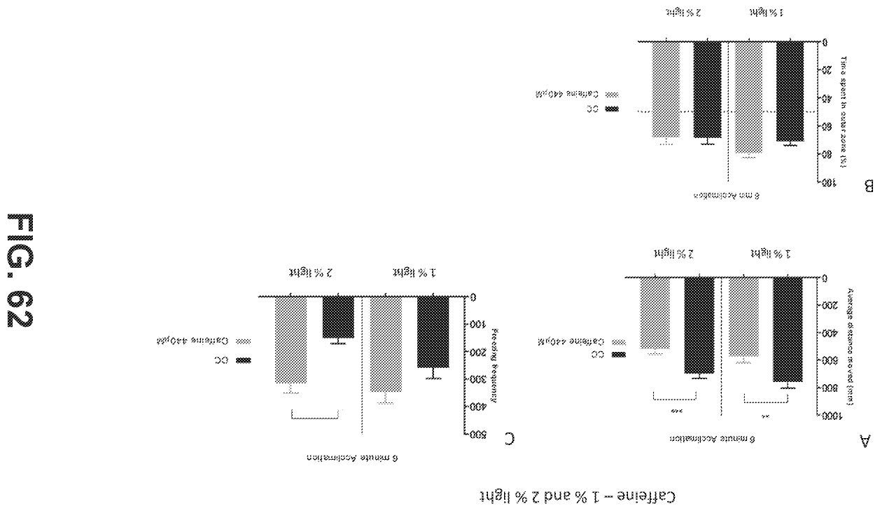

FIG. 62 shows larval activity following exposure to multiple concentrations of Mixture 2. (A) Average distance moved. (B) Freezing frequency (inactivity). (C) % of time spent in the well outer zone. Activity was compared to control to controls using One-way ANOVA and a Dunnett's multiple comparison test. Control n=~190, treated n=~24.

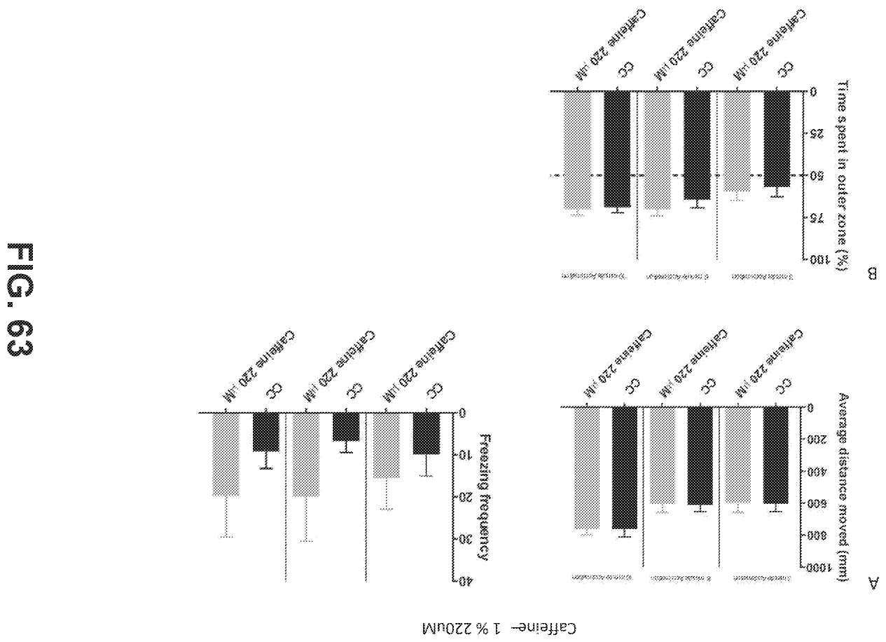

FIG. 63 shows larval activity following exposure to multiple concentrations of Mixture 2. (A) Average distance moved. (B) Freezing frequency (inactivity). (C) % of time spent in the well outer zone. Activity was compared to control to controls using One-way ANOVA and a Dunnett's multiple comparison test. Control n=~190, treated n=~24.

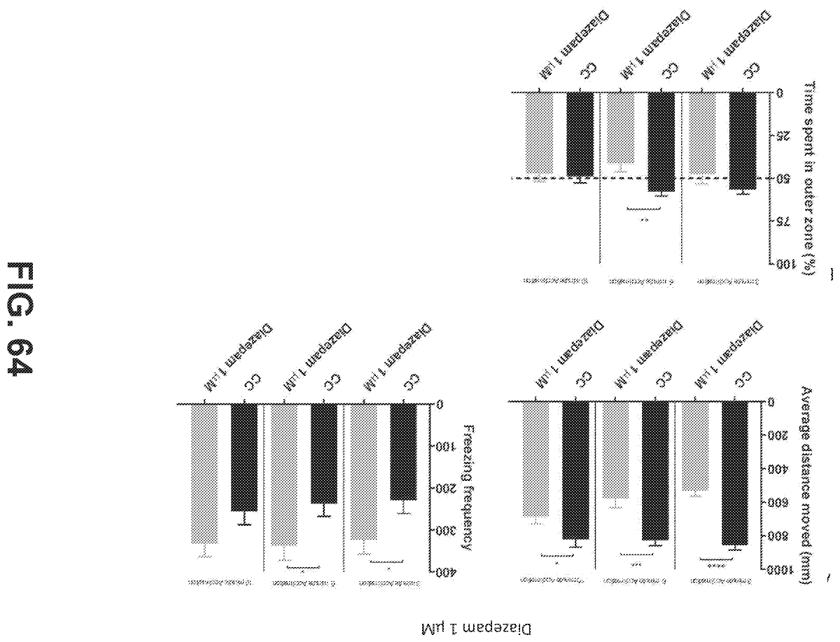

FIG. 64 shows larval activity following exposure to 1 UM Diazepam. (A) Average distance moved. (B) Freezing frequency (inactivity). (C) % of time spent in the well outer zone. Activity was compared to control to controls using One-way ANOVA and a Dunnett's multiple comparison test. Control n=~190, treated n=~24.

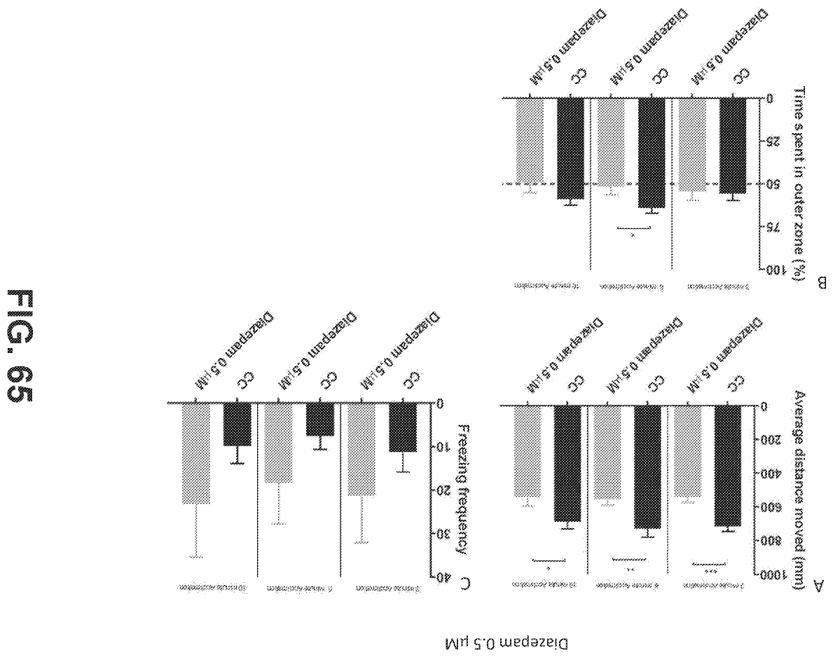

FIG. 65 shows larval activity following exposure to 0.5 UM Diazepam. (A) Average distance moved. (B) Freezing frequency (inactivity). (C) Average fast activity (D) % of time spent in the well outer zone. Activity was compared to control to controls using One-way ANOVA and a Dunnett's multiple comparison test. Control n=~190, treated n=~24.

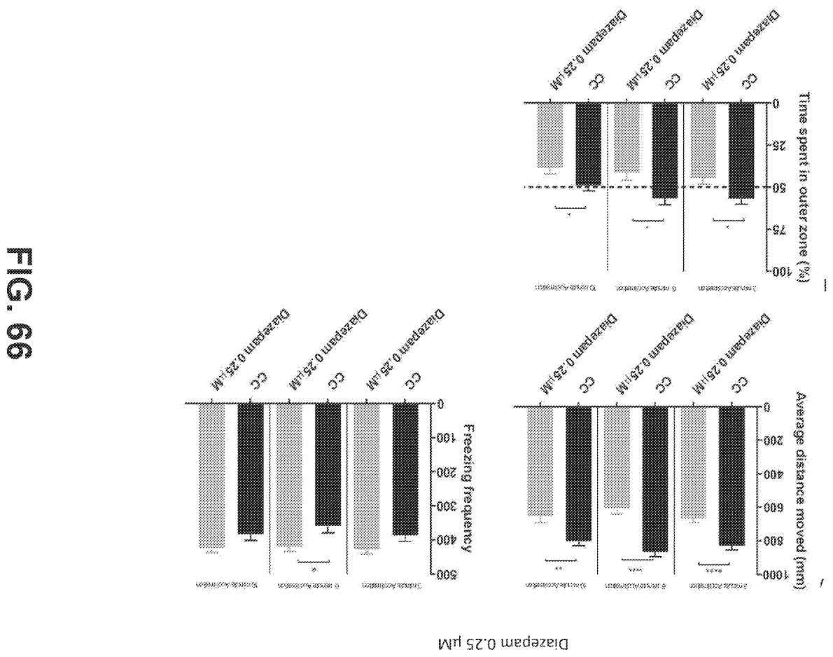

FIG. 66 shows larval activity following exposure to 0.25 UM Diazepam. (A) Average distance moved. (B) % of time spent in the well outer zone (C) Freezing frequency (inactivity). Activity was compared to control to controls using One-way ANOVA and a Dunnett's multiple comparison test. Control n=~190, treated n=~24.

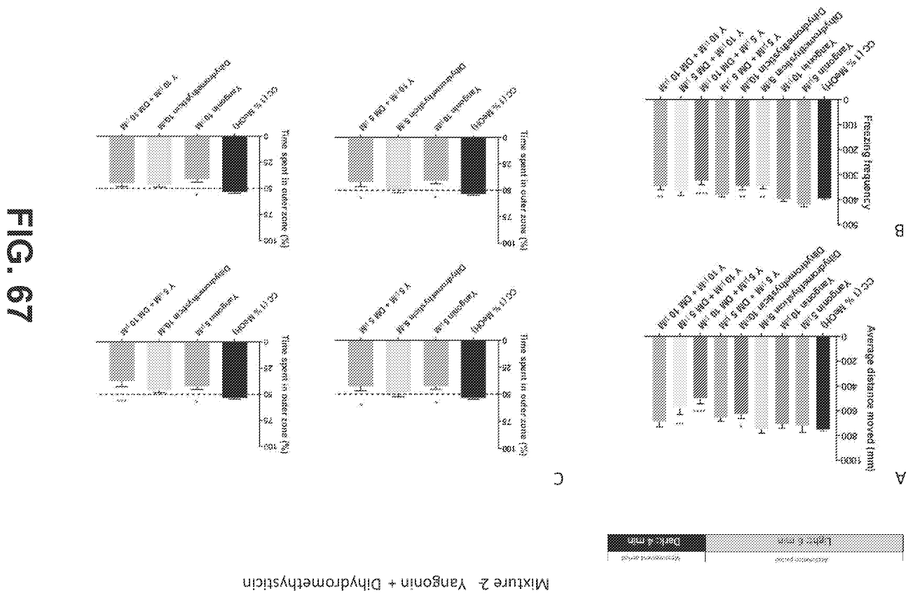

FIG. 67 shows larval activity following exposure to multiple concentrations of Mixture 2. (A) Average distance moved. (B) Freezing frequency (inactivity). (C) Average fast activity (D) % of time spent in the well outer zone. Activity was compared to control to controls using One-way ANOVA and a Dunnett's multiple comparison test. Control n=~190, treated n=~24.

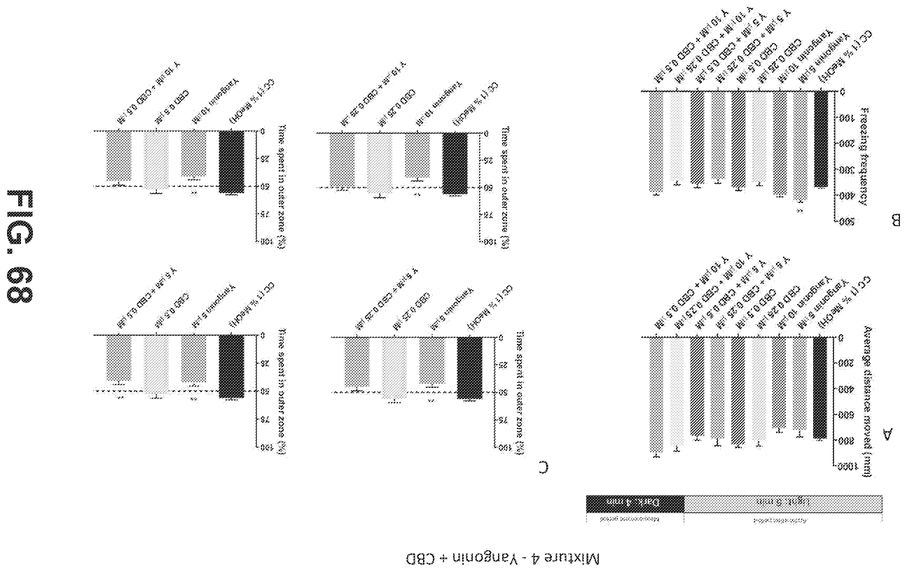

FIG. 68 shows larval activity following exposure to multiple concentrations of Mixture 4. (A) Average distance moved. (B) Freezing frequency (inactivity). (C) Average fast activity (D) % of time spent in the well outer zone. Activity was compared to control to controls using One-way ANOVA and a Dunnett's multiple comparison test. Control n=~190, treated n=~24.

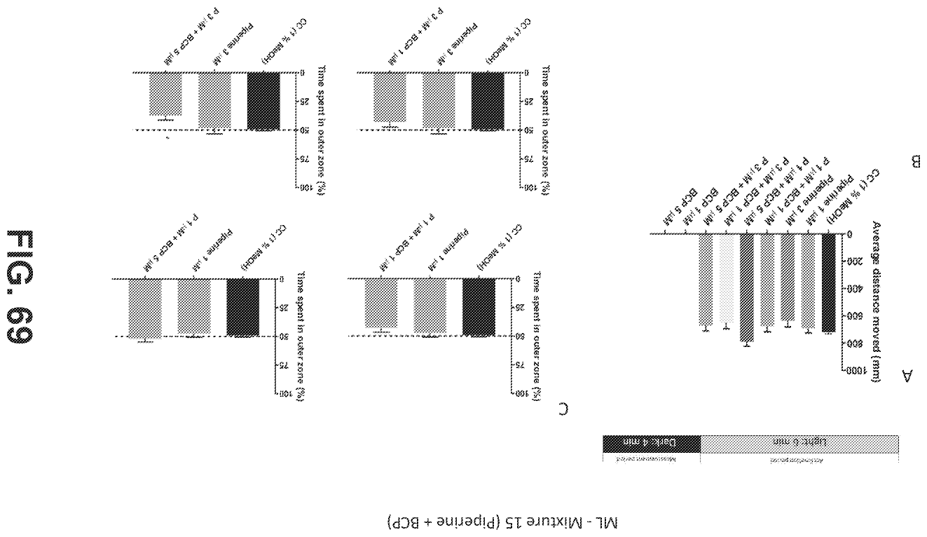

FIG. 69 shows larval activity following exposure to multiple concentrations of ML-Mixture 15. (A) Average distance moved. (B) Freezing frequency (inactivity). (C) Average fast activity (D) % of time spent in the well outer zone. Activity was compared to control to controls using One-way ANOVA and a Dunnett's multiple comparison test. Control n=~190, treated n=~24.

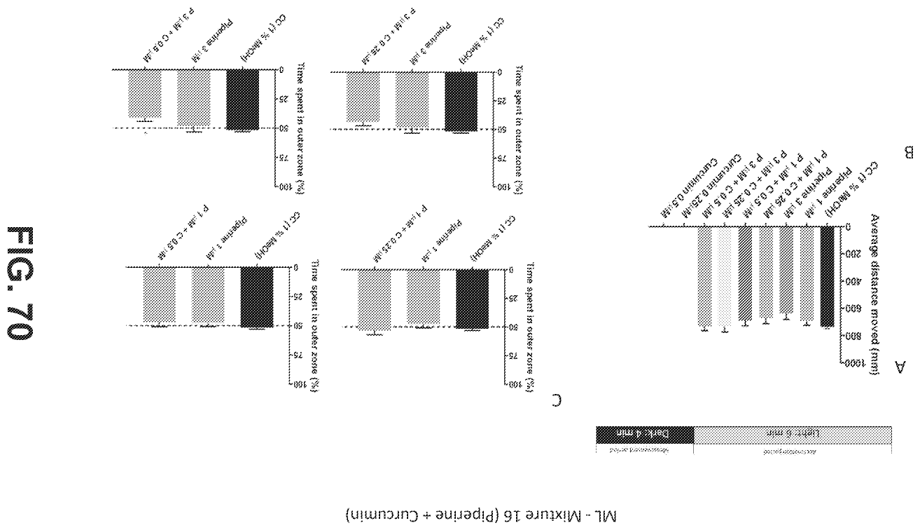

FIG. 70 shows larval activity following exposure to multiple concentrations of ML-Mixture 16. (A) Average distance moved. (B) Freezing frequency (inactivity). (C) Average fast activity (D) % of time spent in the well outer zone. Activity was compared to control to controls using One-way ANOVA and a Dunnett's multiple comparison test. Control n=~190, treated n=~24.

Below is a summary of the representative test results from the thigmotaxis assay.

| | | | | Time | |
|---|---|---|---|---|---|
| | | | | Spent | Thigmotaxic |
| | | | Average | in | reactivity |
| | | | Distance | Outer | indicates |
| Mixture | | Constituent | Moved | Zone | anxiolytic |
| # | Constituents | Concentrations | (mm) | (%) | potential |
| 2 | Yangonin | 5 | [symbol] | [symbol] | Y |
| | | 10 | [symbol] | [symbol] | Y |
| | Dihydro- | 5 | [symbol] | [symbol] | N |
| | methysticin | 10 | [symbol] | [symbol] | N |
| | Y + DM | 5 + 5 | [symbol] | [symbol] | Y |
| | | 5 + 10 | [symbol] | [symbol] | N |
| | | 10 + 5 | [symbol] | [symbol] | N |
| | | 10 + 10 | [symbol] | [symbol] | N |
| 4 | Yangonin | 5 | [symbol] | [symbol] | Y |
| | | 10 | [symbol] | [symbol] | Y |
| | CBD | 0.25 | [symbol] | [symbol] | N |
| | | 0.5 | [symbol] | [symbol] | N |
| | Y + CBD | 5 + 0.25 | [symbol] | [symbol] | N |
| | | 5 + 0.5 | [symbol] | [symbol] | Y |
| | | 10 + 0.25 | [symbol] | [symbol] | N |
| | | 10 + 0.5 | [symbol] | [symbol] | N |
| 15 | Piperine | 1 | [symbol] | [symbol] | N |
| | | 3 | [symbol] | [symbol] | N |
| | β- | 1 | N/A | N/A | N/A |
| | caryophyllene | 5 | N/A | N/A | N/A |
| | Pip + BCP | 1 + 1 | [symbol] | [symbol] | N |
| | | 1 + 5 | [symbol] | [symbol] | N |
| | | 3 + 1 | [symbol] | [symbol] | N |
| | | 3 + 5 | [symbol] | [symbol] | Y |
| 16 | Piperine | 1 | [symbol] | [symbol] | N |
| | | 3 | [symbol] | [symbol] | N |
| | Curcumin | 0.25 | N/A | N/A | N/A |
| | | 0.5 | N/A | N/A | N/A |
| | Pip + Curc | 1 + 0.25 | [symbol] | [symbol] | N |
| | | 1 + 0.5 | [symbol] | [symbol] | N |
| | | 3 + 0.25 | [symbol] | [symbol] | N |
| | | 3 + 0.5 | [symbol] | [symbol] | Y |
| | Piper- | 1.5 | [symbol] | [symbol] | N |
| | longumine | 3 | [symbol] | [symbol] | N |
| | Farnesene | 5 | [symbol] | [symbol] | N |
| | | 10 | [symbol] | [symbol] | N |
| | Methysticin | 5 | [symbol] | [symbol] | N |
| | | 10 | [symbol] | [symbol] | N |

The foregoing has described the principles, embodiments and modes of operation of the present invention. However, the invention should not be construed as being limited to the particular embodiments discussed. The above described embodiments should be regarded as illustrative rather than restrictive, and it should be appreciated that variations may be made in those embodiments by workers skilled in the art without departing from the scope of the present invention as defined by the following claims.

1. (canceled)

2. A method of treating stress, anxiety, and anxiety disorders in a human, comprising administering to a human in need of said treatment a predetermined effective amount of two phytochemicals obtained as at least one of naturally as a purified ingredient or synthetically produced as an identical homologue, wherein the treatment is a double ingredient therapy comprised of at least two compounds in a ratio-controlled mixture, wherein:
the first and second phytochemical is selected from Yangonin (Y), Methysticin (M), Dihydromethysticin (DM), Cannabidiol (CBD), Dihydrokavain (DK), Piperlongumine (PL), Piperine (P), Bacalein (B), beta-Caryophyllene (BCP), Farnesene (F), Curcumin (CM), and Wogonin (W); and
wherein the first and second phytochemicals are not the same.

3. The method of claim 2, wherein the at least two compounds in a ratio-controlled mixture comprises Yangonin (Y)+Methysticin (M) as at least one of GBS.001F as 5.0 μM Y+10.0 μM M (1:2 ratio) or GBS.001I or 10.0 μM Y+10.0 μM M (1:1 ratio).

4. The method of claim 2, wherein the at least two compounds in a ratio-controlled mixture comprises Yangonin (Y)+Dihydromethysticin (DM) as at least one of GBS.002E as 5.0 Y+10.0 DM (1:2 ratio), or GBS.002F as 10.0 μM Y+10.0 μM DM (1:1 ratio), or GBS.002G as 10.0 μM Y+1.0 μM DM (10:1 ratio), or GBS.002H as 10.0 μM Y+5.0 μM DM (2:1 ratio), or GBS.002I as 10.0 μM Y+10.0 μM DM (1:1 ratio).

5. The method of claim 2, wherein the at least two compounds in a ratio-controlled mixture comprises Yangonin (Y)+Cannabidiol (CBD) as at least one of GBS.004G as 5.0 μM Y+1.0 μM CBD (5:1 ratio), or GBS.004K as 10.0 μM Y+1.0 μM CBD (10:1 ratio), or GBS.002L as 10.0 μM Y+1.5 μM CBD (10:15 ratio).

6. The method of claim 2, wherein the at least two compounds in a ratio-controlled mixture comprises Dihydromethysticin (DM)+Methysticin (M) as at least one of GBS.005C as 1.0 μM DM+10.0 μM M (1:10 ratio), or GBS.005D as 5.0 μM DM+1.0 μM M (5.1 ratio), or GBS.005F as 5.0 AM DM+10.0 μM M (1:2 ratio), GBS.005H as 10.0 μM DM+5.0 μM M (2.1 ratio), GBS.005H as 10.0 μM DM+10.0 μM M (1:1 ratio).

7. The method of claim 2, wherein the at least two compounds in a ratio-controlled mixture comprises Dihydrokavain (DK)+Methysticin (M) as at least one of GBS.006G as 10.0 DK+10.0 1-1M M (1:1 ratio), or GBS.0061 as 25.0 1-1M DK+10.0 1-1M M (25.1 ratio).

8. The method of claim 2, wherein the at least two compounds in a ratio-controlled mixture comprises Cannabidiol (CBD)+Methysticin (M) as at least one of GBS.007K as 1.0 μM CBD+10.0 μM M (1:10 ratio), or GBS.007L as 1.5 μM CBD+10.0 μM M (1.5:10 ratio).

9. The method of claim 2, wherein the at least two compounds in a ratio-controlled mixture comprises Dihydromethysticin (DM)+Dihydrokavain (DK) as at least one of GBS.008C as 1.0 μM DM+25.0 μM DK (1:25 ratio), GBS.008F as 5.0 μM DM+25.0 μM DK (1:5 ratio), or GBS.008F as 10.0 μM DM+25.0 μM DK (19.5 ratio).

10. The method of claim 2, wherein the at least two compounds in a ratio-controlled mixture comprises Dihydromethysticin (DM)+Cannabidiol (CBD) as at least one of GBS.009E as 5.0 μM DM+0.25 M CBD (20.1 ratio), GBS.0091 as 10.0 μM DM+0.25 μM CBD (40:1 ratio), or GBS.009J as 10.0 μM DM+1.0 μM CBD (10:1 ratio).

11. The method of claim 2, wherein the at least two compounds in a ratio-controlled mixture comprises Dihydrokavain (DK)+Cannabidiol (CBD) as at least one of GBS.010H as 10.0 μM DK+1.5 μM CBD (10:1.5 ratio), GBS.010I as 25.0 μM DK+0.25 μM CBD (100:1 ratio), or GBS.010J as 25.0 μM DK+0.5 μM CBD (50:1 ratio), GBS.010K as 25.0 μM DK+1.0 μM CBD (25:1 ratio), GBS.010L as 25.0 μM DK+1.5 μM CBD (25:1.5 ratio).

12. The method of claim 2, wherein the at least two compounds in a ratio-controlled mixture comprises Piperlongumine (PL)+Wogonin hydrate (WH) as at least one of GBS.ML011D as 1.0 μM PL+0.5 WH (2.1 ratio) or GBS.ML011I as 1.5 μM PL+1.5 μM WH (1:1 ratio).

13. (canceled)

14. The method of claim 2, wherein the at least two compounds in a ratio-controlled mixture comprises Piperlongumine (PL)+Bacalein (B) as at least one of GBS.ML012D as 1.0 μM PL+0.5 μM B (2.1 ratio) or GBS.ML012E as 1.0 μM PL+1.0 μM B (1:1 ratio).

15. The method of claim 2, wherein the at least two compounds in a ratio-controlled mixture comprises Piperine (P)+beta-Caryophyllene (BCP) as at least one of GBS.ML015D as 3.0 μM P+1.0 μM BCP (3:1 ratio), GBS.ML015E as 3.0 μM P+5.0 μM BCP (3:5 ratio), GBS.ML015F as 3.0 μM P+10.0 μM BCP (3:10 ratio), GBS.ML015H as 5.0 μM P+5.0 μM BCP (1:1 ratio), or GBS.ML015I as 5.0 μM P+10.0 μM BCP (1:2 ratio).

16. The method of claim 2, wherein the at least two compounds in a ratio-controlled mixture comprises Piperine (P)+Curcumin (CM) as at least one of GBS.ML016A as 10 μM P+0.25 μM CM (4:1 ratio), GBS.ML016C as 1.0 μM P+1.0 μM CM (1:1 ratio), GBS.ML016D as 3.0 μM P+0.25 μM CM (12:1 ratio), GBS.ML016F as 3.0 μM P+1.0 μM CM (3.1 ratio), or GBS.ML0161 as 5.0 μM P+1.0 μM CM (5.1 ratio).

17. The method of claim 2, wherein the at least two compounds in a ratio-controlled mixture comprises Piperine (P)+Farnesene (F) as at least one of GBS.ML016A as 1.0 μM P+1.0 μM F (1:1 ratio), or GBS.ML0161 as 1.0 μM P+5.0 μM F (1:5 ratio).

18. The method of claim 2, wherein the at least two compounds in a ratio-controlled mixture comprises Piperine (P)+Bacalein (BL) as at least one of GBS.ML019E as 3.0 μM P+1.0 μM BL (3:1 ratio), GBS.ML019F as 3.0 μM P+1.5 μM BL (3:1.5 ratio), or GBS.ML0191 as 5.0 μM P+1.5 UM BL (51.5 ratio).

19. A method of treating stress, anxiety, and anxiety disorders in a human, comprising administering to a human in need of said treatment a predetermined effective amount of three phytochemicals obtained as at least one of naturally as a purified ingredient or synthetically produced as an identical homologue, wherein the treatment is a triple ingredient therapy comprised of at least three compounds in a ratio-controlled mixture, wherein:
the first, second, and third phytochemical is selected from Yangonin (Y), Methysticin (M), Dihydromethysticin (DM), Cannabidiol (CBD), Dihydrokavain (DK), Piperlongumine (PL), PEA, Piperine (P), Bacalein (B), beta-Caryophyllene (BCP), Farnesene (F), Curcumin (CM), and Wogonin (W); and
wherein the first, second, and third phytochemicals are not the same.

20. The method of claim 19, wherein the at least three compounds in a ratio-controlled mixture comprises Yangonin (Y)+Dihydromethysticin (DM)+Methysticin (M) as at least one of GBS.011A as 5.0 μM Y+5.0 μM DM+5.0 μM M ratio), GBS.011B as 5.0 μM Y+5.0 μM DM+10.0 μM M (1:1:2 ratio), GBS.011C as 5.0 μM Y+10.0 μM DM+5.0 μM M (1:2:1 ratio), GBS.011D as 5.0 μM Y+10.0μ M DM+10.0 μM M (1:2:2 ratio), GBS.011E as 10.0 μM Y+5.0μ M DM+5.0 μM M (2:1:1 ratio), GBS.011F as 10.0 μM Y+5.0 μM DM+10.0 μM M (2:1:2 ratio), GBS.011G as 10.0 μM Y+10.0 μM DM+5.0 μM M (2:2:1 ratio) or GBS.011H as 10.0 μM Y+10.0 μM DM+10.0 μM M (1:1:1 ratio).

21. The method of claim 19, wherein the at least three compounds in a ratio-controlled mixture comprises Yangonin (Y)+Dihydrokavain (DK)+Methysticin (M) as at least one of GBS.012A as 5.0 μM Y+10.0μ M DK+5.0μ M M (1:2:1 ratio), GBS.012B as 5.0 μM Y+10.0μ M DK+10.0 μM M (1:2:2 ratio), GBS.012C as 5.0 μM Y+15.0μ M DK+5.0 μM M (1:3:1 ratio), GBS.012D as 5.0μ M Y+15.0 μM DK+10.0 μM M (1:3:2 ratio), GBS.012E as 10.0 μM Y+10.0 μM DK+5.0 μM M (2:2:1 ratio), GBS.012F as 10.0 μM Y+10.0 μM DK+10.0 μM M (1:1:1 ratio), GBS.012G as 10.0 μM Y+15.0 μM DK+5.0 μM M (2:3:1 ratio) GBS.012H as 10.0 μM Y+15.0 μM+10.0 μM M (2:3:2 ratio).

22. The method of claim 19, wherein the at least three compounds in a ratio-controlled mixture comprises Yangonin (Y)+Cannabidiol (CBD)+Methysticin (M) as at least one of GBS.013B as 5.0 μM Y+0.5 μM CBD+10.0 μM M (10:1:20 ratio), GBS.013D as 5.0 μM Y+1.0 μM CBD+10.0 μM M (5:1:10 ratio), GBS.013E as 10.0 μM Y+0.5 μM CBD+5.0 μM M (20:1:10 ratio), GBS.013F as 10.0 μM Y+0.5 M CBD+10.0 μM M (10:1:20 ratio), GBS.013G as 10.0 μM Y+1.0 μM CBD+5.0 μM M (10:1:5 ratio), or GBS.013H as 10.0 μM Y+1.0 μM CBD+10.0 μM M (10:1:10 ratio).

23. The method of claim 19, wherein the at least three compounds in a ratio-controlled mixture comprises Dihydromethysticin (DM)+Dihydrokavain (DK)+Methysticin (M) as at least one of GBS.014B as 5.0 μM DM+15 μM DK+5.0 M (1:3:1 ratio), GBS.014D as 5.0 M DM+15.0 μM DK+10.0 μM M (1:3:2 ratio), GBS.014E as 10.0 μM DM+10.0 μM DK+5.0 μM M ratio), GBS.014F as 10.0 μM DM+15.0 μM DK+5.0 M (2:3:1 ratio), GBS.014G as 10.0 μM DM+1.0 μM DK+10.0 μM M (1:1:1 ratio), GBS.014H as 10.0 μM DM+15.0 μM DK+10.0 μM M (1:1.5:1 ratio).

24. The method of claim 19, wherein the at least three compounds in a ratio-controlled mixture comprises Dihydromethysticin (DM)+Cannabidiol (CBD)+Methysticin (M) as at least one of GBS.015A as 5.0 μM CBD+0.25 μM CBD+5.0 μM M (20:1:20 ratio), GBS.015B as 5.0 μM DM+0.25μ M CBD+10.0 M (20:1:40 ratio), GBS.015C as 10.0μ M DM+0.25μ M CBD+5.0μ M M (40:1:20 ratio), GBS.015D as 10.0 μM DM+0.25 μM CBD+10.0 μM M (40:1:40 ratio), GBS.015H as 10.0 μM DM+0.5 μM CBD+5.0 M (20:1:10 ratio), GBS.015I as 5.0 μM DM+1.0 μM CBD+5.0 μM M (5:1:10 ratio), GBS.015J as 5.0 μM DM+1.0 μM CBD+10.0 μM M (5:1:5 ratio), GBS.015K as 10.0 μM DM+1.0 μM CBD+5.0 μM M (10:1:5 ratio), or GBS.015L as 10.0 μM DM+1.0 CBD+10.0 μM M (10:1:10 ratio).

25. The method of claim 19, wherein the at least three compounds in a ratio-controlled mixture comprises PEA+B-Caryophyllene (BCP)+Wogonin (WH) as at least one of GBS.ML022B as 0.5 μM PEA+1.0 μM BCP+1.0 μM WH (1:2:2 ratio), GBS.ML022C as 0.5 μM PEA+5.0 μM BCP+0.5 μM WH (1:10:1 ratio), GBS.ML022E as 0.5 μM PEA+5.0 μM BCP+1.0 μM WH (1:10:2 ratio), or GBS.ML022H as 1.0 μM PEA+5.0 μM BCP+1.0 μM WH (1:5:1 ratio).

26. The method of claim 19, wherein the at least three compounds in a ratio-controlled mixture comprises Piperine (P)+Curcumin (CM)+Bacalein (BL) as at least one of GBS.ML023B as 0.5 μM P+0.25 μM CM+1.0 μM BL (1:2:4 ratio), GBS.ML023C as 0.5 μM P+0.5 μM CM+0.5 UM BL (1:1:1 ratio), GBS.ML023E as 1.0 μM P+0.25 μM CM+0.5 μM BL (4:1:2 ratio), or GBS.ML023H as 1.0 μM P+5.0 μM CM+1.0 μM BL (1:5:1 ratio).
